SEQUENCE LISTING (22 sequences)

SEQ ID NO: 1 - DNA, 20 bp, Artificial Sequence
  synthetic nucleotide primer
tctggcggcaccaccatgta

SEQ ID NO: 2 - DNA, 21 bp, Artificial Sequence
  synthetic nucleotide primer
ttgctgatccacatctgctgg

SEQ ID NO: 3 - DNA, 21 bp, Artificial Sequence
  synthetic nucleotide primer
gggctagggaacagatcgacg

SEQ ID NO: 4 - DNA, 20 bp, Artificial Sequence
  synthetic nucleotide primer
gcagcagtcaggtgcggagg

SEQ ID NO: 5 - DNA, 21 bp, Artificial Sequence
  synthetic nucleotide primer
ggcccaataatcagagtggca

SEQ ID NO: 6 - DNA, 23 bp, Artificial Sequence
  synthetic nucleotide primer
ccagtgtcatttccgatcacttt

SEQ ID NO: 7 - DNA, 20 bp, Artificial Sequence
  synthetic nucleotide primer
ggatccaggtatggttccag

SEQ ID NO: 8 - DNA, 20 bp, Artificial Sequence
  synthetic nucleotide primer
ggagcacagtggttgaggat

SEQ ID NO: 9 - DNA, 22 bp, Artificial Sequence
  synthetic nucleotide primer
atgacgtgtgttgaacaagaca

SEQ ID NO: 10 - DNA, 22 bp, Artificial Sequence
  synthetic nucleotide primer
cgatggttgattaaagccaggt

SEQ ID NO: 11 - DNA, 21 bp, Artificial Sequence
  synthetic nucleotide primer
actcggctgagctgagaaatg

SEQ ID NO: 12 - DNA, 19 bp, Artificial Sequence
  synthetic nucleotide primer
gacttgcggtgggtttgtg

SEQ ID NO: 13 - DNA, 23 bp, Artificial Sequence
  synthetic nucleotide primer
atggcttcagacagcatatttga

SEQ ID NO: 14 - DNA, 21 bp, Artificial Sequence
  synthetic nucleotide primer
gtggacgtctctagaaggatt

SEQ ID NO: 15 - DNA, 20 bp, Artificial Sequence
  synthetic nucleotide primer
gccttcagaagagggtgcat

SEQ ID NO: 16 - DNA, 20 bp, Artificial Sequence
  synthetic nucleotide primer
gcactgtgggtacgaaggaa

SEQ ID NO: 17 - DNA, 21 bp, Artificial Sequence
  synthetic nucleotide primer
ccatttcaggtgtcgtgagga

SEQ ID NO: 18 - DNA, 20 bp, Artificial Sequence
  synthetic nucleotide primer
tggcatcgtggacgtctcta

SEQ ID NO: 19 - DNA, 19 bp, Artificial Sequence
  synthetic nucleotide primer
agggcacctccaccatttg

SEQ ID NO: 20 - DNA, 21 bp, Artificial Sequence
  synthetic nucleotide primer
gacactaacacgccgaagtca

SEQ ID NO: 21 - DNA, 20 bp, Artificial Sequence
  synthetic nucleotide primer
ccgatgccagcctccaattt

SEQ ID NO: 22 - DNA, 21 bp, Artificial Sequence
  synthetic nucleotide primer
ctgtgcccattgtactagacg

(19) United States
(12) Patent Application Publication
(10) Pub. No.: US 2016/0130554 A1
(43) Pub. Date: May 12, 2016

(54) Reprogramming Mesenchymal Stromal Cells Into Hematopoietic Cells

(71) Applicant: Wake Forest University Health Sciences, Winston-Salem, NC (US)

(72) Inventors: Maria Graca N.D. Almeida-Porada, Winston-Salem, NC (US); Christopher D. Porada, Winston-Salem, NC (US); Colin E. Bishop, Clemmons, NC (US)

(73) Assignee: Wake Forest University Health Sciences, Winston-Salem, NC (US)

(21) Appl. No.: 14/931,899

(22) Filed: Nov. 4, 2015

Related U.S. Application Data
(60) Provisional application No. 62/077,388, filed on Nov. 10, 2014.

(51) Int. Cl. C12N 5/0789; A61K 35/28

(52) U.S. Cl. CPC C12N 5/0647; A61K 35/28; C12N 2510/00; C12N 2506/13; C12N 2501/60; C12N 2501/603

(57) ABSTRACT

Method for reprogramming of stromal cells into hematopoietic cells are provided. Also provided are mesenchymal stromal cells that can be reprogrammed into hematopoietic cells. In addition, hematopoietic cells reprogrammed from mesenchymal stromal cells are provided. Reprogramming stromal cells into hematopoietic cells is effected through a vector encoding a transcription factor that controls hematopoietic cell differentiation. Methods of treating subjects having diminished hematopoietic cell populations are provided as well.

### CROSS-REFERENCE TO RELATED APPLICATION

This application claims the benefit under 35 U.S.C. §119(e) of U.S. Provisional Patent Application No. 62/077,388, filed Nov. 10, 2014, which is hereby incorporated herein by reference in its entirety.

### STATEMENT AS TO RIGHTS TO INVENTIONS MADE UNDER FEDERALLY SPONSORED RESEARCH AND DEVELOPMENT

This invention was made with government support under grant number R01HL097623 awarded by the National Institute of Health. The government has certain rights in the invention.

### FIELD OF THE INVENTION

The present disclosure relates to reprogramming of stromal cells into hematopoietic cells, and methods and compositions relating thereto.

### BACKGROUND

The hematopoietic system is made up of all adult blood cell types and cells of the myeloid and lymphoid lineages. All of these cells are derived from multipotent hematopoietic stem cells through a succession of precursors with progressively limited potential. Hematopoietic stem cells are tissue-specific stem cells that exhibit remarkable proliferative and self-renewal capacity and are responsible for the life-long maintenance of the hematopoietic system.

Hematopoietic stem cells (HSC) are used clinically. For example, HSC transplants are routinely used to treat patients with cancers and other disorders of the blood and immune systems. These transplants are either autologous (using the subject's own cells) or allogeneic (using donor stem cells, typically from matched donor).

HSC are rare cells that reside in adult bone marrow where hematopoiesis is continuously taking place. They can also be found in umbilical cord blood, and peripheral blood. However, in many instances, these sources do not provide HSC in sufficient numbers for therapeutic purposes or a compatible donor cannot be found. Efforts have also been made to generate hematopoietic stem cells and other hematopoietic cells from induced pluripotent stem cells (iPSCs). However, these efforts have not provided fully mature/functional hematopoietic cells capable of durably engrafting all blood lineages of an adult recipient. In addition, there are clinical concerns regarding the safety/stability of cells generated through an iPSC intermediate derived via forced overexpression of pluripotency transcription factors.

### BRIEF SUMMARY

This disclosure provides compositions of reprogrammed hematopoietic cells, compositions of reprogrammable mesenchymal stromal cells, and methods relating thereto.

In one aspect, provided is an isolated mesenchymal stromal cell comprising a vector, the vector having a nucleic acid sequence encoding a transcription factor that controls hematopoietic cell differentiation. In some instances, the isolated mesenchymal stromal cell is used in the methods described above to generate a hematopoietic cell as described above. In some instances, the mesenchymal stromal cell may express Stro1 but does not express CD34, CD45, and GlyA. In some instances, the mesenchymal stromal cell may be isolated from bone marrow. In some instances, expression of the transcription factor may trigger reprogramming of the mesenchymal stromal cell into a hematopoietic cell.

In some instances, the transcription factor encoded by the vector in the mesenchymal cell may be at least one of RUNX1C or OCT4. In some instances, the vector may include a regulatable promoter operably linked to the nucleic acid sequence encoding the transcription factor. In some instances, the regulatable promoter may be a promoter that permits gene expression to be turned on by addition of an inducer or turned off by addition of a repressor, wherein no or little expression occurs in the absence of the inducer or in the presence of the repressor. In some instances, the vector may have a regulatable promoter operably linked to a nucleic acid sequence that encodes RUNX1C. In some instances, the vector may have a regulatable promoter operably linked to a nucleic acid sequence that encodes OCT4. In some instances, the hematopoietic cell may include a vector having a nucleic acid sequence encoding RUNX1C and a vector having a nucleic acid sequence encoding OCT4. In some instances, at least one of the vectors may have a regulatable promoter operably linked to the nucleic acid sequence encoding RUNX1C or OCT4. In some instances, each of the vectors may have a regulatable promoter operably linked to the nucleic acid sequence encoding RUNX1C or OCT4. In some instances, each of the vectors may have a different regulatable promoter.

In some instances, the mesenchymal stromal cell may reprogram upon expression of the transcription factor into a hematopoietic stem cell (HSC), a hematopoietic progenitor cell (HPC), a myeloid progenitor cell (MPC), a lymphoid progenitor cell (LPC), a lymphocyte, a granulocyte, a macrophage, a erythrocyte, or a platelet. In some instances, the mesenchymal stromal cell may reprogram into a hematopoietic stem cell. In some instances, the mesenchymal stromal cell may reprogram into at least one of a hematopoietic cell that can differentiate into a platelet, a neutrophil, an erythrocyte, or a natural killer cell. In some instances, the mesenchymal stromal cell may reprogram into a hematopoietic cell that can differentiate into at least one of a myeloid lineage, a lymphoid lineage, a megakaryocytic lineage, or an erythroid lineage

In another aspect, provided is a hematopoietic cell containing a vector, the vector comprising a nucleic acid sequence encoding a transcription factor that controls hematopoietic cell differentiation. In some instances, the hematopoietic cell may have been reprogrammed from a mesenchymal stromal cell that expresses Stro1 but does not express CD34, CD45, and GlyA. In some instances, the mesenchymal stromal cell was isolated from bone marrow. In some instances, expression of the transcription factor may trigger the reprogramming of the mesenchymal stromal cell into a hematopoietic cell.

In some instances, the transcription factor encoded by the vector in the hematopoietic cell may be at least one of RUNX1C or OCT4. In some instances, the vector may include a regulatable promoter operably linked to the nucleic acid sequence encoding the transcription factor. In some instances, the regulatable promoter may be a promoter that permits gene expression to be turned on by addition of an inducer or turned off by addition of a repressor, wherein no or little expression occurs in the absence of the inducer or in the presence of the repressor. In some instances, the vector may have a regulatable promoter operably linked to a nucleic acid sequence that encodes RUNX1C. In some instances, the vector may have a regulatable promoter operably linked to a nucleic acid sequence that encodes OCT4. In some instances, the hematopoietic cell may include a vector having a nucleic acid sequence encoding RUNX1C and a vector having a nucleic acid sequence encoding OCT4. In some instances, at least one of the vectors may have a regulatable promoter operably linked to the nucleic acid sequence encoding RUNX1C or OCT4. In some instances, each of the vectors may have a regulatable promoter operably linked to the nucleic acid sequence encoding RUNX1C or OCT4. In some instances, each of the vectors may have a different regulatable promoter.

In some instances, the hematopoietic cell is a hematopoietic stem cell (HSC), a hematopoietic progenitor cell (HPC), a myeloid progenitor cell (MPC), a lymphoid progenitor cell (LPC), a lymphocyte, a granulocyte, a macrophage, an erythrocyte, or a platelet. In some instances, the hematopoietic cell may be a hematopoietic stem cell. In some instances, the hematopoietic cell may be or may differentiate into at least one of a platelet, a neutrophil, an erythrocyte, or a natural killer cell. In some instances, the hematopoietic cell may be of or may differentiate into at least one of a myeloid lineage, a lymphoid lineage, a megakaryocytic lineage, or an erythroid lineage.

In another aspect, provided are methods of generating a hematopoietic cell as described above. The method involves transducing an isolated mesenchymal stromal cell with a vector having a nucleic acid sequence encoding a transcription factor that controls hematopoietic cell differentiation. In some instances, the method may further include culturing the isolated mesenchymal stromal cell in culture medium after transducing the isolated mesenchymal stromal cell with the vector. In some instances, the method may further include harvesting a cell population enriched for hematopoietic cells. In some instances, the mesenchymal stromal cell may be cultured for at least 4 days.

In some instances, the transcription factor encoded by the vector in the mesenchymal cells may be at least one of RUNX1C or OCT4. In some instances, the vector may include a regulatable promoter operably linked to the nucleic acid sequence encoding the transcription factor. In some instances, the regulatable promoter may be a promoter that permits gene expression to be turned on by addition of an inducer or turned off by addition of a repressor, wherein no or little expression occurs in the absence of the inducer or in the presence of the repressor. In some instances, the vector may have a regulatable promoter operably linked to a nucleic acid sequence that encodes RUNX1C. In some instances, the vector may have a regulatable promoter operably linked to a nucleic acid sequence that encodes OCT4. In some instances, the hematopoietic cell may include a vector having a nucleic acid sequence encoding RUNX1C and a vector having a nucleic acid sequence encoding OCT4. In some instances, at least one of the vectors may have a regulatable promoter operably linked to the nucleic acid sequence encoding RUNX1C or OCT4. In some instances, each of the vectors may have a regulatable promoter operably linked to the nucleic acid sequence encoding RUNX1C or OCT4. In some instances, each of the vectors may have a different regulatable promoter.

In some instances, the mesenchymal stromal cell used in the method may be reprogrammable into at least one of a hematopoietic stem cell (HSC), a hematopoietic progenitor cell (HPC), a myeloid progenitor cell (MPC), a lymphoid progenitor cell (LPC), a lymphocyte, a granulocyte, a macrophage, a erythrocyte, or a platelet. In some instances, the mesenchymal stromal cell used in the method may be reprogrammable into a hematopoietic stem cell. In some instances, the mesenchymal stromal cell used in the method may be reprogrammable into at least one of a hematopoietic cell that can differentiate into a platelet, a neutrophil, an erythrocyte, or a natural killer cell. In some instances, the mesenchymal stromal cell used in the method may be reprogrammable into a hematopoietic cell that can differentiate into at least one of a myeloid lineage, a lymphoid lineage, a megakaryocytic lineage, or an erythroid lineage

In some instances, the culture medium may include a compound that alters chromatin modification. In some instances, the compound that alters chromatin modification may be a chromatic-modifying enzyme inhibitor. In some instances, the compound that alters chromatin modification may be a histone methyltransferase inhibitor or a histone deacetylase inhibitor. In some instances, the histone methyltransferase inhibitor may be at least one of Bix-01294, UNC0638, BRDD4770, EPZ004777, AZ505, or PDB4e47. In some instances, the histone deacetylase inhibitor may be at least one of valproic acid, vorinostat, romidepsin, entinostat abexinostat, givinostat, and mocetinostat, butyrate, or a serine protease inhibitor (serpin) family member.

In another aspect, provided are methods of treating a subject having a diminished hematopoietic cell population, the method including identifying a subject having diminished hematopoietic cells, providing hematopoietic cells as described herein, or generated according to the methods described herein, and administering the hematopoietic cells to the subject to repopulate the subject with the hematopoietic cell population. In some instances, the hematopoietic cells may be generated from mesenchymal stromal cells isolated from the bone marrow of the subject.

It will be appreciated from a review of the remainder of this application that further compositions and methods are also part of the disclosure.

### BRIEF DESCRIPTION OF THE DRAWINGS

FIGS. 1A-1E illustrate that MSC marker Stro-1 expressing (Stro-1+) cells contribute to different bone marrow niches during ontogeny according to certain aspects. FIG. 1A provides immunofluorescence analysis images of human fetal bones at 10-12 gestational weeks (gw). Chondrocytes expressing Stro-1 but low/none HSC marker CD34 (CD34^{dim/-}) were detected within the cartilaginous areas (right of dotted line and marked as *, inset at higher magnification), while in the developing bone marrow area (left of dotted line and marked as **) Stro-1+ cells were identified alongside sinusoidal endothelial cells that co-expressed CD34 (diffuse staining in upper left quadrant). The bottom right panel shows a merge of the CD34 and Stro-1 images (overlap of diffuse staining in upper left quadrant). Scale bar represents 50 μm; insets show higher magnification. FIG. 1B provides flow cytometric analysis plots of fetal bone cells for CD45, CD106, CD102, and CD34. The majority of CD34+ cells isolated from fetal bones are vascular CD45− CD34+ cells. FIG. 1C provides immunofluorescence analysis images of cells from fetal bones. Stro-1+ cells co-express HSC marker CD31/PECAM and vascular endothelial marker VE-cadherin (bright punctate staining, solid arrows and inset). Scale bars represent 50 μm. FIG. 1D provides graphs summarizing the percentage of Stro-1+ and VE-Cadherin+ cells during human fetal bone marrow ontogeny at different weeks of gestation. FIG. 1E provides immunofluorescence analysis images of Stro-1+ cells from fetal bones. These cells co-express osteoblast marker osteopontin and endothelial fibroblast marker N-cadherin in areas of forming bone (solid arrows and inset at higher magnification, overlap of staining) Scale bars represent 50 μm.

FIGS. 2A-2E illustrate that the Stro-1+, CD34+ cell subpopulation expresses VEGFR2/KDR/CD309 according to certain aspects. FIG. 2A provides immunofluorescence analysis images indicating that Stro-1+,CD34+,VEGFR2+ cells are present in forming vascular structures (marked as ** below dotted line and insets at higher magnification, areas of brighter staining in insets) at 10-12 gestational weeks (gw). All panels show the same set of cells, with the first and second row showing images using alternating dyes for the different marker, and the third row showing an overlap thereof. FIG. 2B and FIG. 2C provide immunofluorescence analysis images showing that, later on in gestation (17-20 gw), cells co-expressing Stro-1+,CD34+, and VEGFR2+ are identified in specific locations within the vascular structures. In FIG. 2B, all panels show the same area of cells, and co-staining is identified in the area of the structure having an oblong circular shape in the middle top half of the images (arrow in bottom right panel, inset at higher magnification). In FIG. 2C, all panels show the same area of cells, with the top row showing staining for Stro-1, CD34, and VEGFR2 (in green, from left to right), the middle row showing overlap of staining for Stro-1 and VEGFR2, CD34 and Stro-1, and VEGFR2 and CD34 (from left to right), and the bottom row showing the same images as the middle row, but with transmitted light included, to allow visualization of the morphology/structure of the bone tissue. The overlap in staining pattern for Stro-1 and VEGFR2 is indicated by the arrows in middle left panel and insets at higher magnification, which representative of similar overlap in staining for the middle and right panels of the middle row. The arrow head in bottom left panel shows selected area for insert. Scale bars in FIGS. 2A-2C represent 50 μm. FIG. 2D and FIG. 2E provide representative dot plots of flow cytometric evaluation of freshly isolated Stro-1+ cells. Percentages depicted in the plots are after subtraction of respective isotype controls. FIG. 2D provides representative dot plots showing that Stro-1+, CD34+ cells co-express CD31, that VEGFR2/KDR/CD309 is expressed in CD34+/Stro-1+ cells, and that Stro-1+/CD34+ cells are also positive for CD146 and CD49f. In addition, most of the Stro-1+ cells expressed CD90 and CD117. FIG. 2E provides representative dot plots showing that Stro-1+ cells express Apelin Receptor (APLNR/APJ) and are positive for VE-Cadherin/CD144, VEGFR2/KDR/CD309+ and PDGFRα/CD140a.

FIGS. 3A-3I illustrate that fetal Stro-1+ cells acquire a hematopoietic phenotype (CD45+), express CD34+, and are able to generate endothelial cells that efficiently form capillary tubes under appropriate conditions in vitro according to certain aspects. FIG. 3A provides immunofluorescence analysis images identifying CD45+ hematopoietic cells that co-express Stro-1 within the fetal bone marrow. Panels 1 and 4 show Stro-1 and Stro-1 plus CD45 staining at a low magnification, respectively. Panels 2, 3, 5, and 6 are higher magnification images within the insets of Panels 1 and 4. Panels 2 and 3 shows Stro-1 and CD45 staining, respectively, with Panel 5 showing overlap of the staining patterns. Panel 6 shows staining for CD45 and CD117 of the same set of cells. Panel 7 shows the same image as in Panel 4 with transmitted light added to allow visualization of the structure/morphology of the surrounding bone tissue. CD45 staining is bright and encircles the DAPI-stained nuclei of the two cells central to Panels 2, 3, 5, and 6, while Stro-1 staining is more diffuse, with some staining overlapping with the two CD45 stained cells as well as some staining of a third cell position below the two CD45 stained cells. CD117 staining is similar to Stro-1 staining but less extensive. FIG. 3B provides representative flow cytometric analysis plots of freshly isolated adult Stro-1+ cells (SIPs) from bone marrow showing that almost no cells expressed CD34 or CD31, but the cells do express CD117. Percentages listed in the images are after subtraction of respective isotype controls. FIG. 3C provides immunofluorescence analysis images showing that fetal Stro-1+ cells (left column) co-express CD34+ while the adult bone marrow-derived Stro-1+ cells (SIPs) do not (right column). Stro-1 staining is bright in both fetal and adult cells (top row) but there is no CD34 staining of adult cells as shown in the middle right panel. Scale bar represents 50 μm. FIG. 3D provides an agarose gel image of RT-PCR analysis for CD34 mRNA in fetal and in adult Stro-1+ cells. RT-PCR was performed using commercially available primers specific for human CD34. CD34 mRNA transcripts were identified in fetal Stro-1+ cells (lane 1) but not in adult Stro-1+ cells (lane 2). Cord blood-derived HSC were used as positive control (lane 3). A no template control was run simultaneously as a negative control (lane 4). FIG. 3E provides a graph summarizing the tube formation ability of fetal bone marrow (“F BM”) Stro-1+ cells and adult bone marrow (“A BM”) Stro-1+ cells. Cells were grown to fourth passage in media with or without EGM-10 (denoted as “in EGM-10” or “in MSCGM”, respectively) and endothelial cell growth supplements. FIG. 3F provides light microscopy images of fetal and adult Stro-1+ cells grown with or without EGM-10. Fetal cells, but not adult cells, were able to form vasculature without prior induction in EGM-10. Scale bar represents 200 μm. FIG. 3G provides flow cytometry analysis plots of cultured fetal and adult Stro-1 cells at the same passage. The fetal and adult cells express similar levels of the mesenchymal markers CD105, CD146, and CD90, but do not express CD45. FIG. 3H provides microscopy analysis images, using an inverted scope, of fetal and adult Stro-1+ cells grown with or without EGM-2 (denoted as “EGM-2” or “MSCGM” respectively). Both fetal and adult cells differentiate into osteocytes upon induction in EGM-2. FIG. 3I provides flow cytometry analysis plots showing that adult bone marrow Stro-1+ cells express Apelin Receptor (APLNR/APJ) and are mostly negative for VEGFR2/CD309 and PDGFRα.

FIGS. 4A-4E illustrate that Stro-1+ isolated progenitors (SIPs) reprogram in vivo after transplantation into fetal sheep, giving rise to serially transplantable HSC with multilineage potential according to certain aspects. FIG. 4A provides a graph summarizing the percentage of total human hematopoietic (myeloid, lymphoid, and erythroid) engraftment at 2 months post-transplant in fetal sheep transplanted with SIPs. Within each column, each star represents measurement for one animal. FIG. 4B provides a graph summarizing the overall percentage of engraftment in primary and secondary sheep recipients within the different hematopoietic lineages as represented by CD20+(B lymphoid: 0.2-0.8%), CD7+(lymphoid: 1.75-2.25%), CD13+(myeloid: 0.15-0.5%), CD3+(T lymphoid: 0.4-0.9%), GLY A+(erythroid: 1.2-1.4%), CD34+(stem/progenitor cells: 0.2-0.3%). SIPs generated HSC with multilineage differentiation capability in both primary and secondary recipients. FIG. 4C provides a graph summarizing the percentage of human hematopoietic cells in the bone marrow and peripheral blood at 2 months post-transplant in secondary sheep recipients transplanted with HSC generated by in vivo reprogramming in primary sheep recipients. Within each column, each star represents measurement for one animal. FIG. 4D provides flow cytometry analysis plots of CD34+CD45+ cells isolated from bone marrow of primary sheep recipients of SIPs (labeled as “Donor Ch Sh”) and transplanted into NSG mice. Cells were gated based upon FSC, SSC, Ter119−, and 7AAD−. Positive control animals received CD34+CD117+ selected human mobilized peripheral blood stem cells (“Pos. Control”). Negative control animals (“Neg. Control”) were non-transplanted animals. Percentages shown are based on the total number of CD45+ cells in the plot. FIG. 4E provides immunofluorescence analysis images of cultured adult bone marrow-derived Stro-1+ cells (SIP) isolated from two of the secondary recipients that had received SIPs transduced with a lentiviral vector encoding Green Fluorescent Protein (GFP). Bone marrow was collected 2.5 years post-transplant. Human-derived Stro-1 positive, GFP positive cells were detected in culture. The left panel shows bright GFP staining, while the middle panel shows less intense staining. The right panel shows overlap in the staining patterns (brightest areas). Thus, SIPs that did not reprogram to the hematopoietic lineage are also serially transplantable.

FIGS. 5A-5C illustrate in vivo reprogramming of clonally-derived SIP cells of the hematopoietic lineage after transplantation into fetal sheep according to certain aspects. FIG. 5A provides a graph showing the percentage of total human hematopoietic (myeloid/bone marrow and lymphoid/peripheral blood) engraftment at 75 days post-transplantation in sheep transplanted with clonally-derived SIPs. Within each column, each star represents measurement for one animal. FIG. 5B provides a graph summarizing the overall percentage of engraftment within the different hematopoietic lineages as noted above for FIG. 4B. Clonally-derived SIPs generated HSC with multilineage differentiation capability in primary and secondary recipients. FIG. 5C provides a graph summarizing the percentage of human hematopoietic cells in the bone marrow and peripheral blood at 2 months post-transplant in secondary sheep recipients transplanted with HSC generated by in vivo reprogramming in primary sheep recipients. Within each column, each star represents measurement for one animal.

FIGS. 6A and 6B illustrate multilineage hematopoietic engraftment in primary and secondary animals transplanted with adult bone marrow-derived SIPs according to certain aspects. Representative dot plots from flow cytometric analysis of peripheral blood (PB) (FIG. 6A) and bone marrow (BM) (FIG. 6B) of primary and secondary recipients as well as a nontransplanted control are shown. Transplanted animals were considered positive for a specific lineage if the percentage of positive events were higher than the nontransplanted control animal. The percentage of cells indicated in the quadrants represents the percentage of cells staining with the respective antibody after subtraction of the relevant isotype control.

FIG. 7 illustrates reprogramming of stromal cells using OCT4 according to one aspect. SIPs were transduced with a lentivector encoding OCT4 and placed in induction media to promote reprogramming, as detailed in materials and methods. An image of untransduced SIPs appears in the left panel, while the image on the right shows a representative colony that formed when SIPs were induced to reprogram via forced expression of OCT4.

FIGS. 8A-8C illustrate reprogramming of stromal cells using OCT4 to induce expression of early hematopoietic markers according to one aspect. Colonies that formed as a result of Oct4-induced reprogramming of SIPs were stained with fluorescently-labeled antibodies to the hematopoietic markers CD45 and CD34. FIG. 8A shows a microscopy image in phase contrast of a representative OCT4-expression driven reprogrammed colony. FIG. 8B shows the same colony stained with FITC-labeled anti-CD45 (all staining in image is CD45). FIG. 8C shows the same colony stained with PE-labeled anti-CD34 (all staining in image is CD34). FIG. 8D shows a composite image of FIG. 8C and FIG. 8D, demonstrating co-expression of CD45 and CD34 in the cells comprising the colony.

FIG. 9 provides a heatmap illustrating reprogramming of stromal cells as compared to fibroblast cells using OCT4 according to certain aspects. Human foreskin fibroblasts (HFF) and SIPs were transduced with a lentivector encoding OCT4. RNA was isolated from these two populations and from untransduced HFF and SIPs as baseline controls. mRNA was isolated from the samples and converted into cDNA. Microarray analysis of the cDNA samples was used to generate the heatmap, which demonstrates the marked difference between the transcriptome of HFF and SIPs, both prior to and following forced expression of OCT4. As is convention in gene array heatmaps, genes that are upregulated appear red (medium dark areas), while those that are downregulated appear blue (darkest areas). Genes whose expression does not differ between the groups being compared appear as a grayish color. As indicated by the color scale bar across the bottom of the figure, the intensity of the color indicates the degree to which the expression of the gene in question differs, with bright red (medium dark areas) indicating a ≧1.5-fold upregulation, and bright blue (darkest areas) indicating a ≧1.5-fold down regulation.

FIG. 10 provides representative FACS plots showing reprogramming of stromal cells by OCT4 and RUNX1C to express early hematopoietic markers according to certain aspects. SIPs were transduced with a lentivector encoding OCT4+RUNX1C and analyzed at various time points by flow cytometry for induction of expression of CD41, the earliest marker of commitment to the hematopoietic lineage. Forced expression of OCT4+RUNX1C led to rapid upregulation of CD41. This expression peaked at day 5-6 post-transduction, at which time nearly 20% of SIPs had been reprogrammed sufficiently to express this early hematopoietic marker (n=5).

FIG. 11 provides a graph summarizing data of reprogramming of stromal cells by OCT4 and RUNX1C to have co-expression/upregulation of CD41 and other hematopoietic-specific genes according to certain aspects. At 5 days after transduction with a lentivector encoding OCT4+RUNX1C (at which time CD41 expression is maximal), RNA was isolated from SIPs and analyzed by qRT-PCR for expression of genes associated with early hematopoietic stem/progenitor cells. Maximal expression of CD41 coincided with robust induction of CD34 (65-fold), CD45 (295-fold), and KDR (179-fold) mRNA (n=6).

FIG. 12 provides a graph summarizing data of reprogramming of stromal cells by OCT4 and RUNX1C to have induced expression of multiple hematopoiesis-specific transcription factors according to certain aspects. At 5 days after transduction with a lentivector encoding OCT4+RUNX1C, RNA was isolated from SIPs and analyzed by qRT-PCR for expression of transcription factors known to be involved in hematopoietic fate commitment/early hematopoiesis. As can be seen here, maximal expression of CD41 coincided with robust induction of multiple hematopoiesis-specific transcription factors (n=6).

FIG. 13 provides a graph summarizing data of reprogramming of stromal cells by RUNX1C, valproic acid (VPA), and Bix-01294 according to certain aspects. SIPs were transduced with a lentivector encoding RUNX1C alone and exposed to VPA and Bix-01294. Replacing OCT4 with these small molecules that alter chromatin accessibility further enhanced the reprogramming of SIPs to the hematopoietic lineage, with 10% and 35% of SIPs successfully reprogramming to express CD34 (gray bars) and CD41 (black bars), respectively, within only 4 to 5 days.

FIG. 14 provides a graph summarizing RUNX1C expression patterns in reprogrammed stromal cells according to certain aspects. RNA isolated from SIPs on days 6-12 following transduction with a lentivector encoding RUNX1C, and qRT-PCR was performed to analyze the levels of endogenous RUNX1C (black checkerboard), exogenous (lentivector-driven; black and white vertical stripes) RUNX1C, and total RUNX1 (A+B+C; hatched diagonal lines). The bar graph depicts the relative levels of exogenous vs. endogenous RUNX1C expressed as a percentage of total RUNX1 (A+B+C), which has been set to 100% to enable comparison.

### DETAILED DESCRIPTION

Certain embodiments and features of the present disclosure relate to mesenchymal stromal cells that can be reprogrammed into hematopoietic cells. Also, certain embodiments and features relate to hematopoietic cells that have been reprogrammed from mesenchymal stromal cells. In addition, certain embodiments and features relate to methods of generating hematopoietic cells from mesenchymal stromal cells by reprogramming the mesenchymal stromal cells into hematopoietic cells. Also disclosed are methods of treating a subject having a diminished hematopoietic cell population, such as a hematopoietic defect, deficiency, or disease, involving administering hematopoietic cells that have been reprogrammed from mesenchymal stromal cells to the subject.

A population of mesenchymal stromal cells (MSCs) has been discovered that can be reprogrammed to generate hematopoietic stem cells (HSCs). This MSC population was assessed in an in vivo environment, and it was determined that this cell population has the ability to reprogram into HSCs. As described herein, processes have been developed for in vitro reprogramming of these MSCs into HSCs. MSCs may be used as a source of somatic cells that can be efficiently, safely, and completely reprogrammed in vitro into multipotent, long-term repopulating HSCs and other hematopoietic cells. Reprogrammed hematopoietic cells may be used in clinical applications to treat patients with a broad variety of blood diseases.

In one aspect, Stro-1 is a cell surface protein expressed by these mesenchymal stem/stromal cells. In another aspect, these cells do not express hematopoietic markers such as CD34 (hematopoietic progenitor cell antigen), CD45 (an antigen expressed on all leukocytes), and GlyA (an antigen expressed on all red blood cells). In one aspect, this MSC population is not committed to the hematopoietic lineage. This MSC population can be isolated from bone marrow, as well as from tissues such as brain, adipose tissue, liver, lung, gut, and dental pulp, and also in umbilical cord tissue using methods such as cell sorting and/or flow cytometry. For example, cell sorting and flow cytometry may be used to isolate the MSC based on expression of Stro-1. This cell population will be referred to generally throughout this disclosure as Stro-1+ Isolated Stromal Progenitors or SIPs. However, “mesenchymal stromal cell”, “mesenchymal cell”, “stromal cell”, “Stro-1+ Isolated Stromal Progenitors” and “SIPs” are generally referred to interchangeably throughout this disclosure.

In one aspect, reprogramming of mesenchymal stromal cells such as SIPs can be performed by in vitro (exogenous) expression of a transcription factor that controls hematopoietic cell differentiation encoded on a vector introduced into the MSC. For example, the transcription factor that is expressed exogenously may be at least one of RUNCX1 or OCT4. In one aspect, OCT4 is a transcription factor associated with generalized reprogramming and/or pluripotency. In another aspect, RUNX1C is a transcription factor associated with hematopoietic differentiation. In some instances, the MSC are also cultured in vitro in the presence of a compound that alters chromatin modification such as a chromatic-modifying enzyme inhibitor. In some instances, culturing the MSC that are exogenously expressing a transcription factor that controls hematopoietic cell differentiation with a compound that alters chromatin modification may facilitate reprogramming by altering epigenetic imprinting of the MSC. Such compounds may be histone methyltransferase inhibitors and/or histone deacetylase inhibitors.

As discussed herein, a number of markers (biomarkers/biological markers) are described that may be used to identify and characterize cell populations, including the Stro-1 MSC population and the cell compositions provided herein. As referenced herein, and as known in the art, markers include genes or gene products, such as mRNA and protein, which can be assessed, detected and/or measured when expressed by a cell. For example, proteins expressed on the cell surface may be detected and utilized as markers by a variety of means including, for example, cell sorting, flow cytometry, and immunohistochemistry as well as by other techniques. In another example, mRNA expression of genes may be detected and utilized as markers by methods including, for example, polymerase chain reaction (PCR). An exemplary biomarker used to identify the MSC population described herein is Stro-1, which is a protein expressed on the surface of the cells. Also described herein are exemplary markers CD31/PECAM, a marker of endothelial cells, CD45, a marker of leukocytes, GlyA, a marker of red blood cells, CD34 a marker of hematopoietic stem/progenitor cells (HSC), and VEGFR2/KDR/CD309, a marker of a subset of HSC, endothelial progenitor cells (EPCs), and their common progenitor, the hemangioblast. In some instances, expression of VE-cadherin, CD31/PECAM-1, CD34, and VEGFR2 may be associated with hematopoietic potential. Exemplary markers representative of different hematopoietic lineages include CD20 (B lymphoid), CD7 (lymphoid), CD13 (myeloid), CD3 (T lymphoid), GlyA (erythroid), CD34 (hematopoietic stem/progenitor cells). Also described herein are exemplary hemangioblast markers such as ANGPT1, KIT, MEIS1 and 2, NPR3, HEX, DLX5, CRHBP, GATA2, HLF, and PROM-1. Also described herein are exemplary markers associated with commitment to the hematopoietic lineage such as SCL, RUNX1B, HHEX, KLF2, NFE2, RUNX1C, LYL-1, LMO-2, GATA-2, PU.1, C-MYC ERG, FLI-1, GFIB, MLL, HOXB4, and CDX4. Exemplary markers of mesodermal precursors/mesenchymal progenitors are Apelin Receptor (APLNR/APJ) and PDGFRα/CD140a. Other markers described herein include CD146, a marker of mesenchymal stem cells from various sources, as well as endothelial progenitor cells and pericytes, CD49f/integrin α6, a marker of hematopoietic stem cells, CD90, a marker of hematopoietic and mesenchymal stem cells, and CD117 (c-kit), a marker of hemogenic endothelium, hemangioblasts, and hematopoietic stem cells, as well as stem cell populations within other tissues of the body. An exemplary vascular endothelial marker is VE-cadherin/CD 144. As used herein, expression of a biomarker by a cell may be indicated by a “+” symbol following the marker name, while lack of expression of a biomarker by a cell may be indicated by a “−” symbol following the marker name.

A. Compositions and Methods of Making

Disclosed are isolated mesenchymal stromal cells that are reprogrammable in vivo and in vitro into hematopoietic cells. In one aspect, the isolated mesenchymal stromal cells may contain a vector that includes a nucleic acid sequence encoding a transcription factor that controls hematopoietic cell differentiation. In another aspect, the vector permits in vitro reprogramming of the isolated mesenchymal cells into hematopoietic cells. Also disclosed are reprogrammed hematopoietic cells. In one aspect, the reprogrammed hematopoietic cells contain a vector that includes a nucleic acid sequence encoding a transcription factor that controls hematopoietic cell differentiation. Provided herein are aspects and features of the isolated mesenchymal stromal cells and their ability to reprogram in vivo and in vitro into hematopoietic cells, and hematopoietic cells that have been reprogrammed in vitro from such isolated mesenchymal stromal cells. Also provided are methods of generating hematopoietic cells by in vitro reprogramming isolated mesenchymal cells.

1. Isolated Mesenchymal Stromal Cells and In Vivo Reprogramming

Characterization of fetal and adult Stro-1 expressing mesenchymal stromal cells is described in this section. In some instances, the fetal and adult Stro-1+ cells may be assessed after collection. In other instances, the fetal and adult Stro-1+ cells may cultured in vitro and then assessed. In some instances, fetal Stro-1+ cells and adult Stro-1+ cells may both express particular biomarkers. In other instances, fetal Stro-1 cells may express biomarkers that adult Stro-1+ cells do not express. In other instances, adult Stro-1 cells may express biomarkers that fetal Stro-1+ cells do not express.

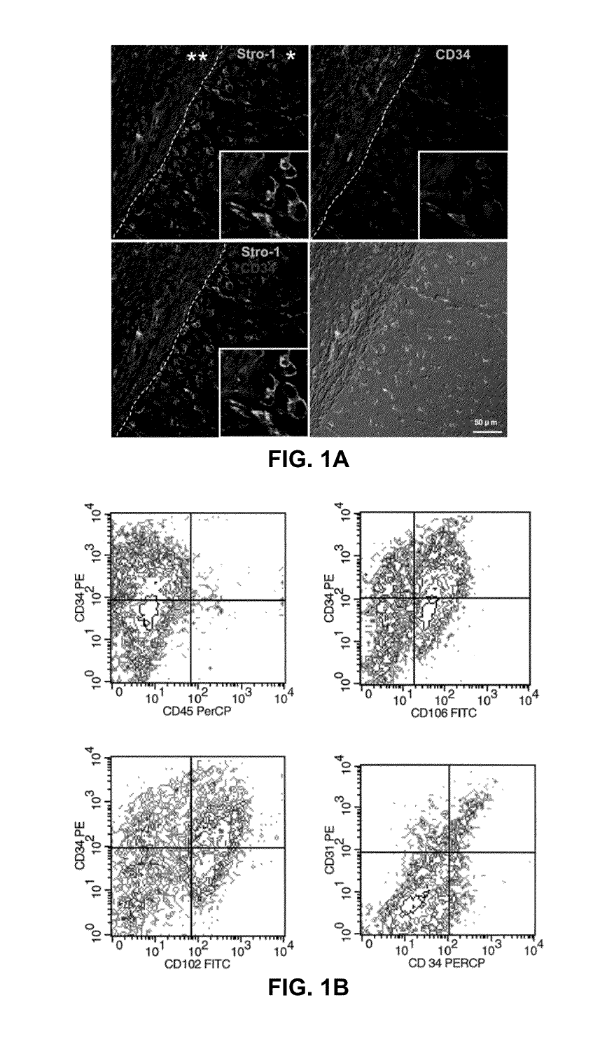

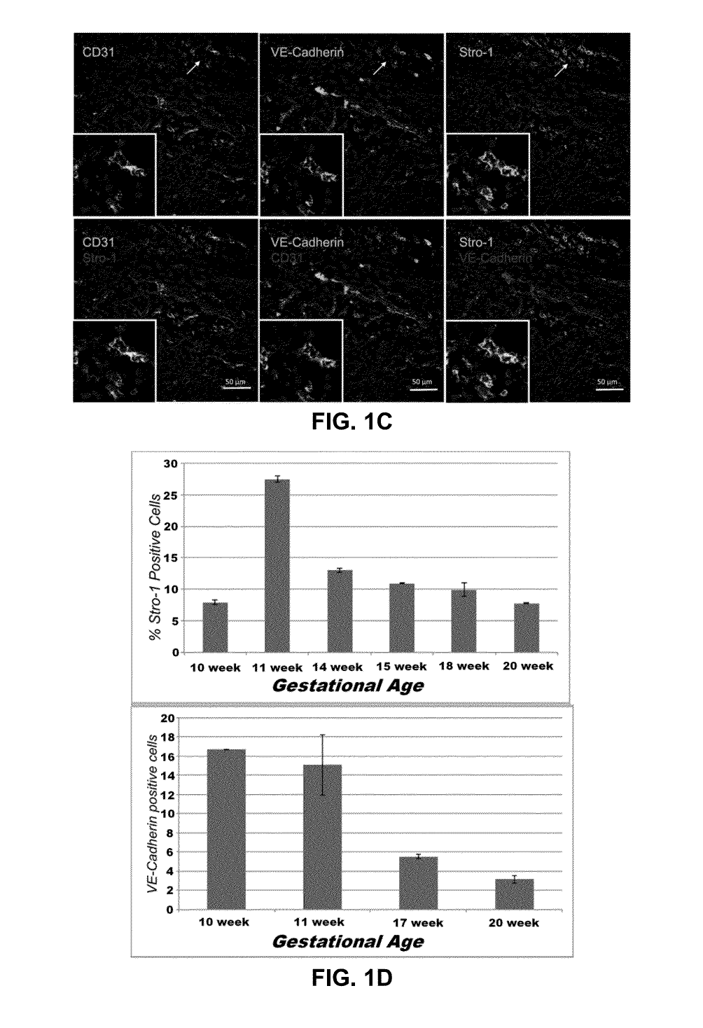

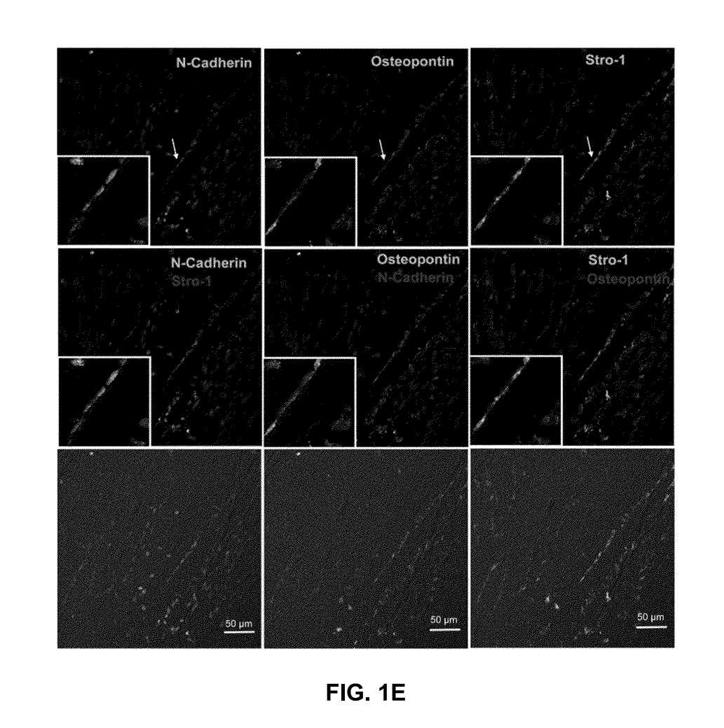

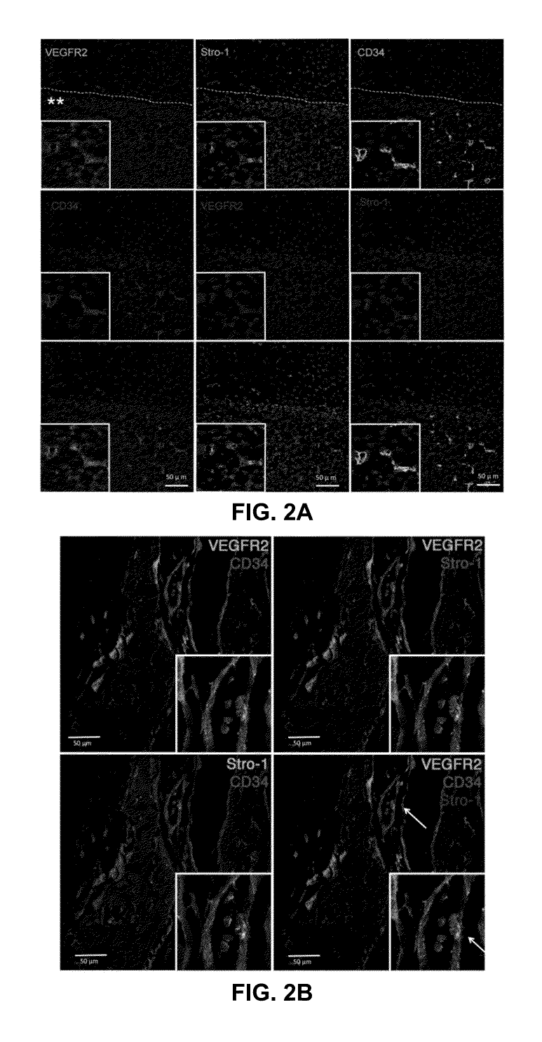

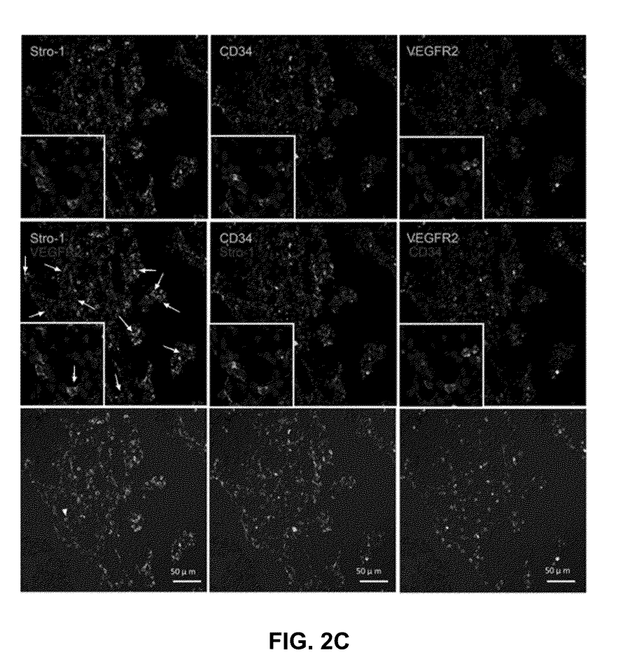

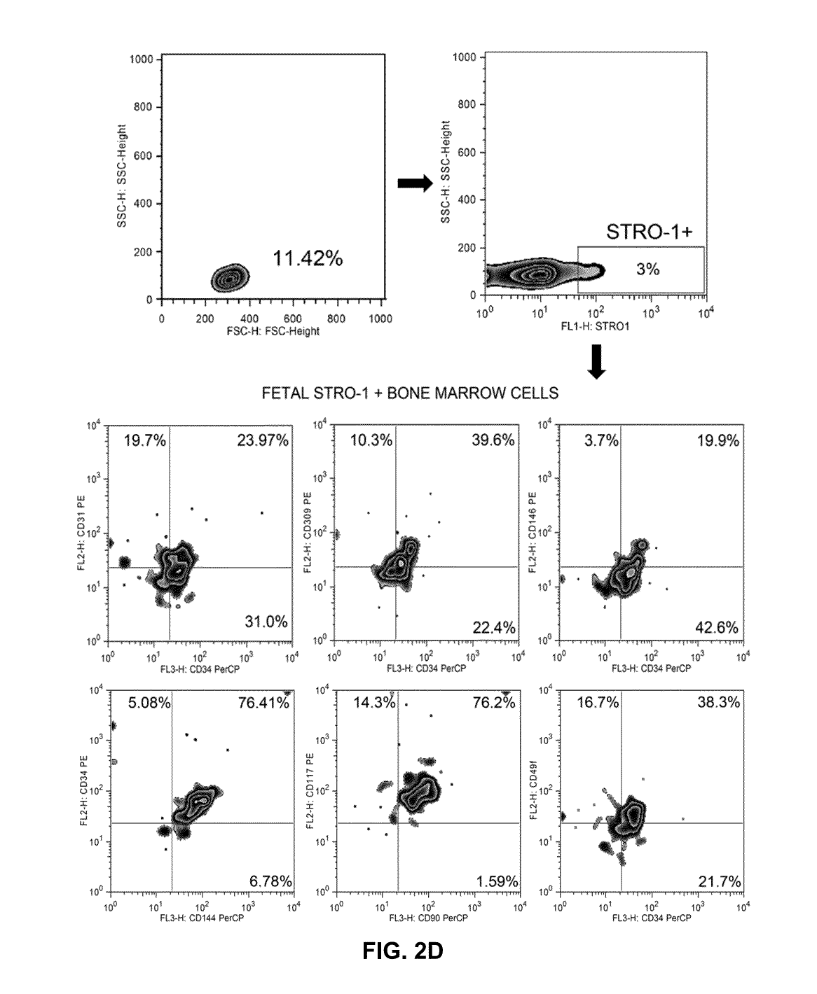

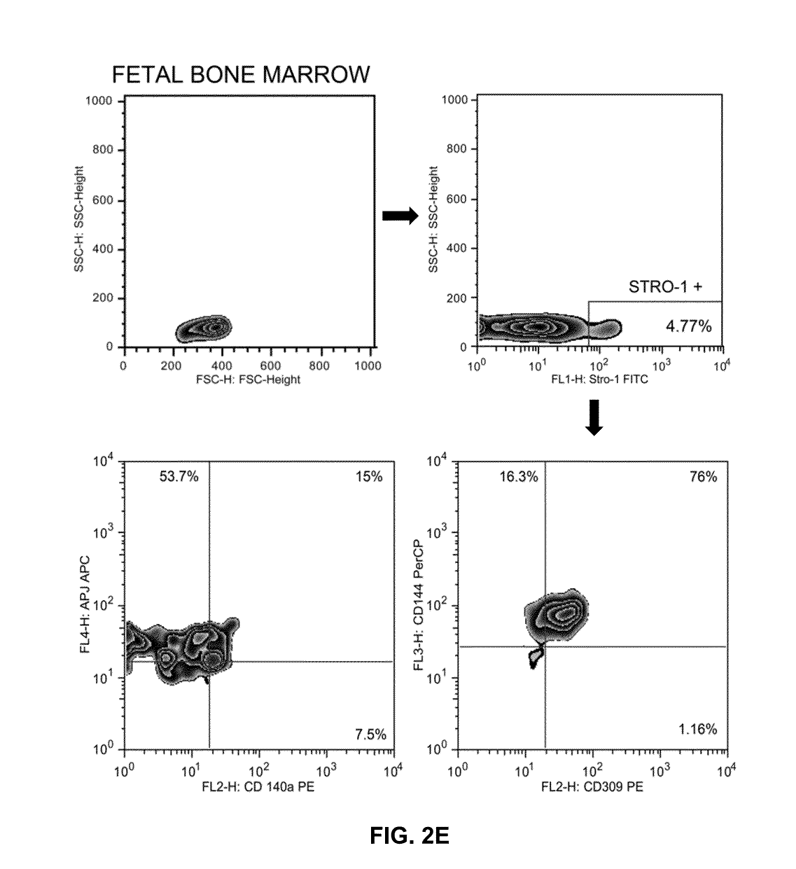

In some instances, fetal Stro-1+ cells may contribute to different bone marrow niches during fetal development. In certain cases, Stro-1+ cells may be identified in chondrogenic-forming areas earlier in development (such as around 10-11 gw) and as osteoblastic cells expressing osteopontin and N-cadherin slightly later in development (such as around 11-12 gw). In some instances, fetal cells co-expressing Stro-1, CD34, and VEGFR2 may be present in vascular structures as shown, for example, in FIG. 1A. In some aspects, fetal Stro-1+ cells from the mesenchymal niche may differentiate/contribute to vasculature, cartilage, and bone during fetal development. In some instances, Stro-1+ cells can also be identified in fetal bones, at various maturation states, by expression of biomarkers including, but not limited to, Stro-1+, CD34, VEGFR2, CD49f, CD146, CD90, CD117, PDGFRα, and VE-Cadherin. In some instances, Stro-1+ cells may co-express CD31/PECAM and VE-cadherin as shown, for example, in FIG. 1B. In some instances, fetal Stro-1+ cells express osteopontin and N-cadherin in areas of forming bone as shown, for example, in FIG. 1D. In certain instances, fetal cells expression Stro-1, CD34, and VEGFR2 may be present later in development in vasculature structures as shown, for example, in FIG. 2E. In some instances, hematopoietic cells may develop from the sites where these Stro-1+, CD34+, VEGFR2+ cells are present.

In various aspects, fetal Stro-1+ cells may express particular biomarkers. In some instances, fetal Stro-1+ cells may have different properties and/or express different markers than adult Stro-1+ cells. For example, fetal Stro-1+ cells may express marker CD34 while adult Stro-1+ cells (such as those isolated from bone marrow) do not express CD34 as shown in FIGS. 2A and 2B. For example, the Stro-1+ cell population within fetal bone marrow express APJ/APLNR, a marker of mesodermal precursors. In another example, fetal Stro-1+ cells express APJ and PDGFRα+, markers of hemogenic endothelium, as shown in FIG. 2E. In certain examples, Stro-1+,VEGFR2+,CD34+ cells may be present during fetal bone marrow development and may contribute to the vasculogenic process as shown in FIG. 2A-2C. In some instances, the Stro-1+, CD34+ cells may also express at least one of VEGFR2, CD146, or CD49f as shown in FIG. 2D. In some instances, the Stro-1+ cells may also express at least one of CD90 or CD117 as shown in FIG. 2D. Confocal microscopy may be used to identify this cell population. In some examples, the Stro-1+ cells may express VE-Cadherin/CD140a and/or VEGFR2/CD309 as shown in FIG. 2E.

In some instances, fetal Stro-1+ cells cultured in vitro may continue to express hematopoietic-specific marker CD34 but may not express hematopoietic-specific marker VEGFR2 (KDR/CD309). For example, some Stro-1+, CDR34+ cells express VEGFR2 as shown in FIG. 2D. In some instances, these cells may not be able to produce hematopoietic colonies in methylcellulose culture. Without being held to any specific theory, it is possible that the compromised hemogenic ability of fetal Stro-1+ cells in vitro could be due to culture conditions which are known to lead to altered epigenetics and differentiation potential (see for example Shoshani et al. 2014). In another aspect, fetal Stro-1+, CD34+ cells may maintain efficient angiogenic capacity as demonstrated by their ability to efficiently form vascular tubes. For example, fetal Stro-1+, CD34+ cells may maintain angiogenic capacity even after being cultured in media not designed to support the growth of endothelial cells.

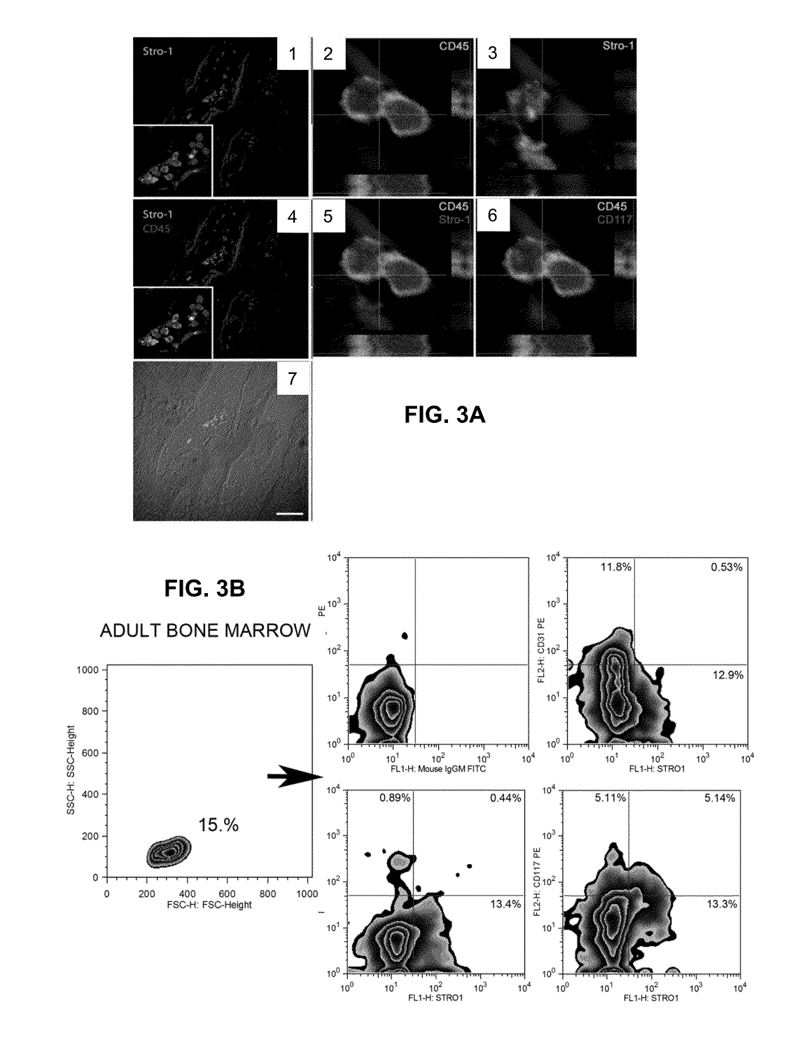

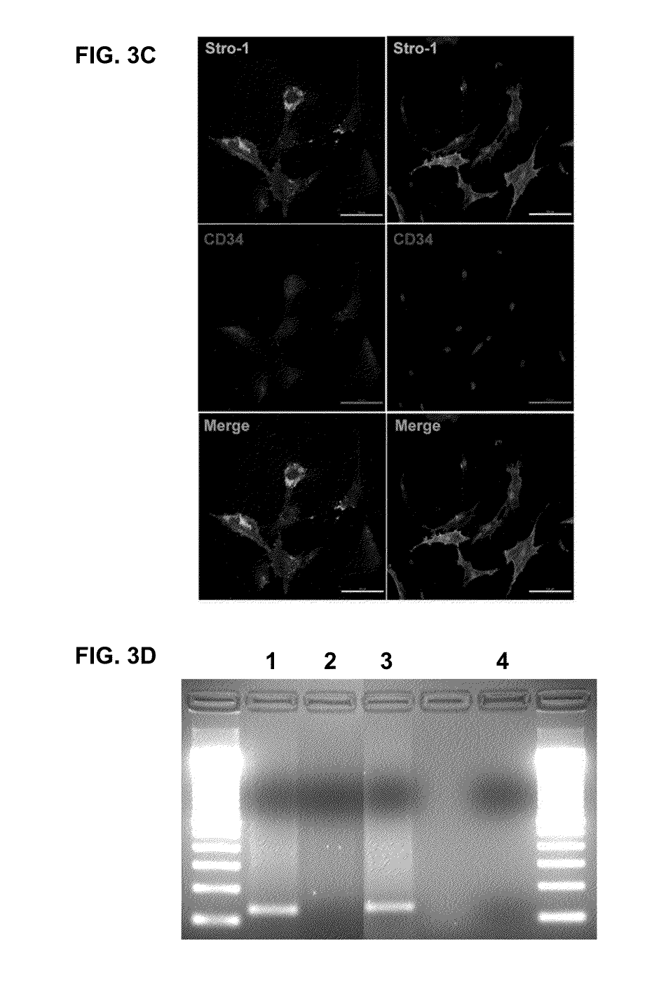

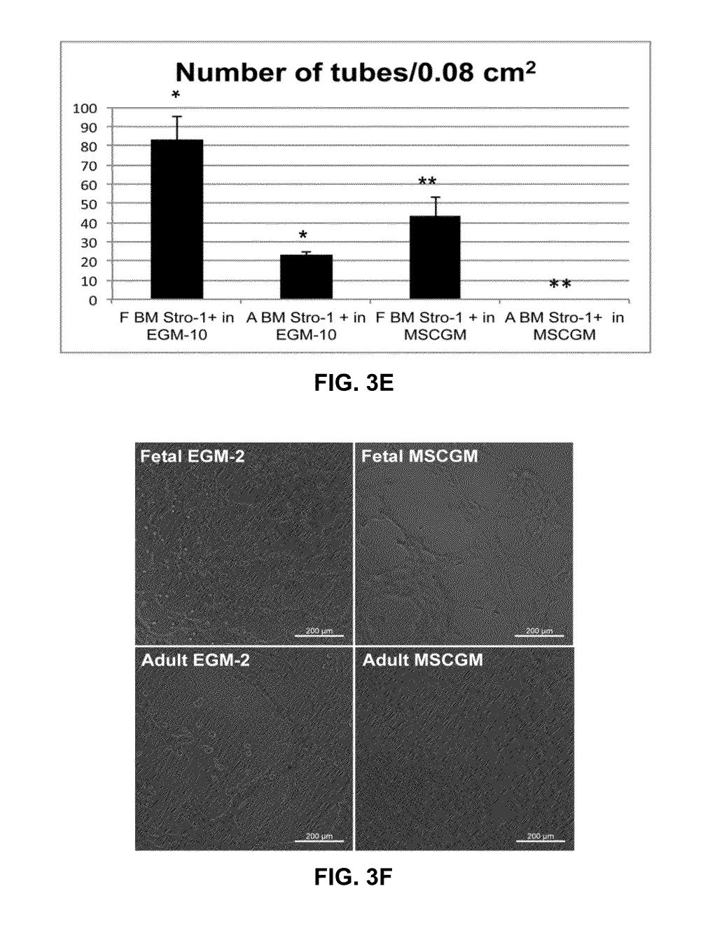

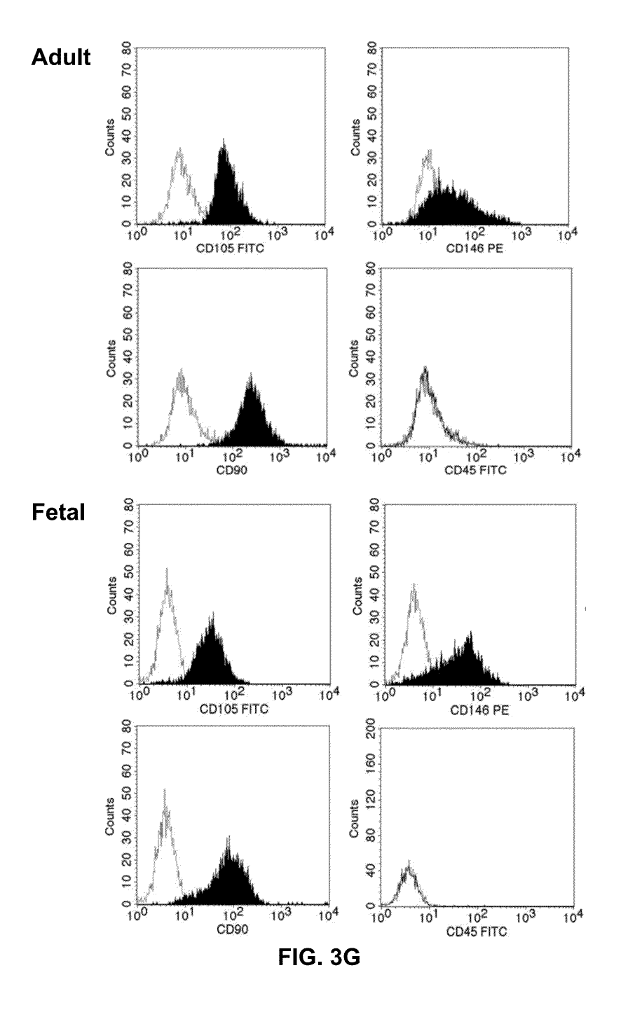

In some instances, adult SIPs are phenotypically and functionally different from fetal Stro-1+ cells. In certain instances, fetal Stro-1+ cells begin to acquire a hematopoietic phenotype as characterized by expression of CD45 at about 14 gw, for example, as shown in FIG. 3A. In another aspect, adult Stro1+ isolated stromal progenitors (SIPs) may not express CD34, CD45, or GlyA as shown, for example, in FIGS. 3B and 3C. In one example, mRNA expression of CD45 is present in fetal Stro-1+ cells but not in cultured adult Stro-1+ cells as shown, for example, in FIG. 3D. These cells may be isolated from bone marrow, adipose tissue, brain, liver, lung, gut, dental pulp, and umbilical cord tissue. In some instances, SIPs may be cultured with a cocktail of growth factors to induce formation of vascular structures as shown, for example, in FIGS. 3E and 3F. In one example, adult SIPs may form vascular structures with less efficiency than their fetal counterparts. In one example, fetal Stro-1+ cells may be able to form vasculature without prior induction in endothelial-supporting medium, while adult Stro-1+ cells may require induction in endothelial-supporting medium to do so as shown in FIG. 3E-3F. In some instances, freshly isolated adult SIPs from bone marrow may express APJ but may not express PDGFRα or other markers of hematovascular mesodermal precursors as shown, for example, in FIG. 3G. In some instances, adult Stro-1+ cells may be induced to differentiate into osteocytes using the appropriate media as shown, for example, in FIG. 3H. In some instances, both fetal and adult Stro-1+ cells may differentiate into osteocytes upon induction in appropriate medium as shown in FIG. 3H. In some instances, freshly isolated adult Stro-1+ cells, but not fetal Stro-1+ cells, may express APJ but may not express hematovascular precursor markers as shown, for example, FIG. 3I. However, in organs besides the bone marrow, Stro-1 identifies antigens present in perivascular and endothelial cells (Ning et al., 2011; Soland et al., 2014). Without being held to any particular theory, it is possible that alterations in the transcriptional profile of adult SIPs occur as they adopt their known mature mesenchymal phenotype. For example, the environment may have a direct role on the post-development human epigenome. The local tissue environment may directly modulate DNA methylation patterns in normal differentiated cells in vivo. Also, in some instances, cells may lose their tissue-specific epigenetic landscape in a time-dependent manner following exposure to another environment.

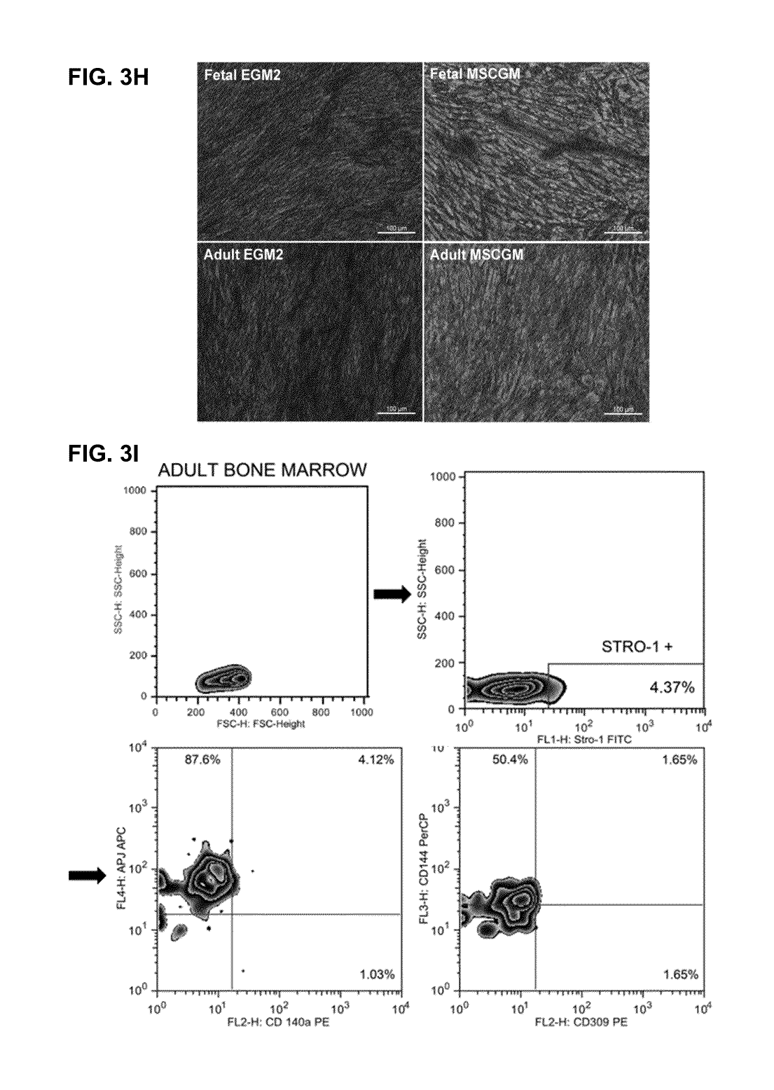

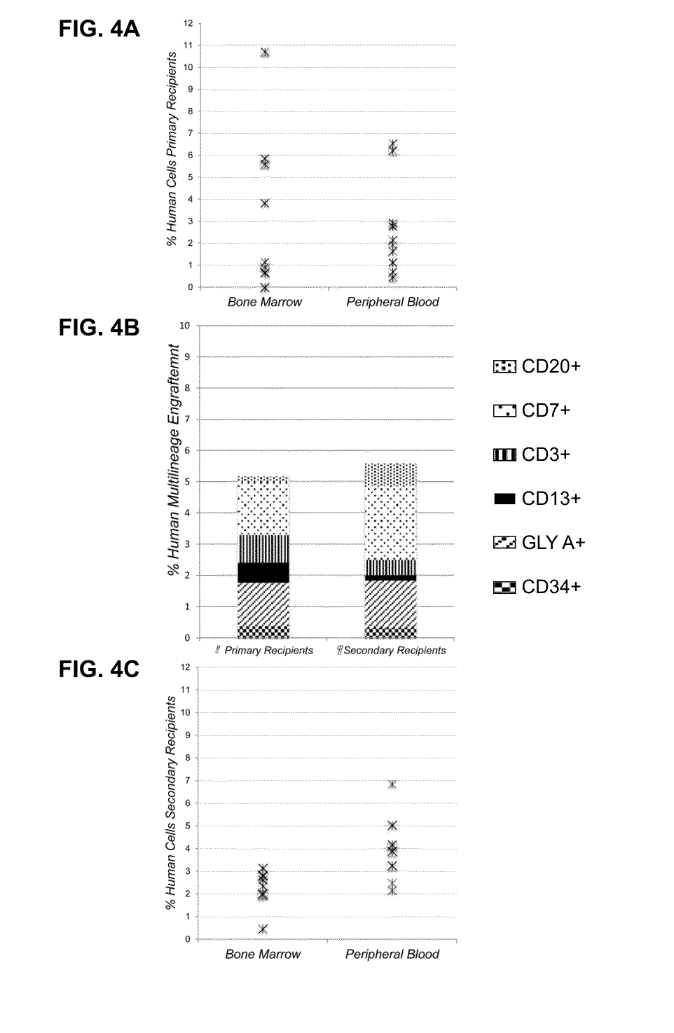

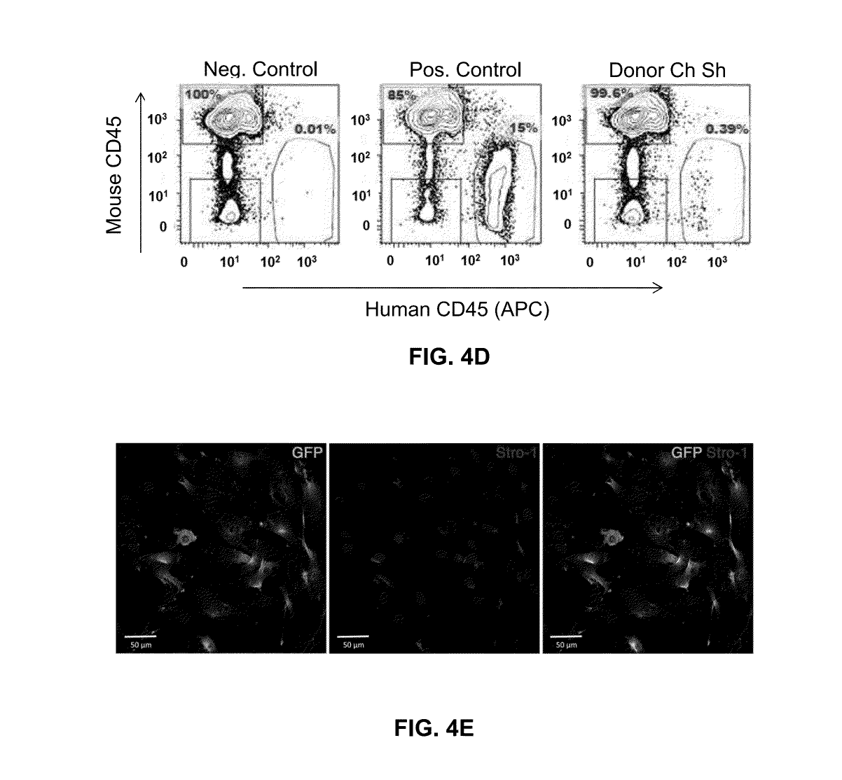

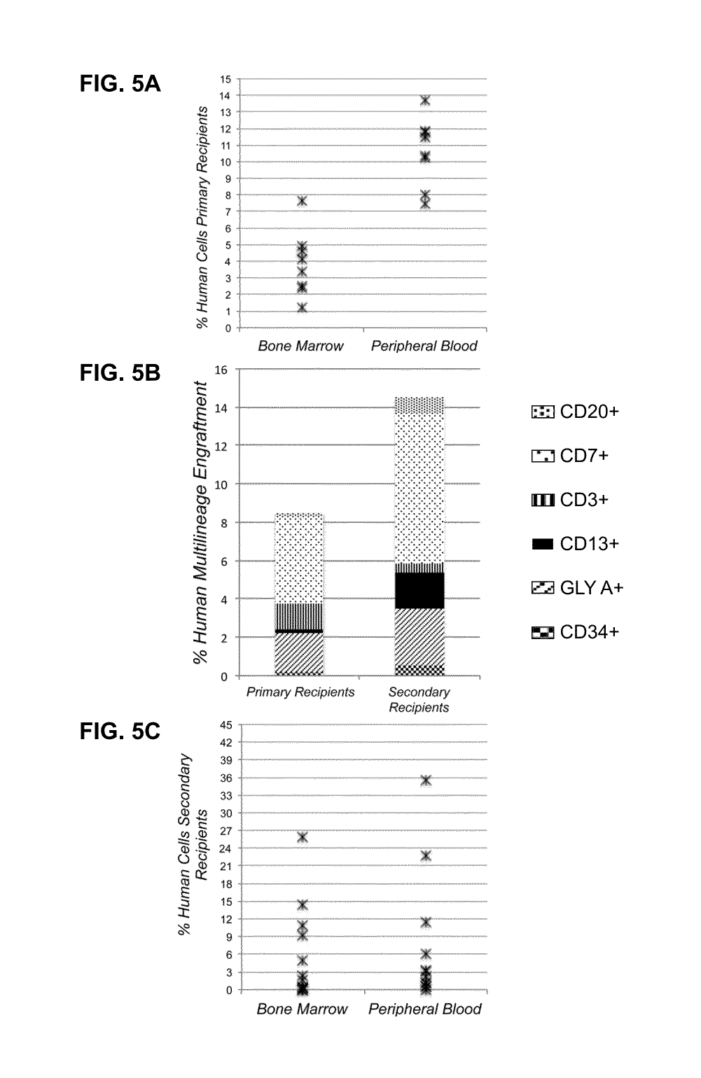

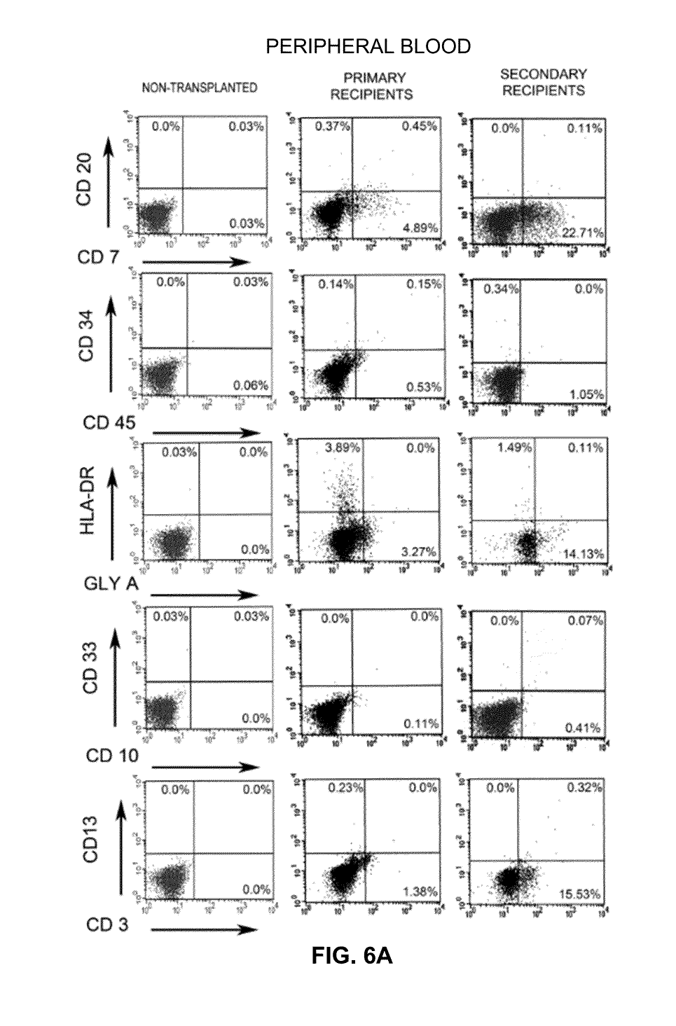

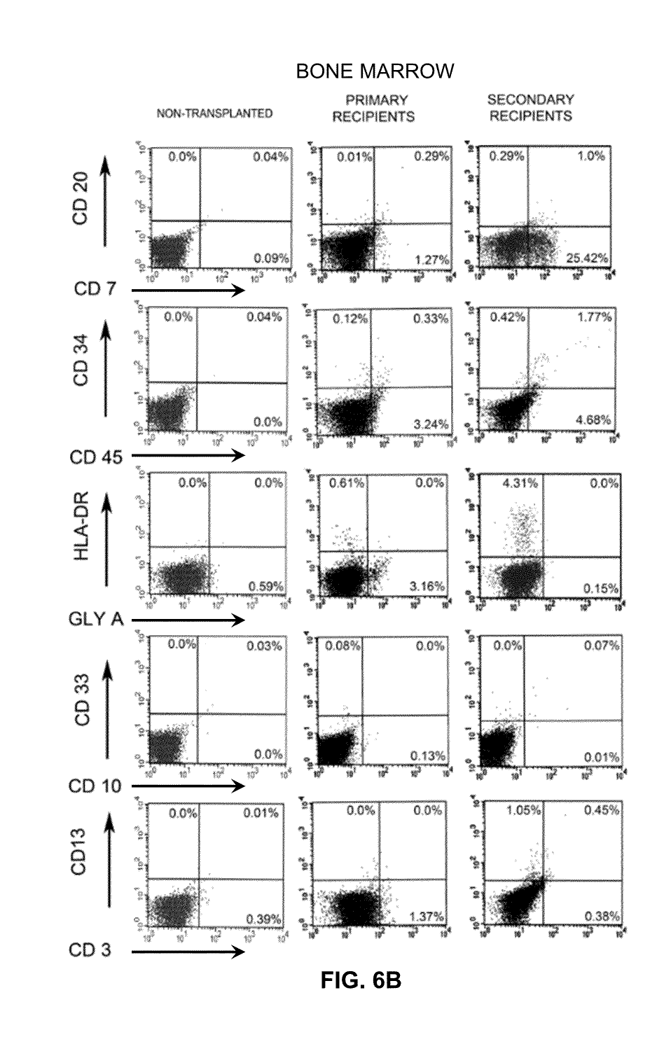

In another example, adult Stro-1+ mesenchymal stromal cells (SIPs) can reprogram when placed in vivo in a fetal environment to a more primitive phenotype. For example, upon reprogramming, SIPs may generate hematopoietic stem cells (HSC) capable of robust, serial, multilineage reconstitution. In one example, exposure in vivo to a bone marrow niche microenvironment whose development parallels that of human may cause adult human SIPs to reprogram to an earlier fetal precursor state and give rise to hematopoietic cells as shown, for example, in FIG. 4A, which shows experiments with clonally derived SIPs. In another aspect, the reprogrammed hematopoietic cells may be capable of robust multilineage reconstitution of the hematopoietic systems of primary and secondary recipients as shown, for example, in FIGS. 4B and 4C, which show experiments with clonally derived SIPs. In some instances, engrafted Stro-1+ cells may show characteristic expression patterns for CD20, CD34, HLA-DR, CD33, CD13, and CD3 in primary and second sheep recipients as compared to non-transplanted control sheep as shown, for example, in FIGS. 6A and 6B. In certain instances, non-clonally derived SIPs may also serially engraft, reprogram into hematopoietic cells, and differentiate similarly into both myeloid and lymphoid cells as show, for example, in FIG. 5A-5C. In some instances, HSC generated from transplanted SIPs may have behavior identical to that of native HSC. For example, human HSC generated from SIPs injected into primary sheep may be secondarily transplanted into NSG mice and may be able to engraft and generate CD45+ cells as shown, for example, in FIG. 4D. The characteristics of the Stro-1+ cells may be in contrast to bone marrow-derived stromal cells that were not isolated based upon Stro1-positivity that may not be able to generate detectable levels of hematopoietic cells when transplanted into this same fetal model (see, for example, Almeida-Porada et al. 2000 and 2000). In some instances, SIPs that did not reprogram to the hematopoietic lineage upon introduction into a fetal sheep microenvironment may engraft long-term in a secondary recipient as shown, for example, in FIG. 4E.

In some instances, the ability of clonally-derived SIPs to generate HSC that were serially transplantable establishes that SIPs may be amenable to microenvironmental induction, at the clonal level, and may have the ability to reprogram in vivo to a hematopoietic phenotype. In some instances, serially transplanted recipients with the highest levels of hematopoietic engraftment may exhibit expansion of the erythroid or lymphoid compartment based on the engrafted SIPs.

2. In Vitro Reprogramming Methods

In one aspect, a method of generating reprogrammed hematopoietic cells may include transducing an isolated mesenchymal stromal cell with a vector comprising a nucleic acid sequence encoding a transcription factor that controls hematopoietic cell differentiation. In one aspect, upon transduction of the isolated mesenchymal cell with the vector and induction of expression of the transcription factor, the transcription factor triggers the reprogramming of the mesenchymal stromal cell into a hematopoietic cell.

In various aspects, the isolated mesenchymal stromal cell may have the characteristics and properties as set forth above in Section A.1. Mesenchymal stromal cells may be isolated from various sources. Exemplary sources include, for example, bone marrow, adipose tissue, brain, liver, lung, intestine, dental pulp, and umbilical cord tissue. In one example, the mesenchymal stromal cells may be isolated from bone marrow. The mesenchymal stromal cells may be isolated from fetal or adult sources. In one example, the mesenchymal stromal cell is isolated from an adult source. In another example, the mesenchymal stromal cell is isolated from adult bone marrow. The mesenchymal stromal cells may be isolated from a living subject or a cadaver. In some instances, the Stro-1+ cells may be isolated from a source by cell sorting. In some examples, Stro-1+ cells may be identified and/or characterized based on biomarker expression, including by flow cytometry (such as FACS analysis), immunochemistry, or magnetic bead separation. Also, microarray and quantitative PCR characterization of biomarker expression may be used to identify expression profiles for SIPs, for example, by assessing the amount of protein, DNA, or mRNA transcripts of certain biomarkers. In one example, Stro-1+ cells can be isolated from heparinized adult human bone marrow using an anti-Stro-1+ antibody and magnetic cell sorting. In another example, isolated adult Stro-1+ cells can be cultured at low density using medium that support mesenchymal cell growth. Optionally, the cells can be cultured on 0.2% gelatin coated flasks.

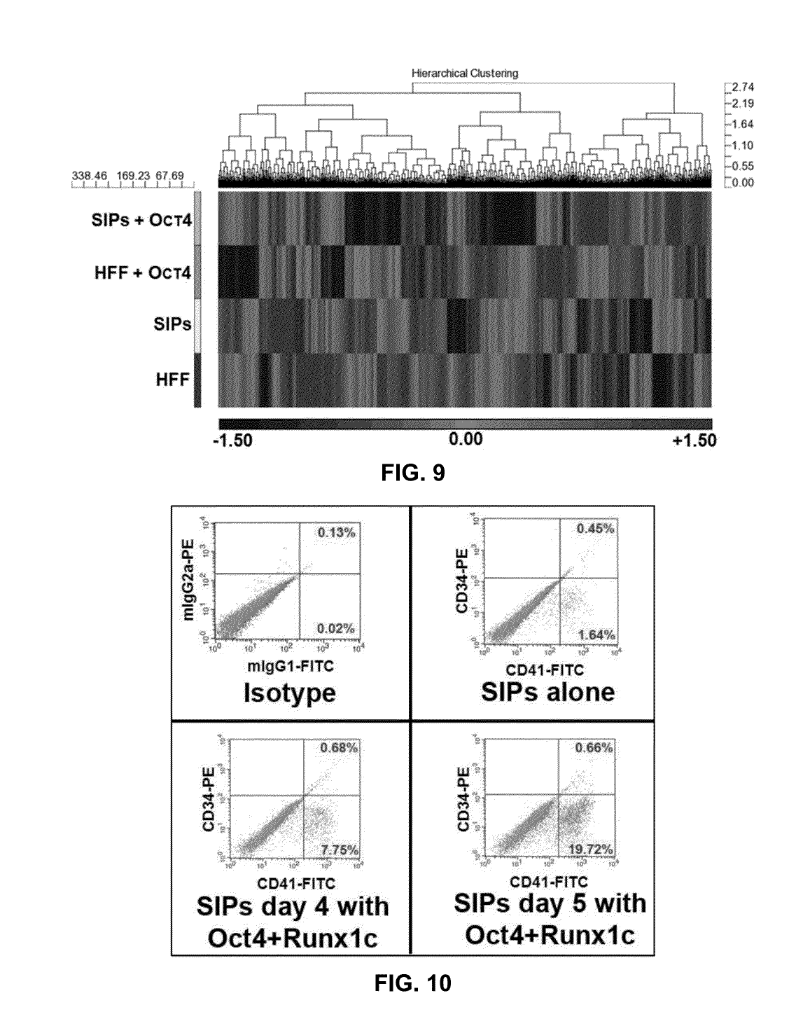

In certain instances, the mesenchymal stromal cell may express a characteristic pattern of biomarkers. In some instances, the mesenchymal stromal cell may express Stro1 but not express at least one of CD34, CD45, and GlyA. For example, the mesenchymal stromal cells may express Stro1 but not express CD34, CD45, or GlyA. An example is shown in FIGS. 3B and 3C. In some cases, the mesenchymal stromal cells may express APJ but may not express PDGFRα. An example is shown in FIG. 3G. In one example, quantitative real-time PCR analysis of mRNA expression, such as by using microarray analysis, may be performed to assess the transcriptional profile of the mesenchymal stromal cells (SIPs) as shown, for example, in FIG. 9. In one example, quantitative real time PCR may be performed to assess expression of biomarkers associated with hemangioblast stage and/or commitment to the hematopoietic lineage as listed in Table 1. In some instances, such analysis may identify an profile of hemangioblast- and hematopoiesis-associated gene expression associated with unmanipulated mesenchymal stromal cells (SIPs) as shown, for example, in Table 2. In certain instances, the mesenchymal stromal cells (SIPs) may express hemangioblast-associated biomarkers including, but not limited to, ANGPT1, KIT, MEIS1 and 2, NPR3, HEX, DLX5. In certain cases, the mesenchymal stromal cells may not express one or more of hemangioblast-associated markers CRHBP, GATA2, HLF, and PROM-1. In some instances, the mesenchymal stromal cells may express biomarkers including, but not limited to, hemangioblast-associated markers ANGPT1, KIT, MEIS1 and 2, NPR3, HEX, DLX5 and may not express hemangioblast-associated markers CRHBP, GATA2, HLF, and PROM-1. In some cases, the mesenchymal stromal cells may express at least one of hematopoiesis-associated markers SCL, RUNX1B, HHEX, KLF2, NFE2. In some cases, the mesenchymal stromal cells may not express hematopoiesis-associated markers RUNX1C, LYL-1, LMO-2, GATA-2, PU.1, C-MYC ERG, FLI-1, GFIB, MLL, HOXB4, and CDX4. In some instances, the mesenchymal stromal cells may express hematopoiesis-associated biomarkers including, but not limited to, SCL, RUNX1B, HHEX, KLF2, NFE2 and may not express hematopoiesis-associated markers RUNX1C, LYL-1, LMO-2, GATA-2, PU.1, C-MYC ERG, FLI-1, GFIB, MLL, HOXB4, and C_{DX}4. In certain cases, the mesenchymal cells may have low/undetectable levels of one or both of MBD2 and MBD3, components of the nucleosome remodeling and deacetylation (NuRD) complex.

In certain instances, mesenchymal stromal cells in adult bone marrow, or SIPs, may have the ability to undergo in vivo reprogramming to generate serially transplantable hematopoietic stem cells, for example, as described in Section A.1. In one example, SIPs can be propagated for up to about 30 population doublings in vitro without senescing and without exhibiting any signs of genetic instability or loss of potential (using, for example, methods described by Chamberlain et al. 2007). In some cases, adequate numbers of SIPs for reprogramming methods can be generated from a single marrow aspirate and used to produce substantial numbers of hematopoietic cells with the reprogramming efficiency described herein. In some cases, SIPs can be cultured under highly defined serum-free conditions.

In some instances, the isolated mesenchymal stromal cells, or SIPs, are transduced with a vector that encodes a transcription factor that controls hematopoietic cell differentiation. The vector may be an integrating vector or a non-integrating vector. For example, the vector may be a vector that integrates into the genome of transduced cells. In one example, the vector may be a lentivirus vector. A lentivirus vector may integrate into the genome of dividing or non-dividing cells. The lentivirus genome in the form of RNA is reverse-transcribed to DNA when the virus enters the cell, and is then inserted into the genome by the viral integrase enzyme. The lentivirus vector, now called a provirus, remains in the genome and is passed on to the progeny of the cell when it divides. In another example, the vector may be an adeno-associated virus (AAV) vector, which, in contrast to wild-type AAV, only rarely integrates into the genome of the cells its transduces. In one example, the vector may be an adenoviral vector. An adenoviral vector does not integrate into the genome. For example, adenovirus vectors have been used successfully to deliver TF for cellular reprogramming (see for example Zhou et al. 2009). In some instances, because adenoviruses may trigger an immune response, culturing the cells for a period of time after using adenoviral vectors for reprogramming would be advisable to ensure any residual adenoviral proteins have been lost from the cells prior to transplantation, to reduce the risk of immune response upon clinical use of reprogrammed hematopoietic cells generated from the isolated mesenchymal cells. In another instance, the vector may be a murine retrovirus vector. In another example, the vector may be a foamy virus vector. In another example, the vector may be Sendai virus vector. In some instances, the isolated mesenchymal stromal cells may be transduced by one vector. In other instances, the isolated mesenchymal stromal cells may be transduced by more than one vector. In certain cases, the isolated mesenchymal stromal cells may be transduced by more than one kind of vector. In one example, the vector may be a lentivirus vector, wherein the lentivirus vector may be generated by transfecting an expression cell line, such as 293T cells or like, with a lentiviral backbone plasmid and packaging constructs and, after culturing, viral vector constructs can be collected from the culture medium.

In certain cases, the transcription factor encoded by the vector may trigger the reprogramming of the mesenchymal stromal cell into a hematopoietic cell. For example, the transcription factor may activate expression of genes characteristic of hematopoietic cells. In some instances, the transcription factor may be at least one of RUNX1C or OCT4. For example, the transcription factor may be RUNX1C or OCT4. In one example, the transcription factor is RUNX1C. In another example, the transcription factor is OCT4. In another example, the transcription factor may be both RUNX1C and OCT4. In some instances, a cDNA coding sequence for the transcription factor may be cloned into a viral vector plasmid and used to generate the viral vector.

In some instances, the isolated mesenchymal stromal cells, or SIPs, include one or more vectors that encode a plurality of transcription factors that control hematopoietic cell differentiation. In some cases, the vector encodes a plurality of transcription factors. For example, the vector may include a nucleic acid sequence encoding RUNX1C and a nucleic acid sequence encoding OCT4. In other instances, the isolated mesenchymal stromal cell may include more than one vector. The vectors may each contain nucleic acid encoding a different transcription factor. For example, the isolated mesenchymal stromal cells may include a vector including a nucleic acid sequence encoding RUNX1C and a vector including a nucleic acid sequence encoding OCT4. Various combinations of transcription factors as noted in the preceding paragraph are contemplated.

In some instances, expression of the one or more transcription factors encoded by the one or more vectors can be regulated such that expression of the one or more transcription factors can be turned on or turned off depending on cell culture conditions. For example, a vector may contain a regulatable promoter operably linked to the nucleic acid sequence encoding a transcription factor. In another example, where the isolated mesenchymal stromal cell contains a vector having more than one nucleic acid encoding a transcription factor, each nucleic acid may be operably linked to the same regulatable promoter, either independently or combined. Alternatively, each nucleic acid may be operably linked to a different regulatable promoter. In some instances, only one of the nucleic acids encoding a transcription factors in the vector may be operably linked to a regulatable promoter. In another example, where the isolated mesenchymal stromal cell are transduced with more than one vector each having nucleic acid encoding a different transcription factor, each nucleic acid may be operably linked to the same regulatable promoter. Alternatively, each nucleic acid may be operable linked to a different regulatable promoter. In some instances, only one of the vectors may have a nucleic acid encoding a transcription factor may be operably linked to a regulatable promoter. In some instances, having the expression of different transcription factors controlled by different regulatable promoters may allow independent control of expression for each of the transcription factors. In one example, the isolated mesenchymal stromal cell may be transduced with a vector having a regulatable promoter operably linked to a nucleic acid sequence that encodes RUNX1C. In another example, the isolated mesenchymal stromal cell may be transduced with a vector having a regulatable promoter operably linked to a nucleic acid sequence that encodes OCT4. In another example, the isolated mesenchymal stromal cell may be transduced with a vector having a regulatable promoter operably linked to a nucleic acid sequence that encodes RUNX1C and OCT4. In other examples, the isolated mesenchymal stromal cell may be transduced with a vector having a regulatable promoter operably linked to a nucleic acid sequence that encodes RUNX1C and a vector having a regulatable promoter operably linked to a nucleic acid sequence that encodes OCT4, where the regulatable promoters may be the same promoter or different promoters. Various combinations of transcription factors as discussed above are also contemplated.

In some cases, the regulatable promoter may be a promoter that permits gene expression to be turned on or turned off by addition of an appropriate inducer or repressor. For example, no or little expression may occur in the absence of the inducer or in the presence of the repressor. In some instances, the inducible promoter system results in tight regulation of gene expression such that at most only a low level of the induced gene is expressed when the system is not induced. Many regulatable eukaryotic promoter systems are known and contemplated within the scope of this disclosure. Exemplary regulatable promoters include the tetracycline-inducible gene switch (TET) promoter (see, for example, Gossen and Bujard 1992; Gossen et al. 1995), TET-derivative systems such as the TET-regulated KRAB system (see, for example, Szulc et al., 2006), hormone-modulated systems (see, for example, Wang et al. 1994; Delort and Capecchi 1996; No et al. 1996), the mifepristone-inducible (GAL4) mammalian expression system (see, for example, Wang et al. 1994), metal-inducible promoters such as metallothionein promoter systems (see, for example, Makarov et al. 1994), and small molecule-modulated systems such as the rapamycin system (see, for example, Rivera et al. 1996) or the cumate gene switch (see, for example, Mullick et al. 2006). Other exemplary promoters include the TDH3, ADH1, TPI1, ACT1, GPD or PGI, or the galactose inducible promoters, GAL1, GAL7 and GAL10.

In some instances, the isolated mesenchymal stromal cell may be cultured in culture medium after transducing the isolated mesenchymal stromal cell with the vector. In some instances, after transduction, the mesenchymal stromal cell may be cultured for 1, 2, 3, 4, 5, 6, 7, 8, 9, 10, 11, 12, 13, 14, 15, 16, 17, 18, 19, or 20 days. For example, the mesenchymal stromal cell is cultured for at least 4 days. In one example, the mesenchymal stromal cell is cultured for 2 to 5 days, or 3 to 5 days, or 4 to 5 days, or 3 to 6 days, or 4 to 6 days. In one example, the mesenchymal stromal cell is cultured for 2 to 5 days. In another example, the mesenchymal stromal cell is cultured for 8 to 10 days, or about 9 days.

In some instances, expression of the transcription factor may occur immediately after transduction of the isolated mesenchymal cells. For example, the transcription factor may be continuously expressed once the mesenchymal cell has been transduced with the vector. In other instances, expression of the transcription factor may be induced by activating expression from a regulatable promoter operably linked to the nucleic acid encoding the transcription factor. In some instances, expression of the transcription factor may be induced prior to reprogramming of the mesenchymal stromal cell. In some examples, the method of generating a hematopoietic cell may further include activating expression of the transcription factor by culturing the transduced mesenchymal stromal cells in the presence of an inducer that activates transcription from the regulatable promoter. In other examples, the method may include activating expression of the transcription factor by culturing the transduced mesenchymal stromal cells in the absence of a repressor that represses transcription from the regulatable promoter. In some examples, expression of the transcription factor may not occur when the mesenchymal stromal cells are cultured in the absence of an inducer that activates transcription from the regulatable promoter. In some examples, expression of the transcription factor may not occur when the mesenchymal stromal cells are cultured in the presence of a repressor that represses transcription from the regulatable promoter. In some instances, expression of the one or more transcription factors is not required after reprogramming of the isolated mesenchymal stromal cells along the hematopoietic lineage. In some instances, expression of the one or more transcription factors may be reduced or prevented upon reprogramming of the isolated mesenchymal stromal cells along the hematopoietic lineage. In some instances, expression of the transcription factor may be induced for 1, 2, 3, 4, 5, 6, 7, 8, 9, 10, 11, 12, 13, 14, 15, 16, 17, 18, 19, or 20 days. For example, the expression of the transcription factor may be induced for 2 to 5 days, or 3 to 5 days, or 4 to 5 days, or 3 to 6 days, or 4 to 6 days. In one example the transcription factor expression may be induced for 2 to 5 days.

In some instances, the isolated mesenchymal cell may be transduced with the vector and then cultured in a culture medium that includes a compound that alters chromatin modification. For example, the compound that alters chromatin modification may be a chromatic-modifying enzyme inhibitor such as a histone methyltransferase inhibitor or a histone deacetylase (HDAC) inhibitor. In some instances, the transduced mesenchymal cell may be cultured in the presence of at least one histone methyltransferase inhibitor and at least one histone deacetylase (HDAC) inhibitor. Without being held to any particular theory, such inhibitors may assist in reprogramming of the isolated mesenchymal cell by modifying their epigenetic profile. For example, DNA methylation and histone modifications constitute major mechanisms that are responsible for epigenetic regulation of gene expression during development and differentiation. Both DNA methylation and histones deacetylation are characteristic of inactive (untranscribed) regions of the genome. Exemplary histone methyltransferase inhibitors include compounds such as, but not limited to, Bix-01294, UNC0638, BRDD4770, EPZ004777, AZ505, and PDB4e47. In one example, the histone methyltransferase inhibitor may be Bix-01294. Exemplary histone deacetylase inhibitors include compounds such as, but not limited to, valproic acid, vorinostat, romidepsin, entinostat abexinostat, givinostat, and mocetinostat, butyrate, or a serine protease inhibitor (serpin) family member. In one example, the histone deacetylase inhibitor may be valproic acid. In one example, the transduced mesenchymal cell may be cultured in the presence of Bix-01294 and valproic acid. The concentration of chromatin-modifying enzyme inhibitor in the culture media used to culture the transduced mesenchymal cells may depend on the inhibitor. In some instances, the concentration of Bix-01294 with which the cells are cultured may range from 0.1-10 μM and, in some instances, may range from 0.1-1 μM, 0.5-4.0 μM, 3.0-7.5 μM, 5.0-8.0 μM, or 7.5-10 μM. In some instances, the concentration of valproic acid with which the cells are cultured may range from 0.1-10 μM and, in some instances, may range from 0.1-1 μM, 0.5-4.0 μM, 3.0-7.5 μM, 5.0-8.0 μM, or 7.5-10 μM.

In certain instances, the method of generating a hematopoietic cell may further include harvesting a cell population enriched for hematopoietic cells. In some instances, the cell population enriched for hematopoietic cells may be harvested after culturing the transduced mesenchymal cell. In some cases, the harvesting may occur after expression of the transcription factor has been induced. In some instances, the cell population enriched for hematopoietic cells may include one or more of hematopoietic stem cells (HSC), hematopoietic progenitor cells (HPC), myeloid progenitor cells (MPC), lymphoid progenitor cells (LPC), lymphocytes, granulocytes, macrophages, erythrocytes, platelets, neutrophils, or natural kill cells. For example, the cell population enriched for hematopoietic cells may include hematopoietic stem cells (HSC). In some instances, the cell population enriched for hematopoietic cells may include one or more of a hematopoietic progenitor cell having the ability to differentiate into at least one of myeloid lineage, lymphoid lineage, megakaryocyte lineage, or erythroid lineage. In certain instances, depending on the culture conditions selected for the transduced mesenchymal cells, the cell population may be enriched for different types of hematopoietic cells. In some instances, the cell population enriched for hematopoietic cells may include one or more of a hematopoietic progenitor cell having the ability to differentiate into at least one of myeloid lineage, lymphoid lineage, megakaryocyte lineage, or erythroid lineage. In certain instances, the cell population enriched for hematopoietic cells may include a plurality of different types of hematopoietic cells. In some cases, the hematopoietic cells may be partially or fully differentiated hematopoietic cells. For example, culture conditions may be selected to result in differentiation of the hematopoietic cells along different maturation pathways.

In one example, SIPs may be transduced with a lentiviral vector encoding the OCT4 gene and cultured in vitro. In certain instances, SIPs isolated from adult bone marrow may be transduced with a lentivirus vector encoding RUNX1C and a lentivirus vector encoding OCT4 at a multiplicity of infection (MOI) of about 50 for each vector by culturing the SIPs in viral vector containing medium for 48 hours. In certain instances, SIPs isolated from adult bone marrow may be transduced with a lentivirus vector encoding either OCT4 or RUNX1C at a multiplicity of infection (MOI) of about 50 by culturing the SIPs in viral vector containing medium for 48 hours. In some instances, the transduced SIPS may be cultured in culture medium supplemented with IGF-II and bFGF, for example, at concentrations of about 30 μg/ml and about 20 μg/ml, respectively. In certain cases, such as where the SIPs have been transduced with a viral vector encoding RUNX1C alone, the medium may be supplemented with a compound that alters chromatin modification such as Bix-01294 and valproic acid, at concentrations of about 1-5 μM and about 0.5-2.5 mM, respectively.

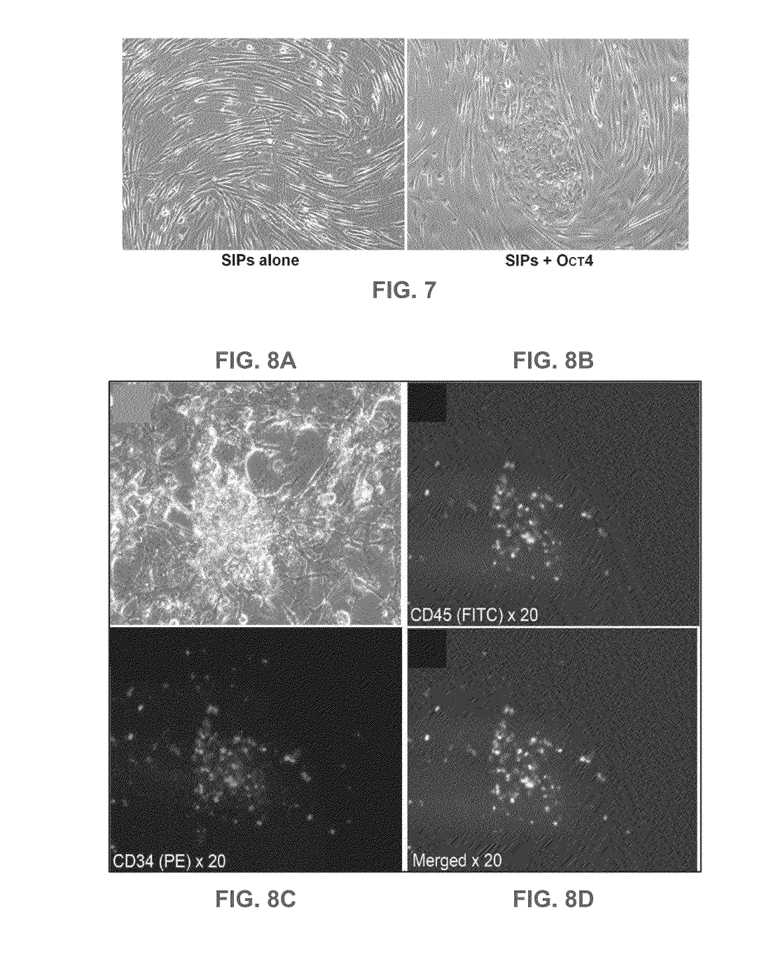

In some instances, SIPs expressing exogenous OCT4 from a viral vector may reprogram within about 9-10 days in culture in vitro as shown, for example, in FIG. 7. In comparison, human dermal fibroblasts (HFF) expressing exogenous OCT4 from a viral vector may reprogram 2-3 times slower and with less efficiency after 22-27 days in culture as shown, for example, in FIG. 7. In some cases, the reprogramming occurs with greater efficiency than has been reported for HFF (see Szabo et al. 2010). In some instances, SIPs transduced with OCT4 and cultured in vitro express CD45 and CD34, early hematopoietic cell markers as shown, for example, in FIG. 8A-8D.

In certain cases, transcriptional profiling of untransduced mesenchymal stromal cells and mesenchymal stromal cells transduced with a vector encoding OCT4 may be performed using a microarray analysis assessing biomarker mRNA expression levels. Exemplary expression profiles based on microarray analysis are illustrated by the heatmap shown in FIG. 9. In some instances, there is a quantifiable difference between the expression profiles of untransduced mesenchymal stromal cells and mesenchymal stromal cells transduced with a vector encoding OCT4, as well as between transduced mesenchymal cells and HFF cells transduced with a vector encoding OCT4.

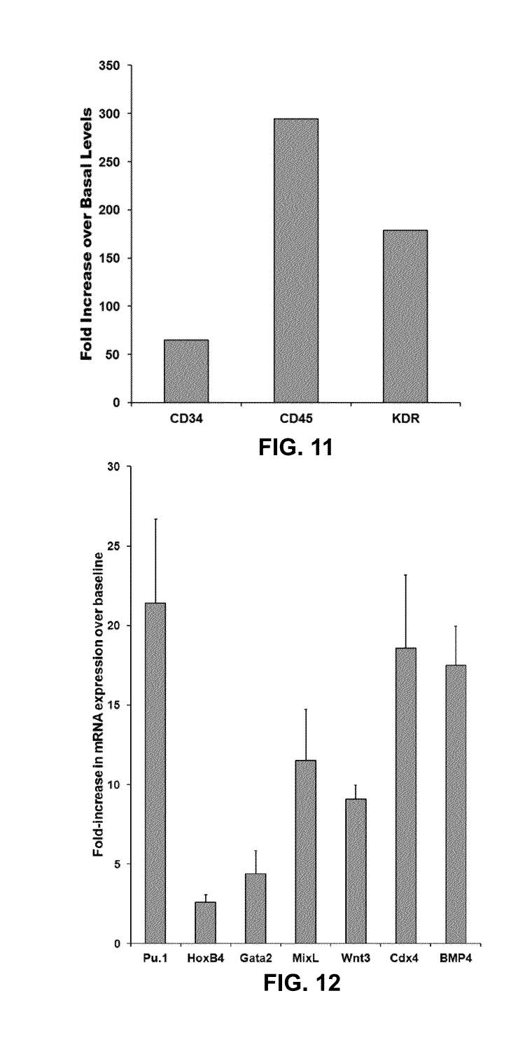

In one aspect, transcription factors involved in hematopoietic emergence/fate specification whose expression is lacking in SIPs may be exogenously expressed, such as by a viral vector, to enhance the speed and efficiency of reprogramming of SIPs to the hematopoietic lineage in vitro. For example, exogenous expression of RUNX1C, such as from a viral vector, can drive efficient reprogramming of SIPs to the hematopoietic lineage. In another example, exogenous expression of OCT4 and RUNX1C in SIPs results in expression of CD41 within only 3-4 days as shown, for example, in FIG. 10. In another example, exogenous expression of OCT4 and RUNX1C in SIPs results in peak CD41 expression at day 5-6 after transduction as shown, for example, in FIG. 11. In some instances, ˜20% of SIPs expressed CD41. In some examples, peak CD41 expression at day 5-6 of culturing coincided with commencement of expression of the phenotypic markers CD34, CD45, and KDR (see, for example, FIG. 11), and maximal induction of several hematopoiesis-specific transcription factors including, but not limited to, Pu.1, HOXB4, Gata2, MixL, Wnt3, Cdx4, and BMP (see, for example, FIG. 12). In some instances, the levels of hematopoietic transcription factors and phenotypic markers in mesenchymal stromal cells exogenously expressing OCT4 and RUNX1C from a viral vector may be 1-3 logs higher than the levels in similarly transduced HFF cells.

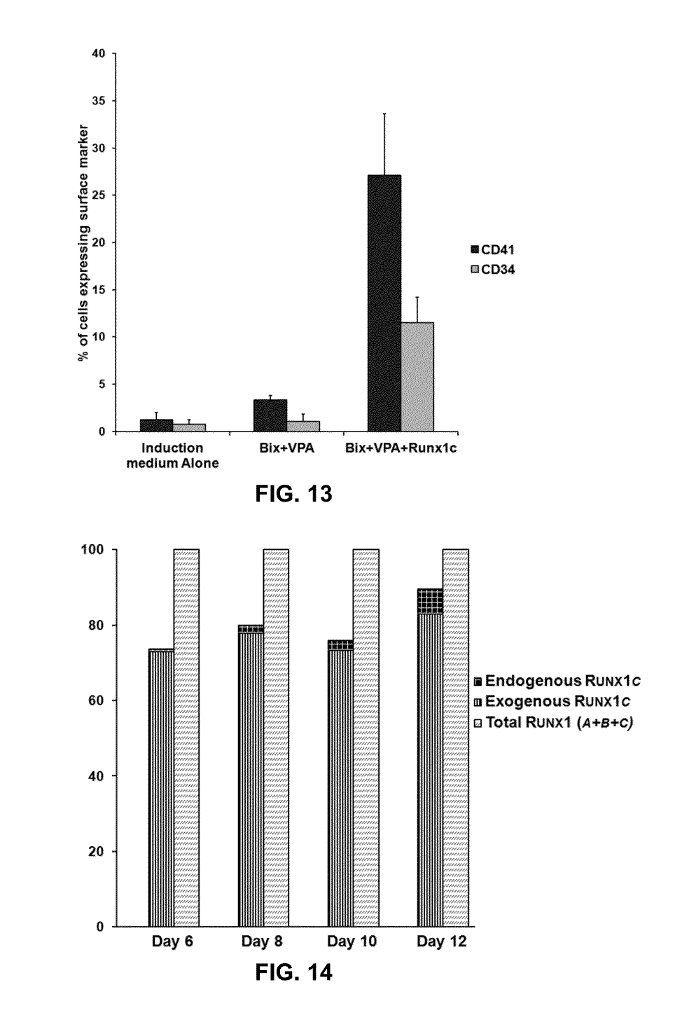

In certain cases, SIPs can be transduced with RUNX1C and incubated with chromatin remodeling agents to cause reprogramming into hematopoietic cells. Exemplary chromatin remodeling agents include compounds such as Bix-01294 and VPA. In some cases, ˜35% and ˜10% of SIPs can be reprogrammed to express CD41 and CD34 within a 4-5 day period, respectively, as shown, for example, in FIG. 13. CD41 expression generally marks the onset of primitive/definitive hematopoiesis (see Ferkowicz et al. 2003, Mikkola et al. 2003, Robin et al. 2011). CD41 is the product of the ITGA2B gene, and its expression is generally associated with the earliest stages of hematopoiesis, most notably on cells of the intra-aortic hematopoietic clusters, which is where the first adult-repopulating HSC are generated. In some cases, expression of CD41 in reprogrammed SIPs may be followed by expression of other definitive hematopoietic markers such as CD34, CD45, KDR, and CD49f. In some examples, the reprogramming of SIPs induced by in vitro expression of RUNX1C may proceed along development pathways similar to those described during normal ontogeny/embryogenesis (as described by McKinney-Freeman et al. 2009).

In another example, expression of endogenous RUNX1C and exogenous RUNX1C expressed from the viral vector used to transduce the mesenchymal stromal cells may be assessed by quantitative real-time PCR. In some instances, exogenous expression of RUNX1C may inhibit expression of endogenous RUNX1C as shown, for example, in FIG. 14. In some instances, where the promoter operatively coupled to the RUNX1C coding sequence in the viral vector is constitutively active, such as the EF1α promoter, expression of the exogenous RUNX1C may persist at high levels even at 12 days post transduction.

In some instances, the ability to generate hematopoietic stem/progenitor cells (HSC) by the reprogramming of mature somatic cells may provide the capability to produce therapeutically sufficient numbers of corrected autologous or matched allogeneic HSC to treat/cure a broad variety of blood diseases. For discussion of certain diseases see, for example, Hama et al. 2007, Hexum et al. 2011, Sakamoto et al. 2010, Szabo et al. 2010, Woods et al. 2011, Ye et al. 2013, Zou et al. 2011. In some instances, the hematopoietic cells reprogrammed from stromal cells are an improvement over hematopoietic cells generated in vitro via iPSC technology, which have not proven to be fully mature/functional cells capable of durably engrafting all blood lineages of an adult recipient. Hematopoietic cells generated in vitro via iPSC technology also raise clinical concerns regarding the safety/stability of cells generated through an iPSC intermediate derived via forced overexpression of pluripotency transcription factors.

In certain instances, the method of reprogramming the mesenchymal stromal cells described herein may bypass the need for a pluripotent intermediate by achieving direct reprogramming of somatic cells to the hematopoietic lineage. By starting with SIPs, a cell type that shares developmental origin with HSCs, the efficiency and speed of direct reprogramming to the hematopoietic lineage may be significantly improved. This is in contrast, for example, to reprogramming of HFFs to hematopoietic progenitors in which expression of OCT4 alone was sufficient to reprogram HFFs but the process took several weeks and was relatively inefficient as compared to the methods described herein (see Szabo et al. 2010).

3. Cellular Compositions

In one aspect, isolated mesenchymal stromal cells are provided that include a vector that includes a nucleic acid sequence encoding a transcription factor that controls hematopoietic cell differentiation. In one example, the vector permits in vitro reprogramming of the isolated mesenchymal cells into hematopoietic cells.

In various aspects, the isolated mesenchymal stromal cell may have characteristics and properties as set forth in Section A.1. Mesenchymal stromal cells may be isolated from various sources. Exemplary sources include, for example, bone marrow, adipose tissue, brain, liver, lung, intestine, dental pulp, and umbilical cord tissue. In one example, the mesenchymal stromal cells may be isolated from bone marrow. The mesenchymal stromal cells may be isolated from fetal or adult sources. In one example, the mesenchymal stromal cell is isolated from an adult source. In another example, the mesenchymal stromal cell is isolated from adult bone marrow. The mesenchymal stromal cells may be isolated from a living subject or a cadaver. In some instances, the Stro-1+ cells may be isolated from a source by cell sorting. In some examples, Stro-1+ cells may be identified and/or characterized based on biomarker expression, including by flow cytometry (such as FACS analysis), immunochemistry, or magnetic bead separation. Also, microarray and quantitative PCR characterization of biomarker expression may be used to identify expression profiles for SIPs, for example, by assessing the amount of protein, DNA, or mRNA transcripts of certain biomarkers. In one example, Stro-1+ cells can be isolated from heparinized adult human bone marrow using an anti-Stro-1+ antibody and magnetic cell sorting. In another example, isolated adult Stro-1+ cells can be cultured at low density using medium that support mesenchymal cell growth. Optionally, the cells can be cultured on 0.2% gelatin coated flasks.

In certain instances, the mesenchymal stromal cell may express a characteristic pattern of biomarkers. In some instances, the mesenchymal stromal cell may express Stro1 but not express at least one of CD34, CD45, and GlyA. For example, the mesenchymal stromal cells may express Stro1 but not express CD34, CD45, or GlyA. An example is shown in FIGS. 3B and 3C. In some cases, the mesenchymal stromal cells may express APJ but may not express PDGFRα. An example is shown in FIG. 3G. In one example, microarray analysis of mRNA expression may be performed to assess the transcriptional profile of the mesenchymal stromal cells as shown, for example, in FIG. 9. In some instances, microarray analysis may identify an expression profile of hemangioblast- and hematopoiesis-associated gene expression associated with the mesenchymal stromal cells as shown, for example, in Table 2. In certain instances, the mesenchymal stromal cells (SIPs) may express biomarkers including, but not limited to, ANGPT1, KIT, MEIS1 and 2, NPR3, HEX, DLX5. In certain cases, the mesenchymal stromal cells may not express one or more of CRHBP, GATA2, HLF, and PROM-1. For example, the mesenchymal stromal cells may express biomarkers including, but not limited to, ANGPT1, KIT, MEIS1 and 2, NPR3, HEX, DLX5 and may not express CRHBP, GATA2, HLF, and PROM-1. In some cases, the mesenchymal stromal cells may express at least one of SCL, RUNX1B, HHEX, KLF2, NFE2. In some cases, the mesenchymal stromal cells may not express RUNX1C, LYL-1, LMO-2, GATA-2, PU.1, C-MYC ERG, FLI-1, GFIB, MLL, HOXB4, and C_{DX}4. For example, the mesenchymal stromal cells may express biomarkers including, but not limited to, SCL, RUNX1B, HHEX, KLF2, NFE2 and may not express RUNX1C, LYL-1, LMO-2, GATA-2, PU.1, C-MYC ERG, FLI-1, GFIB, MLL, HOXB4, and C_{DX}4. In certain cases, the mesenchymal cells may have low/undetectable levels of one or both of MBD2 and MBD3, components of the nucleosome remodeling and deacetylation (NuRD) complex.

In another aspect, the isolated mesenchymal stromal cells, or SIPs, contain a vector that encodes a transcription factor that controls hematopoietic cell differentiation. The vector may be an integrating vector or a non-integrating vector as discussed above in Section A.2. For example, the vector may be an integrating vector that integrates into the genome of transduced cells. In one example, the vector may be a lentivirus vector. In another example, the vector may be an adeno-associated virus (AAV) vector. In one example, the vector may be an adenoviral vector. In another example, the vector may be a murine retrovirus vector. In another example, the vector may be a foamy virus vector. In another example, the vector may be Sendai virus vector. In some instances, the isolated mesenchymal stromal cells may be transduced by one vector. In other instances, the isolated mesenchymal stromal cells may be transduced by more than one vector. In certain cases, the isolated mesenchymal stromal cells may be transduced by more than one kind of vector. In one example, the vector may be a lentivirus vector, wherein the lentivirus vector may be generated by transfecting an expression cell line, such as 293T cells or like, with a lentiviral backbone plasmid and packaging constructs and, after culturing, viral vector constructs can be collected from the culture medium.

In certain cases, the transcription factor encoded by the vector may have the ability to trigger the reprogramming of the mesenchymal stromal cell into a hematopoietic cell when expressed. For example, the transcription factor may activate expression of genes characteristic of hematopoietic cells. In certain cases, the transcription factor encoded by the vector may be at least one of RUNX1C or OCT4. In one example, the transcription factor is RUNX1C. In another example, the transcription factor is OCT4. In another example, the transcription factor is both RUNX1C and OCT4. In some instances, a cDNA coding sequence for the transcription factor may be cloned into a viral vector plasmid and used to generate the viral vector.

In some instances, the isolated mesenchymal stromal cells, or SIPs, contain one or more vectors that encodes a plurality of transcription factors that control hematopoietic cell differentiation. In some cases, the isolated mesenchymal stromal cells may contain one vector that encodes a plurality of transcription factors. For example, the vector may include a nucleic acid sequence encoding RUNX1C and a nucleic acid sequence encoding OCT4. In other instances, the isolated mesenchymal stromal cell may contain more than one vector. The vectors may each contain a nucleic acid encoding a different transcription factor. For example, one vector may include a nucleic acid sequence encoding RUNX1C and another vector may include a nucleic acid sequence encoding OCT4. Various combinations of transcription factors as noted in the preceding paragraph are contemplated.

In some instances, expression of the one or more transcription factors encoded by the one or more vectors can be regulated such that expression of the one or more transcription factors can be turned on or turned off depending on cell culture conditions. For example, a vector may contain a regulatable promoter operably linked to the nucleic acid sequence encoding a transcription factor. In another example, where the isolated mesenchymal stromal cell contains a vector having more than one nucleic acid encoding a transcription factor, each nucleic acid may be operably linked to the same regulatable promoter, either independently or combined. Alternatively, each nucleic acid may be operably linked to a different regulatable promoter. In some instances, only one of the nucleic acids encoding a transcription factors in the vector may be operably linked to a regulatable promoter. In another example, where the isolated mesenchymal stromal cell includes more than one vector each having nucleic acid encoding a different transcription factor, each nucleic acid may be operably linked to the same regulatable promoter. Alternatively, each nucleic acid may be operable linked to a different regulatable promoter. In some instances, only one of the vectors may have a nucleic acid encoding a transcription factor may be operably linked to a regulatable promoter. In some instances, having the expression of different transcription factors controlled by different regulatable promoters may allow independent control of expression for each of the transcription factors. In one example, the isolated mesenchymal stromal cell may include a vector having a regulatable promoter operably linked to a nucleic acid sequence that encodes RUNX1C. In another example, the isolated mesenchymal stromal cell may include a vector having a regulatable promoter operably linked to a nucleic acid sequence that encodes OCT4. In another example, the isolated mesenchymal stromal cell may include a vector having a regulatable promoter operably linked to a nucleic acid sequence that encodes RUNX1X and OCT4. In other examples, the isolated mesenchymal stromal cell may include a vector having a regulatable promoter operably linked to a nucleic acid sequence that encodes RUNX1C and a vector having a regulatable promoter operably linked to a nucleic acid sequence that encodes OCT4, where the regulatable promoters may be the same promoter or different promoters. Various combinations of transcription factors as discussed above are also contemplated.

In some cases, the regulatable promoter may be a promoter that permits gene expression to be turned on or turned off by addition of an appropriate inducer or repressor. For example, no or little expression may occur in the absence of the inducer or in the presence of the repressor. In some instances, the inducible promoter system results in tight regulation of gene expression such that at most only a low level of the induced gene is expressed when the system is not induced. Many regulatable eukaryotic promoter systems are known and contemplated within the scope of this disclosure, and examples provided as discussed above in Section A.1. Exemplary regulatable promoters include the tetracycline-inducible gene switch (TET) promoter, TET-derivative systems such as the TET-regulated KRAB system, hormone-modulated systems, the mifepristone-inducible (GAL4) mammalian expression system, metal-inducible promoters such as metallothionein promoter systems, and small molecule-modulated systems such as the rapamycin system or the cumate gene switch. Other exemplary promoters include the TDH3, ADH1, TPI1, ACT1, GPD or PGI, or the galactose inducible promoters, GAL1, GAL7 and GAL10.

In some instances, the one or more transcription factors from the one or more vectors may be continuously expressed in the mesenchymal cell. In other instances, expression of the transcription factor may be induced by activating expression from an regulatable promoter operably linked to the nucleic acid encoding the transcription factor. In some instances, expression of the transcription factor may be induced prior to reprogramming of the mesenchymal stromal cell. In some examples, expression of the transcription factor may occur when the mesenchymal stromal cells are cultured in the presence of an inducer that activates transcription from the regulatable promoter. In other examples, expression of the transcription factor may occur when the mesenchymal stromal cells are cultured in the absence of a repressor that represses transcription from the regulatable promoter. In some examples, expression of the transcription factor may not occur when the mesenchymal stromal cells are cultured in the absence of an inducer that activates transcription from the regulatable promoter. In some examples, expression of the transcription factor may not occur when the mesenchymal stromal cells are cultured in the presence of a repressor that represses transcription from the regulatable promoter. In some instances, the mesenchymal stromal cells may not express the one or more transcription factors until cultured in culture conditions that permit expression from a regulatable promoter. In some cases, expression of the one or more transcription factors triggers reprogramming of the isolated mesenchymal stromal cells along the hematopoietic lineage. In some instances, expression of the one or more transcription factors may be reduced or prevented upon reprogramming of the isolated mesenchymal stromal cells along the hematopoietic lineage.

In another aspect, hematopoietic cells are provided that include a vector that includes a nucleic acid sequence encoding a transcription factor that controls hematopoietic cell differentiation. In some instances, the hematopoietic cell may be a reprogrammed from an isolated mesenchymal stromal cell that has been transduced with the vector as described above in Section A.1. In various aspects, the isolated mesenchymal stromal cell may have characteristics and properties as set forth in Section A.1. In some instances, the hematopoietic cell may be reprogrammed from a mesenchymal stromal cell that expresses Stro1 but does not express CD34, CD45, and GlyA.

In some instances, the hematopoietic cells reprogrammed from stromal cells are an improvement over hematopoietic cells generated in vitro via iPSC technology, which have not proven to be fully mature/functional cells capable of durably engrafting all blood lineages of an adult recipient. Hematopoietic cells generated in vitro via iPSC technology also raise clinical concerns regarding the safety/stability of cells generated through an iPSC intermediate derived via forced overexpression of pluripotency transcription factors.

Various types of hematopoietic cells are contemplated within the scope of this disclosure. In certain instances, the hematopoietic cell may one or more of hematopoietic stem cells (HSC), hematopoietic progenitor cells (HPC), myeloid progenitor cells (MPC), lymphoid progenitor cells (LPC), lymphocytes, granulocytes, macrophages, erythrocytes, platelets, neutrophils, or natural kill cells. For example, the hematopoietic cells may be hematopoietic stem cells (HSC). In some instances, the hematopoietic cells may be one or more of a hematopoietic progenitor cell having the ability to differentiate into at least one of myeloid lineage, lymphoid lineage, megakaryocyte lineage, or erythroid lineage. In certain instances, the hematopoietic cells may be a plurality of different types of hematopoietic cells. In some cases, the hematopoietic cells may be partially or fully differentiated hematopoietic cells.

In another aspect, the hematopoietic cells contain a vector that encodes a transcription factor that controls hematopoietic cell differentiation. The vector may be an integrating vector or a non-integrating vector as discussed above in Sections A.1 and A.2. For example, the vector may be an integrating vector that integrates into the genome of transduced cells. In one example, the vector may be a lentivirus vector. In another example, the vector may be a murine retrovirus vector. In another example, the vector may be a foamy virus vector. In another example, the vector may be an adeno-associated virus (AAV) vector. In one example, the vector may be an adenoviral vector. In another example, the vector may be Sendai virus vector.

In certain cases, the transcription factor encoded by the vector may be at least one of RUNX1C or OCT4. In one example, the transcription factor is RUNX1C. In another example, the transcription factor is OCT4. In another example, the transcription factor is both RUNX1C and OCT4.

In some instances, the hematopoietic cell may contain one or more vectors that encodes a plurality of transcription factors that control hematopoietic cell differentiation. In some cases, the hematopoietic cell may contain one vector that encodes a plurality of transcription factors. For example, the vector may include a nucleic acid sequence encoding RUNX1C and a nucleic acid sequence encoding OCT4. In other instances, the hematopoietic cell may contain more than one vector. The vectors may each contain a nucleic acid encoding a different transcription factor. For example, one vector may include a nucleic acid sequence encoding RUNX1C and another vector may include a nucleic acid sequence encoding OCT4. Various combinations of transcription factors as noted in the preceding paragraph are contemplated.

In some instances, expression of the one or more transcription factors encoded by the one or more vectors can be regulated such that expression of the one or more transcription factors can be turned on or turned off depending on cell culture conditions. For example, a vector may contain a regulatable promoter operably linked to the nucleic acid sequence encoding a transcription factor. In another example, where the isolated mesenchymal stromal cell contains a vector having more than one nucleic acid encoding a transcription factor, each nucleic acid may be operably linked to the same regulatable promoter, either independently or combined. Alternatively, each nucleic acid may be operably linked to a different regulatable promoter. In some instances, only one of the nucleic acids encoding a transcription factors in the vector may be operably linked to a regulatable promoter. In another example, where the hematopoietic cell includes more than one vector each having nucleic acid encoding a different transcription factor, each nucleic acid may be operably linked to the same regulatable promoter. Alternatively, each nucleic acid may be operable linked to a different regulatable promoter. In some instances, only one of the vectors may have a nucleic acid encoding a transcription factor may be operably linked to a regulatable promoter. In some instances, having the expression of different transcription factors controlled by different regulatable promoters may allow independent control of expression for each of the transcription factors. In one example, the hematopoietic cell may include a vector having a regulatable promoter operably linked to a nucleic acid sequence that encodes RUNX1C. In another example, the hematopoietic cell may include a vector having a regulatable promoter operably linked to a nucleic acid sequence that encodes OCT4. In another example, the hematopoietic cell may include a vector having a regulatable promoter operably linked to a nucleic acid sequence that encodes RUNX1C and OCT4. In other examples, the hematopoietic cell may include a vector having a regulatable promoter operably linked to a nucleic acid sequence that encodes RUNX1C and a vector having a regulatable promoter operably linked to a nucleic acid sequence that encodes OCT4, where the regulatable promoters may be the same promoter or different promoters. Various combinations of transcription factors as discussed above are also contemplated.

In some cases, the regulatable promoter may be a promoter that permits gene expression to be turned on or turned off by addition of an appropriate inducer or repressor. For example, no or little expression may occur in the absence of the inducer or in the presence of the repressor. In some instances, the inducible promoter system results in tight regulation of gene expression such that at most only a low level of the induced gene is expressed when the system is not induced. Many regulatable eukaryotic promoter systems are known and contemplated within the scope of this disclosure, and examples provided as discussed above in Sections A.1 and A.2. Exemplary regulatable promoters include the tetracycline-inducible gene switch (TET) promoter, TET-derivative systems such as the TET-regulated KRAB system, hormone-modulated systems, the mifepristone-inducible (GAL4) mammalian expression system, metal-inducible promoters such as metallothionein promoter systems, and small molecule-modulated systems such as the rapamycin system or the cumate gene switch. Other exemplary promoters include the TDH3, ADH1, TPI1, ACT1, GPD or PGI, or the galactose inducible promoters, GAL1, GAL7 and GAL10.

In some instances, the one or more transcription factors may not be expressed in the hematopoietic cells. For example, the hematopoietic cells may be cultured in the absence of an inducer that activates transcription from a regulatable promoter that is operably linked to the nucleic acid that encodes the one or more transcription factors. In another example, the hematopoietic cells may be cultured in the presence of a repressor that represses transcription from a regulatable promoter that is operably linked to the nucleic acid that encodes the one or more transcription factors.

In some instances, the one or more transcription factors from the one or more vectors may not be expressed in the hematopoietic cell. In other instances, expression of the transcription factor may be prevented by inactivating expression from an inducible promoter, or repressing a repressible promoter, operably linked to the nucleic acid encoding the transcription factor. In some examples, expression of the transcription factor may occur when the hematopoietic cells are cultured in the presence of an inducer that activates transcription from the regulatable promoter. In other examples, expression of the transcription factor may occur when the hematopoietic cells are cultured in the absence of a repressor that represses transcription from the regulatable promoter. In some examples, expression of the transcription factor may not occur when the hematopoietic cells are cultured in the absence of an inducer that activates transcription from the regulatable promoter. In some examples, expression of the transcription factor may not occur when the hematopoietic cells are cultured in the presence of a repressor that represses transcription from the regulatable promoter. In some instances, the hematopoietic cells may not express the one or more transcription factors when cultured in culture conditions that do not permit expression from a regulatable promoter. In some cases, expression of the one or more transcription factors may be inactivated at a stage of differentiation of the hematopoietic cells along the hematopoietic lineage. In some instances, expression of the one or more transcription factors may be reduced or prevented at a stage of differentiation of the hematopoietic cells along the hematopoietic lineage.

In some instances, the cell compositions described in this section may be cryopreserved. For example, the cell compositions may be exposed to a cryopreservative agent or cyroprotectant such as, for example, DMSO at a concentration of 5%, 10%, 15%, 20% and the like. The cell compositions may be exposed to the cryopreservative agent or cyroprotectant for only a brief amount of time, such as, for example, about 30 seconds or less, about 20 seconds or less, about 10 seconds or less, about 5 seconds or less, about 4 seconds or less, about 3 seconds or less, about 2 seconds, or about 1 second. In some instances, the cell compositions may then be rinsed and drained such that only a small amount of the cryopreservative agent or cyroprotectant remains associated with the cells (for example, less than 50 ppm). Typically, the amount of the cryopreservative agent or cyroprotectant remaining with a cell composition is present at a level that is acceptable for subsequent injection of or administration of the cell composition to a subject. In some instances, the cell compositions may be frozen. For example, the cell compositions may be exposed to a temperature of about −80° C. (such as in liquid nitrogen).

In some instances, the cell compositions described in this section may be packaged in a suitable container or package until use. The container or packaging may be suitable for storage at a temperature of about −80° C. and amendable to thawing. In some instances, when a cell composition is selected for use, it can be thawed and then administered.

B. Methods of Treatment

Disclosed are methods of treating a subject having a diminished hematopoietic cell population using the compositions described herein. For example, the subject may have a defective or deficient hematopoietic system or a hematopoietic system disease. In one aspect, the methods include identifying a subject having diminished hematopoietic cells (such as a subject with a hematopoietic defect, deficit or disease), providing hematopoietic cells reprogrammed including a vector that includes a nucleic acid encoding a transcription factor that controls hematopoietic cell differentiation, and administering the hematopoietic cells to the subject to repopulate the subject with the hematopoietic cell population. For example, repopulating the subject with hematopoietic cells may be engraftment of the transplanted (administered) cells in the subject. In some instances, the hematopoietic cells may have the characteristics and properties as described above in Section A.2 and A.3. In some instances, the hematopoietic cells may be reprogrammed from isolated mesenchymal cells having the characteristics and properties as described above in Section A.1-A.3. In certain instances, the hematopoietic cells may be reprogrammed from isolated mesenchymal cells as described above in Section A.1. In some instances, the hematopoietic cells are generated and used for treatment without intermediate cryopreservation. In some instances, the hematopoietic cells are cryopreserved and must be thawed prior to use for treatment.

In some instances, the subject has a disorder in which native hematopoietic cells populations are diseased, diminished in number, or exhibit reduced functionality. As described throughout, the disorder can be a disorder in which a subject's hematopoietic production or function is diminished or compromised globally, or in specific hematopoietic lineages, such as for example, disorders of red blood cells such as thalassemia or sickle cell anemia, disorders in platelets, disorders in immune cell function such as severe combined immune-deficiency (SCID). The disorder could also include aplastic anemia, Fanconi anemia, and any of the other genetic or acquired diseases that alter blood cell production, and could also include any of the myriad malignancies that affect the hematopoietic system, such as acute myeloid leukemia, chronic myeloid leukemia, acute lymphoblastic leukemia, Hodgkin lymphoma, non-Hodgkin lymphoma, or myelodysplastic syndromes (MDS). The reprogrammed hematopoietic cells could also be used in patients whose own hematopoietic cells were damaged or depleted by radiotherapy or chemotherapy to treat neuroblastoma, Ewing's sarcoma, gliomas, breast cancer, or other solid tumors. Any disease that could be treated by a bone marrow or hematopoietic stem cell transplant would be amenable to treatment using the hematopoietic cells generated by reprogramming SIPs.

Various types of hematopoietic cells are contemplated within the scope of this disclosure. In certain instances, the hematopoietic cell may be one or more of hematopoietic stem cells (HSC), hematopoietic progenitor cells (HPC), myeloid progenitor cells (MPC), lymphoid progenitor cells (LPC), lymphocytes, granulocytes, macrophages, erythrocytes, platelets, neutrophils, or natural kill cells. For example, the hematopoietic cells may be hematopoietic stem cells (HSC). In some instances, the hematopoietic cells may be one or more of a hematopoietic progenitor cell having the ability to differentiate into at least one of myeloid lineage, lymphoid lineage, megakaryocyte lineage, or erythroid lineage. In certain instances, the hematopoietic cells may be a plurality of different types of hematopoietic cells. In some cases, the hematopoietic cells may be partially or fully differentiated hematopoietic cells.

In some instances, the mesenchymal stromal cells may be autologous or allogeneic to the subject. In certain cases, the hematopoietic cells may be generated using mesenchymal stromal cells isolated from the bone marrow of the subject. Other sources of mesenchymal stromal cells include brain, adipose tissue, liver, lung, intestine, dental pulp, and umbilical cord tissue from the subject. In some examples, the hematopoietic cells may be generated using mesenchymal stromal cells isolated from a donor that is not the subject. The donor may be genetically related or unrelated to the subject. In one example, the mesenchymal stromal cell is isolated from an adult source. In another example, the mesenchymal stromal cell is isolated from adult bone marrow. The donor may be a living subject or a cadaver. Transplanting cells from a donor may be used in the context of many malignant and nonmalignant disorders to replace a defective subject marrow or immune system with a normal donor marrow and immune system. The degree of human leukocyte antigen (HLA) match between the donor and the subject is a factor in selecting a donor as well-matched transplants decrease the risk of graft rejection and graft versus host disease (GVHD), both of which are among the most serious sequelae of transplantation. The HLA genes fall in two categories (Type I and Type II). In general, mismatches of the Type-I genes (HLA-A, HLA-B, or HLA-C) increase the risk of graft rejection. A mismatch of an HLA Type II gene (HLA-DR or HLA-DQB1) increases the risk of GVHD.

In certain cases, mesenchymal stromal cells, or SIPs, may be propagated for at least 30 population doublings in vitro without senescing and without exhibiting any signs of genetic instability or loss of potential. In some instances, SIPs may be propagated in vitro for up to 30 population doublings. In one example, this can be performed using the methods described by Chamberlain et al. 2007. In some cases, adequate numbers of SIPs for reprogramming methods can be generated from a single marrow aspirate and used to produce substantial numbers of hematopoietic cells with the reprogramming efficiency described herein. In some cases, mesenchymal stromal cells may be isolated from the subject and cultured in vitro to produce sufficient quantities of hematopoietic cells for treating a subject having a diminished hematopoietic cell population. For example, mesenchymal stromal cells from a single bone marrow aspirate cultured in vitro may produce approximately 10⁹-10¹⁵ mesenchymal stromal cells (SIPs).

In some instances, the type of hematopoietic cell administered to the subject is selected based on the nature of the disease or disorder in which a subject's hematopoietic cell population is diminished. In some instances, the type of hematopoietic cell administered to the subject is selected based on what hematopoietic cell populations are diminished in the subject. In one example, reprogrammed HSC or erythrocytes may be administered to a subject having thalassemia or sickle cell anemia. In another example, where a subject has diminished platelet populations, reprogrammed megakaryocyte precursors, megakaryocytes, or platelets may be administered to the subject. In another example, reprogrammed HSC, lymphoid progenitors, or lymphocytes may be administered to a subject having severe combined immune-deficiency (SCID). In another example, where the subject has myelodysplastic syndrome (MDS), reprogrammed HSC may be administered to the subject. In another example, reprogrammed HSC may be administered to a subject having Fanconi anemia. In another example, where the subject has a hematopoietic-related malignancy, following radiotherapy and/or chemotherapy to remove the malignant cells, reprogrammed HSC may be administered to the subject. In some instances, the hematopoietic-related malignancy may be acute myeloid leukemia, chronic myeloid leukemia, acute lymphoblastic leukemia, Hodgkin lymphoma, non-Hodgkin lymphoma, or myelodysplastic syndrome (MDS). Reprogrammed HSC could also be used to repopulate or rescue the hematopoietic system of the subject following administration of radiotherapy and/or chemotherapy to treat a wide range of other cancers as well, including neuroblastoma, Ewing's sarcoma, gliomas, breast cancer, or other solid tumors.

The reprogrammed hematopoietic cells can be administered for therapeutic or prophylactic treatments or used in the laboratory. Thus, provided is a method of treating a blood disorder in a subject. The method includes administering the described reprogrammed hematopoietic cells to the subject. The cells may be administered intravenously or intravascularly. In certain instances, the cells are administered intravenously.

Optionally, the method may further include administering to the subject one or more additional therapeutic agents. For example, the therapeutic agent may be an immune suppressor. In other examples, the therapeutic agent may be a cancer therapy such as, for example, a chemotherapeutic agent. In other examples, a radiation therapy may be administered to the subject as a cancer therapy. In some instances, both radiation and a chemotherapeutic agent may be administered to a subject as a cancer therapy. In some instances, the therapeutic agent may be a hormone therapy. Such therapies are well known.

The combined administration contemplates co-administration of the hematopoietic cells and the therapeutic agent. In some instances, the reprogrammed cells and the therapeutic agent may a single pharmaceutical formulation. In other instances, the reprogrammed cells and the therapeutic agent may be separate pharmaceutical formulations. Combinations of agents or compositions can be administered separately but simultaneously (such as by separate intravenous lines) or sequentially (such as one agent or composition is administered first followed by administration of the second agent or composition). Thus, co-administration is used to refer to simultaneous or sequential administration of two or more agents or compositions as described herein. For example, in some instances, a therapeutic agent, or other treatment, such as a chemotherapeutic agent and/or radiation therapy, may be administered prior administration of the reprogrammed hematopoietic cells.

According to the methods provided herein, the subject is administered an effective amount the reprogrammed hematopoietic cells as described herein. The terms effective amount and effective number are used interchangeably. The term effective amount is defined as any amount necessary to produce a desired physiologic response (such as engraftment of hematopoietic cell populations in the subject). The number of hematopoietic cells administered, however, may be varied depending upon the requirements of the subject, the severity of the condition being treated, and the hematopoietic cell type being employed. For example, dosages can be empirically determined considering the type and stage of the disorder causing diminished hematopoietic cell populations diagnosed in a particular subject. The dose administered to a subject, in the context of the provided methods should be sufficient to affect a beneficial therapeutic response in the patient over time. Determination of the proper dosage for a particular situation is within the skill of the practitioner. Thus, effective amounts and schedules for administering the hematopoietic cells may be determined empirically by one skilled in the art. The ranges for administration are those large enough to produce the desired effect in which one or more symptoms of the disease or disorder are affected (such as reduced or delayed). The dosage should not be so large as to cause substantial adverse side effects, such as unwanted cross-reactions, anaphylactic reactions, and the like. Generally, the dosage may vary with the age, condition, sex, type of disease, the extent of the disease or disorder, route of administration, or whether other therapeutic agents are included in the regimen, and can be determined by one of skill in the art. The amount of cells administered can be adjusted by the individual physician in the event of any contraindications. The amount of cells administered can vary and can be administered in one or more dose administrations daily, for one or several days. Guidance can be found in the literature for appropriate treatment regimens involving administration of hematopoietic cells to a subject having diminished hematopoietic cell populations.

In some instances, the hematopoietic cells may be administered to the subject in sufficient number to repopulate the subject with hematopoietic cells. In some instances, the number of hematopoietic cells administered to the subject may be greater than or equal to about 5×10⁶ cells per kg (subject weight). In some cases, the hematopoietic cells are administered in a single administration. In other instances, the hematopoietic cells are administered in multiple administrations over time. In some instances, hematopoietic cells may be administered to the subject multiple times if the initial administration did not result in engraftment of the administered hematopoietic cells.

As used herein the terms treatment, treat, treating or ameliorating refers to a method of reducing the effects of a disease or condition or symptom of the disease or condition. Thus in the disclosed method, treatment can refer to a 5%, 10%, 20%, 30%, 40%, 50%, 60%, 70%, 80%, 90% or 100% reduction or amelioration in the severity of an established disease or condition or symptom of the disease or condition. For example, the method for treating a subject having a diminished hematopoietic cell population is considered to be a treatment if there is a 10% reduction in one or more symptoms of the disease in a subject as compared to control. Thus the reduction can be a 5, 10, 20, 30, 40, 50, 60, 70, 80, 90, 100% or any percent reduction in between 5 and 100 as compared to native or control levels. It is understood that treatment does not necessarily refer to a cure or complete ablation of the disease, condition or symptoms of the disease or condition. The treatment can be reflected, for example, in the presence of, or increase in population size, of hematopoietic cells in the subject. An exemplary treatment of a subject having a diminished hematopoietic cell population may be increasing the number of diminished hematopoietic cells in the system to within 5%, 10%, 20%, 30%, 40%, 50%, 60%, 70%, 80%, 90% or 100% of normal hematopoietic cell population size.

As used herein, the term subject can be a vertebrate, more specifically a mammal (for example, a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, whether male or female, are intended to be covered. As used herein, patient or subject may be used interchangeably and can refer to a subject with a disease or disorder. The term patient or subject includes human and veterinary subjects.

In some instances, provided is the use of the reprogrammed hematopoietic cells described herein, or generated using the methods described herein, for treatment of a subject having diminished hematopoietic cell populations. In other instances, provided is the use of hematopoietic cells generated from the mesenchymal stromal cells described herein for treatment of a subject having diminished hematopoietic cell populations. In these instances, the uses involve identifying a subject having diminished hematopoietic cells; and administering the hematopoietic cells to the subject to repopulate the subject with the hematopoietic cell population. In some instances, the hematopoietic cells are generated using mesenchymal stromal cells isolated from the bone marrow of the subject.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

Disclosed are materials, compositions, and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and discussed and a number of modifications that can be made to the combination, or methods of making or using such compositions, each and every combination and permutation of the composition or methods and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Those of ordinary skill in the art will realize that this description is illustrative only and is not intended to be in any way limiting. Other embodiments will readily suggest themselves to such skilled persons having the benefit of this disclosure. In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

All printed patents and publications referred to in this application are hereby incorporated herein in their entirety by this reference.

### EXAMPLES

While the invention will now be described in connection with certain specific examples in the following examples and with reference to the attached figures so that aspects thereof may be more fully understood and appreciated, it is not intended to limit the invention to these particular examples. On the contrary, this disclosure is intended to cover all alternatives, modifications and equivalents as may be included within the scope of the invention as defined by the appended claims. Thus, the following examples, which include specific embodiments, will serve to illustrate the practice and use of the methods and compositions described herein, it being understood that the particulars shown are by way of example and for purposes of illustrative discussion of specific examples only, and are presented in the cause of providing what is believed to be the most useful and readily understood description of formulation procedures, as well as of the principles and conceptual aspects of the invention.

### Example 1

### In Vivo Reprogramming of Stro1(+) Isolated Stromal Progenitors (SIPS) into Hematopoietic Cells

Summary: A human mesenchymal niche exists during human ontogeny that contributes to cartilage, bone, and vasculature. The fetal Stro-1+ cell population in this niche contains primitive APJ(APLNR)+/PDGFRα+ mesodermal progenitors and more differentiated VE-Cadherin+/VEGFR2+ hematovascular mesodermal precursors. Adult bone marrow (BM)-derived Stro-1+ cells are phenotypically and functionally different from fetal Stro-1+ cells. However, when placed into an in vivo fetal environment, adult BM-derived Stro-1+ cells reprogram to a more primitive phenotype, even at the clonal level, and generate hematopoietic stem cells (HSC) capable of robust serial, multi-lineage reconstitution. Adult Stro-1+ cells are a non-hematopoietic, epigenetically primed, somatic cell that is able to generate functional HSC in vivo.

A. Materials and Methods

Isolation, Characterization, and Culture of Bone Marrow-Derived Stro1+ Isolated Stromal Progenitors (SIPs).

Heparinized human bone marrow was obtained from healthy donors after informed consent according to guidelines from the Office of Human Research Protection at the University of Nevada, Reno, Wake Forest Health Sciences, or from commercially available sources (AllCells, LLC. Alameda Calif.). Low density BM mononuclear cells (BMNC) were separated by Ficoll density gradient (1.077 g/ml Sigma, St. Louis, Mo.) and washed twice in Iscove's modified Dulbecco's medium (Invitrogen, Carlsbad, Calif.). BMNC were first enriched for the Stro-1⁺ fraction using a Stro-1 antibody (R&D Systems, Minneapolis, Minn.) and magnetic bead cell sorting. (Miltenyi Biotec, Inc., Auburn, Calif.). In order to obtain clonally-derived cells, Stro-1⁺CD45⁻Gly-A⁻ cells were sorted into fibronectin-coated 96 well plates by single cell deposition using a FACSVantage (BD Biosciences, San Jose, Calif.). Twenty-four hours after single cell deposition, wells were visually inspected by phase contrast microscopy (Olympus IX70, Melville, N.Y.) to confirm the presence of a single cell/well. Only wells containing single cells were used to establish the clones employed in these studies. The number of wells positive for cellular growth that initially contained a single cell was 83%. Single cell populations were expanded in vitro at 37° C. in 5% CO₂ humidified air, in the presence of pre-screened mesenchymal stem cell growth media (MSCGM, Cambrex, Walkersville, Md.). At a cell confluence of 60-75%, clonal cells were detached with 0.25% trypsin (Invitrogen Corp., Carlsbad, Calif.) for 3-5 minutes at 37° C. Trypsin was neutralized with media containing fetal bovine serum (FBS), and cells were passaged at a 1:3 ratio in gelatin-coated tissue culture flasks.

Transplantation of SIPs into Fetal Sheep Recipients.

Fetal sheep (n=40) were injected intraperitoneally by ultrasound-guided transabdominal percutaneous injection (Chamberlain et al. 2007; Shaw et al. 2011) with SIP populations in QBSF serum-free media (Quality Biological, Gaithersburg, Md.) at the concentrations indicated in the Section B at 55-62 days of gestation and according to University of Nevada approved Institutional Animal Care and Use Committee (IACUC) guidelines. Briefly, after fetal visualization, a 22-gauge 15 cm echo-tip needle (Cook Medical Inc., Bloomington, Ind.) was inserted through the skin and the uterine wall into the amniotic cavity and then into the fetal peritoneal cavity under continuous ultrasound guidance. After confirmation of the appropriate positioning of the needle, the graft was injected slowly. The fetus was then checked to ensure adequate heartbeat after transplantation, just prior to anesthetic being withdrawn from the ewe.

Immunofluorescence of Bone Marrow Stromal Cells.

Stro-1+ human cells were isolated from bone marrow of secondary recipients using magnetic sorting as described above. Sorted cells were grown in chamber slides and fixed in 1% paraformaldehyde in PBS. Slides were washed, blocked in PBS containing 2% bovine serum albumin (BSA) (Sigma), and incubated in PBS containing 2% BSA and primary antibody overnight at 4° C. Primary antibodies were against Stro-1 (R&D Systems, Minneapolis, Minn.) and GFP (BioLegend, San Diego, Calif.). Slides were washed with PBS with 2% BSA and then incubated with secondary antibodies in PBS with 2% BSA for 1 hour at 4° C. Slides stained with secondary antibodies alone served as negative controls. Finally, cell nuclei were stained with DAPI (BioGenex, Fremont, Calif., USA) and coverslips mounted with Cytoseal 60 (Thermo Fisher Scientific Inc. Waltham, Mass., USA). An identical protocol was used for staining human fetal and adult Stro-1 positive cells, and in addition to the Stro-1 antibody, an anti-CD34 antibody (Abcam, Cambridge, Mass.) was also used.

Transplantation of CD34+CD45+ Cells Isolated from Bone Marrow of Primary Sheep SIPs Recipients into NOD/scid/γ(c)(−/−) (NSG) Mice.

Newborn NSG mice (1-3 day old) were irradiated with 100 cGy and injected intrahepatically as previously described (Strowig et al. 2011) with human CD34+CD45+ cells isolated from bone marrow of primary sheep SIPs recipients at the following doses: 2.5×10⁴, 1×10⁵, and 2.4×10⁵. In addition, and as a positive control, newborn NSG mice received CD34+CD117+ human mobilized peripheral blood stem cells (5.6×10⁴ (n=2) or 3.9×10⁵ (n=2)), while non-transplanted mice served as negative controls (n=2). Mice were maintained for 7-8 weeks in the Yale animal facility before being sacrificed for bone marrow and spleen analysis. Blood was collected at 5 and 7 weeks of age to test for human cell engraftment. All procedures were performed in compliance with relevant laws and institutional guidelines and were approved by the Yale University IACUC.

Flow Cytometry Analysis of Transplanted NSG Mice.

Peripheral blood was lysed twice using 1×BD Pharm Lyse™ solution (BD Biosciences, San Jose, Calif.) and resuspended in PBS+2% FBS. Cells were stained with antibodies against mouse CD45, and human MHC I, CD33, CD19, and CD3 (all from Biolegend San Diego, Calif.). For analysis of bone marrow (BM) and spleen, mice were euthanized at either 7 or 8 weeks of age. BM was recovered by flushing 1 femur and 1 tibia. Spleens were passed through 100 μM mesh in order to obtain a cell suspension of splenocytes. Cells were stained for flow cytometric analysis as described for peripheral blood above, additionally staining with antibodies against human CD2, CD7, CD11b, CD14, CD15, CD45RA, and CD34 (from BD and Biolegend San Diego, Calif.); anti-sheep CD45 (AbD Serotec); and anti-mouse CD45 and Ter-119. Cells were analyzed using a Stratedigm flow cytometer equipped with 4 lasers/13 detectors. Data were analyzed using FlowJo™ software. Viable cells were gated based on FSC and SSC parameters. Doublets were excluded by comparing FSC-H vs. FSC-A. When possible, unused fluorescence channels were used to exclude autofluorescent cells before gating for human and mouse CD45+ cells.

Angiogenic and Osteogenic Potential of Stro-1+ Cell Populations.

Tube formation assay was performed in 24 well plates by seeding each well with 5×10³ Stro-1+ cells (passage 4) that had been cultured and passaged in either MSCGM™ or EGM-2™ media (Lonza, Walkersville, Md.) onto 300 μl of polymerized Matrigel™ (BD Biosciences, Bedford, Mass.) at 37° C. in EGM-2™ medium supplemented with an extra 10% FBS (Lonza). Cells were then incubated at 37° C. and 5% CO₂ for 3-6 hours. The tube network formation was observed and quantified using a Zeiss inverted microscope. Osteogenic differentiation was performed as previously described (Chamberlain et al. 2007).

Preparation, Immunofluorescence and Flow Cytometric Analysis of Bone Tissue.

Human bone tissue was obtained from Advanced Bioscience Resources (Alameda, Calif.). Bone tissue older than 15 gestational weeks (gw) was decalcified using Decalcifying Solution-Lite (Sigma) until soft enough to be cut by a surgical blade. Bone tissue was then fixed in 10% neutral buffered formalin and paraffin embedded. Paraffin embedded 6 μm sections were prepared by de-waxing in xylene, rehydrating in decreasing concentrations of ethanol, and immersing in PBS. The primary antibodies used were: anti-Stro-1 (R&D Systems, Minneapolis, Minn.); anti-VEGFR2/FlK-1/KDR (Abcam, Cambridge, Mass.); anti-CD34 (Abcam, Cambridge, Mass.); anti-CD31 (Santa Cruz Biotechnology Dallas, Tex.); anti-VE-Cadherin (Abcam, Cambridge, Mass.); anti-N-Cadherin (BD Transduction Laboratories, San Jose, Calif.); anti-Osteopontin (Abcam, Cambridge, Mass.) and anti-CD45 (AbD Serotec, Raleigh, N.C.). Controls included slides stained in parallel, in which the primary antibody was either absent or was replaced by a non-specific isotype-matched primary antibody. An Olympus Fluoview™ 1000 confocal system was used to visualize and capture the fluorescent images. The number of total cells counted per tissue varied from 652-16,303 depending on the number of cells/slide. The numbers of positive cells in each section were determined as a percentage of the total number of cells. Flow cytometric analysis of fresh cells harvested from bone tissue was performed after bones were either minced or flushed with sterile media, and cells were collected and stained, as described above, with antibodies against human Stro-1, CD31, CD34 CD49f, CD90, CD117, CD140a, CD144, CD146, CD309 (all from BD Biosciences San Jose, Calif.). Cells were analyzed using a FACSCalibur® and data analyzed using FlowJo software.

Transduction of SIPs with Lentiviral Vectors Expressing EGPF.

Transduction of SIPs with the pEGFP-Lv105 vector (10¹⁰ pfu/ml) (Capital Biosciences, Rockville, Md.) was performed using subconfluent cultures of SIPs for 6 hours in QBSF60™ medium (Quality Biological) and 8 μg/mL protamine sulfate (Calbiochem, San Diego, Calif.) at a MOI of 100. After transduction, cells were washed and media was changed to MSCGM™. Efficiency of transduction (>95%) and viability of cells (>95%) was assessed prior to cell transplantation.

Assessment of Human Donor Hematopoietic Cell Engraftment.

Bone marrow and peripheral blood from animals transplanted with human cells were analyzed for the presence of human donor hematopoietic cells by flow cytometry using monoclonal antibodies (directly conjugated with FITC or PE) to CD3, CD7, CD10, CD13, CD20, CD34, CD45, CD33 (BD Biosciences, San Jose, Calif.), and glycophorin A (Beckman Coulter, Miami, Fla.) according to the manufacturers' recommendations. Flow cytometric analysis was performed using a FACScan™ (BD Biosciences) and the results were compared to those obtained when identical staining was performed with an aged-matched non-transplanted control animal.

Statistics and Data Analysis.

Experimental results are presented as the mean plus/minus the standard error of the mean (SEM). Comparisons between experimental results were determined by two-sided non-paired Student's t-test analysis. A p value <0.05 was considered statistically significant. The power of our statistical analysis was also validated using the two-sided Wilcoxon Rank Sum Test for independent samples.

B. Results

1. Analysis of Human Fetal Bone Shows that During Development Stro-1+ Cells Contribute to the Different Bone Marrow Niches

Flow cytometry and/or confocal microscopy were used to investigate the role of Stro-1+ cells in the development of the mesenchymal, vascular, and osteogenic niches during human bone marrow ontogeny. Human fetal bones were analyzed starting at 10 gestational weeks (gw) and for a period of approximately 10 weeks. At 10-12 gw, Stro-1+ CD34^{dim/-} chondrocytes (as described by Wu et al. 2013) were detected within the cartilaginous areas (FIG. 1A*), while in the developing bone marrow area Stro-1+ cells could be identified alongside sinusoidal endothelial cells co-expressing CD34+(FIG. 1A**) (n=4). Flow cytometric analysis confirmed that the large majority of the CD34+ cells isolated from fetal bones at 11-12 gw (47±4%) were vascular cells that did not express CD45, of which 74±5.4% were CD106+(VCAM-1), 65±7.2% were CD102+ (ICAM-1), and 10±0.5% were CD31+ (PECAM-1) (FIG. 1B) (n=4).

To assess whether Stro-1+ cells were part of the vasculature, the expression of other endothelial cell markers was investigated. Stro-1+ cells were found to co-expressed CD31, a marker of endothelial cells, and VE-cadherin, the presence of which is required for the organization of a stable vascular system (FIG. 1C). The percentage of Stro-1+ cells peaked at 11-12 gw to reach 27.47±0.49% and then decreased progressively over time due to the relative increase in hematopoietic cellularity of the bone marrow to 7.81±0.6% at 20 gw (FIG. 1D). VE-Cadherin expression mirrored that of Stro-1, being highly expressed early in gestation and decreasing with fetal bone maturation (FIG. 1D) (n=4).

Stro-1+ cells also expressed osteopontin and N-cadherin in areas of bone formation (FIG. 1E). Therefore, prior to the onset of fully developed hematopoietic production, Stro-1+ cells are present in both developing bone as well as the emerging vasculature.

Cells co-expressing Stro-1 and CD34 were assessed for expression of VEGFR2 (CD309) (n=3). VEGRF2/KDR/CD309 is essential for the development of both the hematopoietic and endothelial system and is a marker of hemogenic endothelium (Choi et al. 2012; Sturgeon et al. 2014). Starting at 10-12 gw in the vascular area of the bone, Stro-1+,CD34+,VEGFR2+ cells could be readily identified in forming vascular structures (FIG. 2A**). Later on in gestation, at 14 gw (FIG. 2B) and 18 gw (FIG. 2C), cells co-expressing Stro-1+,CD34+, and VEGFR2+ were also identified in specific locations within the vascular structures.

Flow cytometric evaluation of freshly isolated Stro-1+ cells (n=3) confirmed the results obtained by confocal microscopy, and showed that 23.9-42.3% of Stro-1+, CD34+ cells co-expressed CD31, and that Stro-1+, CD31+ cells that do not express CD34 were also present (4-19.7%). VEGFR2/KDR/CD309 was expressed in 7.6-39% of CD34+, Stro-1+ cells. In addition, 7.8-19.0% of Stro-1+, CD34+ cells were also positive for CD146 and 30.8-38-3% for CD49f. Also, most of the Stro-1+ cells expressed stem cell markers CD90 and CD117 (FIG. 2D).

Stro-1+ cells were assessed for expression of APLNR/APJ (n=3) (FIG. 2B). APLNR/APJ is a marker of early mesoderm (Vodyanik et al. 2010). Within the Stro-1+ cell population, 68.7-85.7% were positive for APJ, of which 15-19% were also CD140a/PDGFRα positive. This suggests that the Stro-1+ population within the fetus contains the most primitive mesodermal progenitors, according to the previously described hierarchy (Choi et al. 2012). In addition, 15.4-76% of Stro-1+ cells (depending on the samples analyzed) were also found to be positive for VE-Cadherin/CD144 and VEGFR2/KDR/CD309 (FIG. 2E). This suggests that more differentiated hematovascular mesodermal precursors are also present within the Stro-1 population.

Stro-1+ cells were also assessed for expression of CD45, which is characteristic of a hematopoietic phenotype. Starting at 14 weeks, it was possible to visualize the occasional presence of CD45+ hematopoietic cells that co-expressed Stro-1 (FIG. 3A).

2. Comparative Analysis of Adult and Fetal Stro-1+ Cells Demonstrate that Adult Stro-1+Cells Lose Expression of Endothelial Markers and are Unable to Efficiently Generate Capillary Tubes In Vitro

Flow cytometric analysis was used to assess endothelial markers in adult and fetal Stro-1+ cells. Cells were either assessed immediately after harvesting (fresh) or after culturing in vitro. Freshly isolated adult human bone marrow-derived Stro-1+ cells expressed less CD177 (1-5.1%), CD31 (0.83-1.2%), and less than 0.5% expressed CD34+(n=3) as compared to fetal Stro-1+ cells (FIG. 3B). Immunofluorescence analysis was also used to assess CD34 expression on fetal and adult Stro-1+ cells after culturing (fourth passage). Clusters of fetal Stro-1+ cells were found to co-express CD34, just as was seen within the in vivo microenvironment and in freshly isolated cells, while cultured adult BM derived Stro-1+ cells did not express CD34 (FIG. 3C) (n=3). These results were confirmed by RT-PCR, which demonstrated the presence of CD34 mRNA transcripts in cultured fetal Stro-1+ cells but not cultured adult Stro-1+ cells (FIG. 3D) (n=3).

Based on the assessed marker expression, the fetal BM, CD34+, Stro1+ cells seemed to be vascular in nature. As such, fetal and adult Stro-1+ cells were tested for their angiogenic potential using the tube formation assay after culturing at the same passage (P4). Fetal cells were significantly more efficient in generating capillary tubes than their adult counterparts (FIG. 3E). Furthermore, fetal but not adult cells were able to form vasculature without prior induction in culture (FIG. 3E and FIG. 3F) (n=3).

Expression of mesenchymal and hematovascular markers was also assessed. Both fetal and adult Stro-1+ cells expressed similar mesenchymal markers (CD105, CD146, CD90, CD45) (FIG. 3G) and were able to differentiate into osteocytes upon induction in appropriate media (FIG. 3H). However, in contrast to fetal Stro-1+ cells, fresh adult BM Stro-1+ cells did not express markers associated with hematovascular precursors but did express APLNR/APJ (FIG. 3I) (n=3). This is consistent with the mesoderm origin.

Thus, after in vitro culture, fetal Stro-1+ cells possessed both mesenchymal and vascular differentiative potential, while the adult bone marrow-derived Stro-1+ cells maintained a mesenchymal phenotype but had lost the ability to form vascular tubes without prior induction.

3. Adult Stro1+ Isolated Stromal Progenitors (SIPs) Reprogram In Vivo to Cells of the Hematopoietic Lineage after Transplantation into Fetal Sheep

The next set of experiments assessed whether exposing adult Stro-1+ cells to a fetal bone marrow microenvironment could induce these cells to return to a fetal-like phenotype and give rise to hematopoietic cells.

Stro-1⁺CD45⁻Gly-A⁻ cells (SIPs) isolated from human adult BM were grown in vitro and analyzed for different stem cell markers by flow cytometry (using methods described in Chamberlain et al. 2007; Sanada et al. 2013). It was previously established that SIPs express CD90, CD44, CD146, CD105, CXCL12, and CD29 but are devoid of the hematopoietic cell markers CD45, CD34, and CD133. Ten sheep fetuses were transplanted intra-peritoneally with SIPs at the following concentrations: 3.25×10⁵/fetus (n=3), 1.3×10⁶/fetus (n=3) and 1×10⁷/fetus (n=4). Animals were euthanized at 2 months post-transplant, and peripheral blood (PB) and bone marrow (BM) was collected to assess for human hematopoietic cell engraftment as determined by the total percentage of human myeloid and lymphoid cells. Results are shown in FIG. 4A. In PB and BM, the percentage of human hematopoietic cells varied from 0.47 to 6.54% and from 0.68 to 10.72%, respectively. While all transplanted animals harbored donor-derived lymphocytes, human myeloid and lymphoid cells were concurrently detected in 9 out of 10 of the recipients. Engraftment of lymphoid and myeloid cells, with reconstitution of both granulocytic and erythroid compartments, was detected in 50% of the animals, and 90% of recipients had donor-derived lymphoid and erythroid cells. Human CD34+ cells were found in 60% of the transplanted animals at levels ranging from 0.02 to 1.65%, and their levels did not directly correlate with either overall or multilineage engraftment. The overall percentage of engraftment within the different hematopoietic lineages is detailed in the left bar of FIG. 4B.

4. Hematopoietic Stem Cells Generated by In Vivo Reprogramming are Serially Transplantable

The next experiments assessed whether serially transplantable HSC were being generated from SIPs during this in vivo reprogramming. Serial transplantation studies were performed in which BM from primary SIPs sheep recipients were transplanted into eight secondary sheep recipients.

Analysis of human hematopoietic engraftment in the PB and BM of secondary recipients at 2 months post-transplant, as determined by the percentage of human myeloid and lymphoid cells, is shown in FIG. 4C. Human hematopoietic cells were detected in the PB and BM of all of the transplanted animals. The range of human hematopoietic cells was 2.17-6.86% and 0.46-3.14% in PB and BM, respectively. All transplanted animals harbored donor-derived lymphocytes and erythroid cells, while 80% had donor-derived lymphocytes, granulocytes, and erythrocytes. Human CD34+ cells (0.09-1.07%) were present in 60% of the transplanted animals, and the overall percentage of engraftment within the different hematopoietic lineages is detailed in the right bar of FIG. 4B. Representative dot plots from flow cytometric analysis of peripheral blood and bone marrow of primary and secondary recipients and a non-transplanted control are shown in FIG. 6A (peripheral blood) and FIG. 6B (bone marrow).

In the sheep model, cell transplantation must be performed during early gestation to avoid rejection of human cells. Thus, the possibility existed that the ability of in vivo reprogrammed SIPs to serially repopulate the hematopoietic system was unique to the fetal sheep milieu. To address this possibility, frozen bone marrow from primary SIPs recipients and isolated human CD34+CD45+ cells were used to perform a secondary transplant into NSG mice. Putative HSC generated by reprogramming of SIPs within the fetal sheep (labeled as “Donor (Ch)imeric (Sh)eep” in FIG. 4D) were capable of engrafting human CD45+ cells in 2 of 3 NSG mice for at least 8 wks.

5. SIPs that Did not Reprogram to the Hematopoietic Lineage are Also Serially Transplantable

Some studies in patients who received whole bone marrow transplantation suggested that stromal cells were not transplantable and could not engraft following infusion (Simmons et al. 1987), while other studies in patients with osteogenesis imperfecta suggested that stromal cells could engraft following transplantation (Otsuru et al. 2012). As such, experiments were performed to assess whether SIPs that did not reprogram to the hematopoietic lineage had the ability to engraft long-term as stromal cells and could serially engraft following transplantation into secondary recipients. Whole bone marrow (BM) was collected from two of the primary recipients that had received SIPs transduced with a lentiviral vector encoding Green Fluorescent Protein (GFP), and transplanted into two additional secondary recipients at a concentration of 3.7×10⁶ cells/fetus. The bone marrow samples were expected to contain both mesenchymal stromal cells and differentiated cell populations. Stro-1+ cells were then sorted from the BM of these two secondary recipients at 2.5 years post-transplant and grown in culture. Human Stro-1+, GFP+ cells were detected in culture (FIG. 4E). The human origin of these cells was confirmed by PCR analysis using human-specific primers for GAPDH (data not shown). This is the first study to show that stromal cells have the ability to be serially transplanted (namely, be transplanted and engraft in recipients).

6. Evaluation of Clonally-Derived SIPs for In Vivo Reprogramming to Cells of the Hematopoietic Lineage after Transplantation into Fetal Sheep

Experiments were performed to definitively show that the observed donor-derived hematopoiesis did not arise from undetectable levels of already committed hematopoietic cells. Clonally-derived SIPs (obtained as described in Section B) were expanded until sufficient cells were obtained from each clone for the experiments described below. Phenotypic characterization of the various resultant clones demonstrated these cells to be similar to non-clonally derived SIPs (data not shown). Each clonally-derived SIP population was transplanted into one fetal sheep at a concentration of 10⁶ cells/fetus (n=8). Recipients were evaluated at 75 days post-transplantation for the presence of in vivo-induced human hematopoietic cells. All eight transplanted SIP clones generated hematopoietic cells (FIG. 5A) at levels ranging from 1.25-7.63% and 8-13.73% in the BM and PB, respectively, including both myeloid and lymphoid cells, as determined by flow cytometry (FIG. 5B, left column). All transplanted animals harbored donor-derived myeloid and lymphoid cells. In 88% of the animals, donor-derived contribution to the granulocytic and erythroid compartments was also detected, and CD34⁺ cells were detected in all transplanted animals (0.22±0.05%).

Transplantation into secondary fetal sheep recipients (n=12) confirmed that the HSC generated from clonally-derived SIPs in the primary fetal sheep recipients were able to serially engraft. Flow cytometry was used to assess the total percentage of human cells in the PB (0.57-35%) and BM (0.45-25%) (FIG. 5C) CD34+ cells were present in 60% of these secondary recipients (0.53±0.14). The animals with the highest levels of engraftment also exhibited expansion of either the erythroid (Gly-A) or lymphoid (CD7) lineage (FIG. 5B, right column).

### Example 2

### In Vitro Reprogramming of Stro1(+) Isolated Stromal Progenitors (SIPs) into Hematopoietic Cells Using Vector-Expressed Transcription Factors

Summary:

Over-expressing OCT4 and/or the hematopoietic transcription factor (TF) RUNX1C in SIPs generates reprogrammed hematopoietic colonies by day 9 of culturing. SIPs-derived colonies induced by the OCT4+RUNX1C combination began expressing CD41, the earliest marker of definitive hematopoiesis, within 3-4 days. By day 5-6, ˜20% of SIPs were CD41+, expressed CD34 and CD45, and exhibited maximal induction of multiple hematopoiesis-specific transcription factors and phenotypic markers at 1-3 logs higher levels than human dermal fibroblasts (HFF). Replacing OCT4 with chromatin-remodeling agents further enhanced CD34 and CD41 expression, such that 35% and 10% of SIPs reprogrammed to express CD41 and CD34, respectively, within a 4 to 5 day period.

A. Materials and Methods

Isolation and Culture of Human Bone Marrow Mesenchymal Stromal Cells (BM-MSC).

Heparinized human BM was obtained from healthy donors after informed consent, according to guidelines from the Office of Human Research Protection at the University of Nevada at Reno, Wake Forest Health Sciences, or from commercially available sources (AllCells, LLC, Alameda Calif.). Low density BM mononuclear cells (BMNC) were separated by Ficoll density gradient (1.077 g/ml Sigma, St. Louis, Mo.) and washed twice in Iscove's modified Dulbecco's medium (Invitrogen, Carlsbad, Calif.). Stro-1+ MSC were then isolated using anti-Stro-1 antibody (R&D Systems, Minneapolis, Minn.) and magnetic bead cell sorting (Miltenyi Biotec, Inc., Auburn, Calif.) according to manufacturer's guidelines. Stro-1+ cells were then cultured at low density in 0.02% gelatin (Sigma, St Louis, Mo.) coated flasks using Mesenchymal Stem Cell Growth Medium (MSCGM™, Lonza, Walkersville, Md.) in a humidified 37° C. incubator at 5% CO₂.

Plasmid Construction and Lentiviral Vector Generation.

The plasmids pSin-EF2-OCT4-Pur, pSin-EF2RUNX1C-Pur, and pSin-EF2-eGFP-Pur, encoding the full-length cDNA of human OCT4, RUNX1C, and eGFP, respectively, were purchased from Addgene or constructed by subcloning the full-length cDNA for each gene into the Spe I and Eco RI sites in pSin-EF2-Nanog-Pur (No. 16578, Addgene, Cambridge, Mass.). As it was not commercially available in plasmid form, the full-length human RUNX1C cDNA (GenBank GI: 169790829) was custom synthesized by GenScript (Piscataway, N.J.) and subsequently subcloned into pSin-EF2-Nanog-Pur, replacing Nanog. The pSin-EF2-Oct4-Pur lentiviral vector encoding OCT4 was purchased from Addgene (No. 16579) and the eGFP sequence used was PCR cloned from the plasmid pCAG-GFP (Addgene Plasmid No. 11150). Alternative OCT4 cDNA sequences that could be used include, for example, GenBank GI Nos. 553727229, 553727232, 553727231, 553727228, or 553727227. There are also a large number of alternative eGFP sequences that could have been used. To produce lentiviral particles, 293T cells were plated at 5×10⁶ cells per 100 mm dish and incubated overnight. On the following day, the cells were transfected using FuGENE HD (Roche, Indianapolis, Ind.) with a mixture of the packaging constructs psPAX2 and pMD2.D (Addgene) and one of the vector backbone plasmids (pSin-EF2-OCT4-Pur, pSin-EF2-RUNX1C-Pur, or pSin-EF2-eGFP-Pur). Viral supernatant was collected at 48 and 72 hours after transfection, filtered through a 0.45 μm syringe filter (Millipore), and concentrated by Amicon Ultra Centrifugal Filter (Millipore).

BM-MSC Transduction and Induction of Cellular Reprogramming.

BM-MSC were transduced on 6-well plates with Oct4- or RUNX1C-encoding lentiviral vector, alone or in combination (or an eGFP-encoding lentiviral vector as a control), at a multiplicity of infection (MOI) of 50 for each vector. Twenty four hours after transduction, the vector-containing media was removed and replaced with fresh MSCGM™ (Lonza). At 48 hours post-transduction, BM-MSC were switched to a previously described induction media (Szabo et al. 2010), consisting of a 1:1 mixture of Dulbecco's modified Eagle's medium (DMEM)/Ham's F-12 (HyClone, South Logan, Utah), 10% Knockout Serum Replacement (KOSR; Invitrogen, Grand Island, N.Y.), supplemented with 30 μg/ml IGF-II (R&D Systems (Minneapolis, Minn.) and 20 μg/ml bFGF (STEMCELL Technologies, Vancouver, BC, Canada). In an effort to eliminate the need for Oct4, in some experiments, 1-5 μM Bix-01294 (Stemgent, San Diego, Calif.), an inhibitor of G9a histone methyltransferase, or 0.5-2.5 mM valproic acid (VPA; Sigma-Aldrich, St. Louis, Mo.), a histone deacetylase inhibitor, was included in the induction media to facilitate reprogramming.

RNA Isolation, cDNA Synthesis and Quantitative Real-Time PCR.

Total RNA was purified with an RNeasy Mini Kit (Qiagen, Valencia, Calif.) per the manufacturer's instructions. RNA concentrations were determined by a NanoDrop™ 2000 Spectrophotometer (Thermo Scientific, Wilmington, Del.). Total RNA (1 μg) was used to synthesize cDNA using random hexamer primers (Applied Biosystems, Foster City, Calif.). Equivalent amounts of cDNA were used for real-time PCR in a 20 μl reaction mixture with 10 μl of 2× SybrGreen™ PCR Mastermix (Life Technologies) and 1 μl of each specific primer pair. Reactions were run in triplicate with 40 cycles of amplification on an ABI Prism 7000 Real-Time PCR machine (Applied Biosystems, Foster City, Calif.). Primers for human CD34 (Catalog #PPH02455F) and CD45 (Catalog # PPH01510C) were purchased from SABiosciences (Qiagen). Primers used to amplify HOXB4 were previously described in Jackson et al. 2012. The sequences of the other primers used are listed in Table 1. The expression level of each mRNA was assessed after normalization of each sample relative to the expression of β-Actin measured in parallel reactions.

TABLE 1
Primers utilized for quantitative
RT-PCR amplification.

| Gene | Direction | Primer sequences |
|---|---|---|
| β-Actin | Forward | 5′-TCTGGCGGCACCACCATGTA-3′ |
| | Reverse | 5′-TTGCTGATCCACATCTGCTGG-3′ |
| GATA2 | Forward | 5′-GGGCTAGGGAACAGATCGACG-3′ |
| | Reverse | 5′-GCAGCAGTCAGGTGCGGAGG-3′ |
| KDR | Forward | 5′-GGCCCAATAATCAGAGTGGCA-3′ |
| | Reverse | 5′-CCAGTGTCATTTCCGATCACTTT-3′ |
| MIXL | Forward | 5′-GGATCCAGGTATGGTTCCAG-3′ |
| | Reverse | 5′-GGAGCACAGTGGTTGAGGAT-3′ |
| PU.1 | Forward | 5′-ATGACGTGTGTTGAACAAGACA-3′ |
| | Reverse | 5′-CGATGGTTGATTAAAGCCAGGT-3′ |
| RUNX1- | Forward | 5′-ACTCGGCTGAGCTGAGAAATG-3′ |
| Total | Reverse | 5′-GACTTGCGGTGGGTTTGTG-3′ |
| RUNX1C- | Forward | 5′-ATGGCTTCAGACAGCATATTTGA-3′ |
| Total | Reverse | 5′-GTGGACGTCTCTAGAAGGATT-3′ |
| RUNX1C- | Forward | 5′-GCCTTCAGAAGAGGGTGCAT-3′ |
| Endo | Reverse | 5′-GCACTGTGGGTACGAAGGAA-3′ |
| RUNX1C- | Forward | 5′-CCATTTCAGGTGTCGTGAGGA-3′ |
| Exo | Reverse | 5′-TGGCATCGTGGACGTCTCTA-3′ |
| WNT3 | Forward | 5′-AGGGCACCTCCACCATTTG-3′ |
| | Reverse | 5′-GACACTAACACGCCGAAGTCA-3′ |
| CDX4 | Forward | 5′-CCGATGCCAGCCTCCAATTT-3′ |
| | Reverse | 5′-CTGTGCCCATTGTACTAGACG-3′ |

Flow Cytometry.

BM-MSC, treated as indicated, were dissociated with trypsin/EDTA, stained with anti-CD34-PE, anti-CD45-PerCP (Becton-Dickinson Biosciences, San Jose, Calif., and anti-CD41-FITC (ABD Serotec, Raleigh, N.C.) on ice for 30 min, washed, and fixed with 4% paraformaldehyde (PFA). Analysis was performed using a FACSCalibur (Becton-Dickinson Biosciences).

Transcriptome Analysis of SIPs and HFF.

SIPs from 4 different human donors, isolated and grown as described above, were washed and lysed directly in Trizol (Life Technologies) and RNA was purified according to the manufacturer's instructions. The concentration of the RNA was determined by Ribogreen (Life Technologies), and RNA integrity was confirmed on an Agilent 2100 BioAnalyzer (Agilent Technologies, Santa Clara, Calif., USA). Total RNA samples (n=4) were labeled using an Affymetrix 3′ one-cycle cDNA Synthesis kit (Affymetrix, Santa Clara, Calif., USA) in accordance with manufacturer's protocols. Briefly, cDNA was synthesized from total RNA, followed by second-strand DNA synthesis. Biotin-labeled cRNA was synthesized, fragmented, and then hybridized to Affymetrix GeneChip Human Genome U133 Plus 2.0 arrays. The arrays were then washed and stained with streptavidin-phycoerythrin (Life Technologies) using the Affymetrix GeneChip Fluidics Station 450 and scanned using the Affymetrix GeneChip 3000 7G scanner. Probe-level analyses of the images from scanning of chips were performed using Affymetrix GeneChip Operating Software (GCOS). Threshold detection p-values were set to assign “present” (p<0.05), “marginal” (0.05≦p≦0.49), or “absent” (p>0.49) decision calls for each gene assigned by MAS 5.0 criteria using GCOS.

Immunofluorescence Staining.

Cells were fixed with 2% paraformaldehyde (PFA), permeabilized with 0.2% Triton-X100/PBS for 10 min at room temperature, blocked by blocking solution (Dako, Carpinteria, Calif.), and were then incubated with anti-RUNX1C antibody (OriGene), followed by incubation with Texas Red-conjugated secondary antibody (Vector Laboratories, Burlingame, Calif.). Nuclei were counterstained with 4,6-diamidino-2-phenylindole (DAPI) (Sigma-Aldrich).

Statistical Analysis.

Statistical analysis was performed using analysis of variance followed by Student's t-test. Data were processed using GraphPad Prism software (GraphPad Software, Inc., La Jolla, Calif.). For all analyses, a p value<0.05 was considered to be statistically significant.

B. Results

1. OCT4 Induces SIPs to Reprogram to the Hematopoietic Lineage Faster and More Efficiently than Fibroblasts

SIPs and human dermal (foreskin) fibroblasts (HFF) were transduced with OCT4 following the induction protocol described by Szabo et al. 2010. A direct head-to-head comparison was of morphology and gene/cell surface marker expression was performed using microscopy, microarray, qRT-PCR, and flow cytometry from day 3-16 post-transduction. Visual inspection of the cultures showed that reprogrammed colonies began to appear in SIPs cultures at day 9-10 (an image of a representative colony appears in FIG. 7), while rare colonies only began to appear in the HFF cultures at day 22-27, demonstrating that reprogramming proceeds 2-3 times faster and with greater efficiency in SIPs than in HFF.

Once reprogrammed colonies had begun forming in the cultures of Oct4-treated SIPs, the resultant colonies were live-stained with fluorescently-labeled CD45, CD34, SSEA4, and Tra-1-60 antibodies. Approximately 50% of the visible colonies (FIG. 8A) stained with CD45 (FIG. 8B) and CD34 (FIG. 8C), confirming successful reprogramming to early hematopoietic cells. When these images were merged (FIG. 8D), it can be seen that all stained cells were dual positive, i.e., expressed both CD34 and CD45, confirming their early hematopoietic identity. At no point during culture was staining ever seen with SSEA4 or Tra-1-60 (data not shown), confirming that this direct reprogramming process does not involve/require passage through an iPS-like state.

2. Transcriptional Profile of SIPs Primes them for Reprogramming to Generate HSC

Microarray analysis was performed on SIPs and HFF prior to and following transduction with an OCT4-encoding lentiviral vector as detailed in the Materials and Methods section. As can be seen in the heatmap in FIG. 9, clear differences were apparent between these cell types, both prior to and following forced expression of OCT4.

SIPs from four different human donors were analyzed with Affymetrix GeneChip Human Genome U133 Plus 2.0 arrays as detailed in the Materials and methods section. Particular attention was paid to gene signatures associated with the hemangioblast as well as genes linked with specification, emergence, and maturation of HSC during development. Amongst those associated with the hemangioblast, ANGPT1, KIT, MEIS1 and 2, NPR3, HEX, DLX5 were found to be expressed in SIPs, while CRHBP, GATA2, HLF, and PROM-1 were absent. With respect to those involved in hematopoietic pathway, SCL, RUNX1B, HHEX, KLF2, NFE2 were expressed in SIPs, while RUNX1C, LYL-1, LMO-2, GATA-2, PU.1, C-MYC ERG, FLI-1, GFIB, MLL, HOXB4, and C_{DX}4 were absent. In addition, SIPS had low/undetectable levels of MBD2 and MBD3, components of the nucleosome remodeling and deacetylation (NuRD) complex. A summary of these results is set forth below in Table 2. This analysis, combined with the data obtained from the preceding study of HFF and SIPs prior to and following forced expression of OCT4, provided a list of hemangioblast and hematopoiesis-associated genes that may be involved in the reprogramming of SIPs to HSC.

TABLE 2
Gene Expression Analysis.

| Hemangioblast-Associated Genes | | | Hematopoiesis-Associated Genes | | |
|---|---|---|---|---|---|
| Expressed: | Yes | No | Expressed: | Yes | No |
| Angpt1 | X | | Scl | X | |
| Kit | X | | Runx1b | X | |
| Meis1 | X | | Hhex | X | |
| Meis2 | X | | Klf2 | X | |
| Meis3 | X | | Nfe2 | X | |
| NPR3 | X | | Runx1c | | X |
| Hex | X | | Lyl-1 | | X |
| DLX5 | X | | Lmo-2 | | X |
| Crhbp | | X | Gata-2 | | X |
| Gata-2 | | X | Pu.1 | | X |
| Hlf | | X | c-Myc | | X |
| Prom-1 | | X | Erg | | X |
| | | | Fli-1 | | X |
| | | | Gfib | | X |
| | | | Mll | | X |
| | | | HoxB4 | | X |
| | | | Cdx4 | | X |

A number of transcription factors (TFs) play critical roles in regulating the fate of hematopoietic stem cells (Shivdasani and Orkin 1996). RUNX1C is a TF identified in the microarray analyses that is not basally expressed in SIPs but was induced by >3.5-fold upon forced expression of OCT4. RUNX1C is one of three transcripts produced from the runt-related transcription factor 1 (RUNX1) gene (Miyoshi et al. 1995). RUNX1 essential for the establishment of definitive hematopoiesis during embryogenesis (Chen et al. 2009, Ichikawa et al. 2008, Kurokawa 2006, Okuda et al. 1996, Tanaka et al. 2012), and the RUNX1C isoform is only expressed at the time of emergence of definitive HSCs, suggesting it may play a role in the establishment of hematopoiesis (Challen and Goodell 2011).

SIPs and HFF were transduced with (1) OCT4 alone or (2) OCT4+RUNX1C. A timeline of gene/cell surface marker expression was performed using microarray, qRT-PCR, and flow cytometry from day 3-16 post-transduction. Visual inspection of the cultures showed that reprogrammed colonies began to appear in SIPs cultures at day 9-10 following forced expression of either OCT4 alone or OCT4+RUNX1C. No colonies were seen during this time period in any of the HFF cultures. Flow cytometry (FIG. 10) on reprogramming SIPs demonstrated that expression of CD41, the earliest marker of commitment to the hematopoietic lineage (see Ferkowicz et al 2003, Mikkola et al. 2003), commenced in SIPs expressing OCT4+RUNX1C within only 3-4 days and peaked at day 5-6, by which time ˜20% of SIPs expressed this marker (n=5). As shown in FIG. 11, this observed peak in CD41 expression also coincided with commencement of mRNA expression of the phenotypic markers CD34 (induced by an average of 65-fold; range: 23.4- to 146-fold, n=6); CD45 (induced by an average of 295-fold; range: 25- to 430-fold, n=6), and KDR (induced by an average of 179-fold; range 21- to 369-fold, n=6), and maximal induction of mRNA expression of several hematopoiesis-specific transcription factors, including Pu.1, HOXB4, GATA2, MIXL, WNT3, C_{DX}4, and BMP4 (FIG. 12). Thus, the combination of OCT4+RUNX1C proved to be the most effective at inducing reprogramming of SIPs to the hematopoietic lineage. Interestingly, combining RUNX1C with OCT4 also enhanced the efficiency with which HFF were reprogrammed to the hematopoietic lineage. However, the levels of hematopoietic TFs and phenotypic markers which occurred following forced expression of OCT4+RUNX1C were 1-3 logs higher levels in SIPs than HFF, confirming the superiority of SIPs as a starting cell population for direct reprogramming in vitro to the hematopoietic lineage.

3. Direct Reprogramming of SIPs to the Hematopoietic Lineage without OCT4

In the above-described transduction experiments, expression of vector-encoded OCT4 and vector-driven RUNX1C continued unabated throughout the duration of culturing. However, when used as one of the Yamanaka factors to generate iPS cells (Takahasi et al. 2007, Takahashi and Yamanaka 2006), exogenous OCT4 expression (recombinant expression) is normally silenced when the cell has reached a state of induced pluripotency. As OCT4 expression is usually limited to highly primitive cells (Babaie et al. 2007, Campbell and Rudnicki 2013, Medvedev et al. 2008, Shin et al. 2013), it was possible that its continued expression could limit the efficiency and extent of SIP reprogramming to the hematopoietic lineage.

In an effort to further improve the efficiency of direct reprogramming, lentiviral-driven RUNX1C expression was combined with exposing the SIPs to two small molecules that modulate the activity of chromatin-modifying enzymes: Bix-01294, an inhibitor of the G9a histone methyltransferase; and valproic acid (VPA), a histone deacetylase inhibitor. As shown in FIG. 13, which displays the results of one representative experiment (n=4), it was possible to replace OCT4 with Bix-01294 and VPA and still achieve direct hematopoietic reprogramming of SIPs. Moreover, the elimination of OCT4 further enhanced the reprogramming process, such that 35% and 10% of SIPs reprogrammed to express CD41 and CD34, respectively, within a 4 to 5 day period. As can also be seen in FIG. 13, expressing RUNX1C alone in the absence of these small molecules was far less efficient at inducing reprogramming, suggesting that the chromatin must be opened and accessible for RUNX1C to bind its targets and drive direct hematopoietic reprogramming of SIPs. Exposing SIPs to induction media or these two small molecules alone did not induce significant expression of CD41 or CD34, confirming the essential role played by RUNX1C in this process. Flow cytometric analysis also demonstrated that emergent hematopoietic cells arising from the reprogrammed SIPs expressed the CD49f protein on their surface at levels of 8.4±1.2% (n=4). ITGA6, which encodes the integrin α6 (CD49f), has been reported to commence in hematopoietic precursor cells during ontogeny, and its expression has been shown to identify hematopoietic cells with long-term repopulating ability (Notta et al. 2011).

4. SIPs Continue to Express Exogenous RUNX1C after Direct Reprogramming

The length of time that RUNX1C continued to be expressed following transduction was assessed. The lentiviral vector used to express RUNX1C used the ubiquitously active EF1α promoter to drive RUNX1C transcription. However, during development, expression of RUNX1C is transient and is only required to trigger the onset of definitive hematopoiesis. Thus, its continued expression could limit the efficiency with which the generated CD41+ hematopoietic precursor cells are able to progress to CD34+ HSC. An upstream primer that recognized the 5′ untranslated region of RUNX1C (RUNX1C-Endo Forward) was used to distinguish endogenous gene expression from that of the lentiviral vector-derived RUNX1C expression (RUNX1C-Exo Forward). FIG. 14 shows the mRNA levels (assessed by qRT-PCR) of endogenous and exogenous RUNX1C, as well as total RUNX1. Forced over-expression of exogenous RUNX1C by the lentiviral vector inhibited the expression of the endogenous RUNX1C gene. In addition, expression of exogenous RUNX1C persisted at high levels, even at 12 days post-transduction, accounting for more than 90-95% of the total RUNX1C mRNA, and 74%-82% of the total RUNX1 within the cells. Recent studies have shown that hematopoietic differentiation of reprogrammed mouse embryonic fibroblasts only occurred once expression of the exogenously supplied hemogenic transcription factors was silenced (Pereira et al. 2013). Thus, the use of a lentivector with a tightly regulated regulatable promoter may further enhance the efficiency and completeness of hematopoietic reprogramming of SIPs.

### Example 3

### In Vitro Reprogramming of Stro1(+) Isolated Stromal Progenitors (SIPs) into Hematopoietic Cells Using Inducible-Expressed RUNX1C

An inducible promoter may be used to more tightly control RUNX1c expression. The lentivector employed in the experiments described in Example 2 drove continuous, high level expression of RUNX1c by virtue of its constitutive EF-1a promoter. As RUNX1c is only expressed transiently during development to initiate hematopoiesis, the continuous expression of RUNX1c may have reduced the generation of CD41+ pre-hematopoietic stem cells (pre-HSC). The use of an inducible promoter will permit expression of RUNX1c for an optimal duration and may result in increased reprogramming of SIPs to CD41+ pre-HSC.

As such, promoter systems with more tightly regulated gene expression will be assessed. Such promoter systems may include, for example, promoters such as the cumate switch (U.S. Pat. No. 7,745,592), the mifepristone-inducible mammalian expression system (for example, U.S. Pat. No. 7,241,744), hormone-modulated systems, small molecule-modulated systems (such as the rapamycin system), or a metal-inducible metallothionein promoter. For example, utilized promoters can be TDH3, ADH1, TPI1, ACT1, GPD or PGI or the galactose inducible promoters, GAL1, GAL7 and GAL10. The criteria for selecting the inducible promoter system is that it results in tight regulation of gene expression such that at most only a low level of the induced gene is expressed when the system is not induced. For example, initial studies using a Tetracycline (Tet)-inducible lentivector were performed. However, this inducible promoter system was too leaky to provide sufficiently tight regulation of gene expression (data not shown). Thus, the inducible promoter system selected may be selected to exhibit tighter gene expression control than the Tet-inducible system.

For example, RUNX1c may be cloned into a SparQ™ lentivector (System Biosciences). This vector employs a cumate “switch” (Mullick et al. 2006; U.S. Pat. No. 7,745,592) that provides extremely tight control, robust and dynamic inducibility, and highly titratable expression that is reversible and inducible repeatedly, with no loss in stringency. The resultant construct may be packaged into lentiviral particles using standard 2^{nd} or 3^{rd} generation protocols and concentrated 50-100× using Amicon Ultra 100K filters which are expected to produce a titer of approximately 10⁷ tu/ml with a variety of viral backbones.

Cultures may be performed in defined serum-free media. This will to facilitate clinical grade production of hematopoietic cells. SIPS (˜105) may be plated in 100 mm gelatin-coated culture plates using serum-free TheraPEAK® MSCGM-CD Mesenchymal Stem Cell Medium (Lonza). Sixteen hours later they may be transduced overnight with the lentivector encoding RUNX1c at an MOI of ˜10. A control group may be transduced with an identical lentivector expressing EGFP to judge transduction efficiency (usually ˜80-90%) and to assess the precision of the cumate “switch”. The following day, the medium may be changed to DMEM/F12/+20% KOSR/1×NEAA/0.1 mM 2-ME/containing rhFGF2 (20 ng/ml) and rhIGFII (30 ng/ml). Preliminary studies indicate that these two factors alone have some ability to support some degree of reprogramming of SIPs to the hematopoietic lineage (not shown). At the same time, media may be supplemented with cumate to turn on expression of RUNX1c (or EGFP) and with VPA (1 mM final concentration) and Bix-01294 (2 μM final concentration).

As described above, the combination of RUNX1c+VPA+Bix 01294 induces peak expression of multiple hematopoietic-specific transcription factors and surface markers within 4-5 days. Therefore, expression of RUNX1c may not be needed after that point. The optimal duration of RUNX1c expression may be determined using transduced cells divided into 5 experimental groups cultured for different durations in the presence of cumate: 1 day (d), 2d, 3d, 4d, and 5d. At each time points, cells may be extensively washed and fresh media added to the dish to turn off expression of RUNX1c or EGFP. At days 3, 4, 5, 6, and 7, representative cultures from each experimental group may be analyzed in situ for key markers of HSC. Wells may be briefly fixed in 4% paraformaldehyde, stained with fluorescent antibodies to CD41, CD34, CD45, CD49f, CD150, and CD133, washed, and examined using a multi-photon confocal microscope (Olympus FV1000MPE) to define the expression of these markers. Using a multi-photon confocal will enable characterization of the expression within the actual 3D clusters/colonies to assess the homogeneity of reprogramming.

To characterize the cellular composition of the cultures, at the same time points, parallel cultures may be dissociated with FACSmax® Dissociation Buffer (Amsbio Inc.) and stained with multiple fluorescent antibodies that detect markers in the hematopoietic continuum. CD41 may be assessed as a marker of early hematopoietic commitment. CD34, CD133, CD117 may be assessed as HSC markers. In addition, the following lineage (Lin) markers may be assessed: CD13, CD14, CD15, CD33, CD43, and CD123 may be assessed as myeloid lineage markers, and CD3, 5, CD7, CD10, and CD19 may be assessed as lymphoid lineage markers. CD45 will be used as a panhematopoietic marker. Cells that are CD45+CD34+Lin(−) may be used to represent the operational definition of a putative in vitro induced HSC

The duration of RUNX1c expression that yields the maximal number of CD45+CD34+Lin(−) putative HSC with minimal differentiation into other hematopoietic lineages will be identified.

### Example 4

### Culture Conditions to Maximize Reprogramming

Culture conditions will also be manipulated, including using the addition of cytokines, to improve two main parameters: (i) the number of CD34+45+Lin(−) colonies/cells generated from a given number of SIPs and (ii) the ability to separate and expand this population of reprogrammed cells. To generate more reprogrammed hematopoietic clusters/colonies, the SIP cultures will be maintained for longer periods, such as, for example, up to 14 days. In other conditions, the culture medium will also be supplemented with Flt-3 ligand (FL) and stem cell factor (SCF), which are cytokines that have been shown to increase by 4-6 fold the number of CD45+ cells obtained from reprogrammed fibroblasts (Szabo et al. 2010).

Once mature CD34+CD45+Lin(−) clusters/colonies have formed, they will be manually picked and expanded under serum-free conditions employing StemSpan 3000 (Stem Cell Technologies) supplemented with Flt-3 ligand (FL), SCF, IL-3 and IL-6. These factors are selected because SCF has been shown to improve homing capacity of HSC, FL leads to short term expansion and may help regulate the expression of the homing/adhesion receptor molecules VLA4/5, and IL-3/6 are essential cytokines for supporting or expanding early hematopoietic progenitors. After 7 days of culture, the increase in cell numbers will be assessed. Cells will also be evaluated by flow cytometry to assess the CD45+CD34+ phenotype and various markers of lineage commitment. The presence and level of VLA4 and CXCR4 homing receptors will also be measured, which can be predictive of efficient in vivo engraftment. Parallel cultures will be disaggregated into single cells using FACSmax buffer and CD34+CD45+Lin(−) cells, isolated from the total culture by sorting on the FACS Aria, will be expanded under the same conditions. This will account for circumstances where not all cells within a colony will be of the correct phenotype and any CD34+CD45+ cells that might not reside within the clusters/colonies proper.

In order to facilitate differentiation of the HSC into all lineages or into hematopoietic cells having good engraftment potential, additional growth factors, such as TPO and BMP4, may be incorporated into the culture medium. Addition of VEGF and SCF and the removal of VPA and Bix-01294 at very specific times during the 5-day reprogramming process increases the percentage of cells expressing HSC markers such as CD34 and c-kit, while decreasing the percentage of cells expressing very early hematopoietic markers like CD49f (data not shown). In addition, culture media may also be supplemented with signals other than hematopoietic growth factors, such as, for example, Notch, Wnt, BMP-4 and Tie2/angiopoietin-1) and intracellular mediators (phosphatase and tensin homolog and glycogen synthase kinase-3) to promote generation and expansion of HSC without differentiation (Hofmeister et al. 2007). The transcriptome of HSC generated via reprogramming of SIPs will be compared to the transcriptome of HSC, isolated directly from bone marrow to select specific pathways for modulation to further improve reprogramming efficiency and fidelity.

### Example 5

### Methods of Treatment

Reprogrammed hematopoietic cells as described herein would be administered to a subject for a transplant in the same manner that naturally-occurring (isolated) hematopoietic cells are currently used for therapy. A brief description is provided below of a general protocol for a hematopoietic stem cell (HSC) transplant for a subject having cancer in which the subject's own own hematopoietic cells were damaged or depleted by radiotherapy or chemotherapy. However, modifications to this protocol are generally known, as are similar methods for the administration of other types of hematopoietic cells to subjects as therapy for other disorders, such as, for example, disorders of red blood cells such as thalassemia or sickle cell anemia, disorders in platelets, disorders in immune cell function such as severe combined immune-deficiency (SCID), as well as disorders like, myelodysplastic syndrome (MDS), aplastic anemia, and Fanconi anemia, amongst others.

Following appropriate radiotherapy or chemotherapy to ablate the endogenous hematopoietic system (the precise preconditioning regimen would be determined by the disease to be treated), reprogrammed HSCs may be infused through a central vein over a period of several hours. The transplantation uses about 5×10⁶ cells per kg (subject weight). If the cells have been cryopreserved, to avoid the risk of encephalopathy, which occurs with doses above 2 g/kg/day of DMSO (common additive to cryopreserved cells), stem cell infusions exceeding 500 mL are infused over 2 days and the rate of infusion is limited to 20 mL/min. The HSC subsequently engraft within the bone marrow cavity of the subject. After several weeks of growth in the bone marrow, expansion of HSC and their progeny is sufficient to normalize the blood cell counts and reinitiate the subject's immune system.

### REFERENCES

In the foregoing discussion, certain articles and processes are described for background or introductory purposes or to explain certain aspects of the disclosure. Nothing contained herein is to be construed as an “admission” of prior art.
Almeida-Porada G., et al. (2000). Generation of hematopoietic and hepatic cells by human bone marrow stromal cells in vivo. Blood 96, 570a (Abstract).
Almeida-Porada G, et al. (2000). Cotransplantation of human stromal cell progenitors into preimmune fetal sheep results in early appearance of human donor cells in circulation and boosts cell levels in bone marrow at later time points after transplantation. Blood. 95(11):3620-7.
Babaie, Y., et al. (2007). Analysis of Oct4-dependent transcriptional networks regulating self-renewal and pluripotency in human embryonic stem cells. Stem Cells 25, 500-10.
Campbell, P. A., and Rudnicki, M. A. (2013). Oct4 interaction with Hmgb2 regulates Akt signaling and pluripotency. Stem Cells 31, 1107-20.
Challen, G. A., and Goodell, M. A. (2011). Runx1 isoforms show differential expression patterns during hematopoietic development but have similar functional effects in adult hematopoietic stem cells. Exp Hematol 38(5), 403-16.
Chamberlain, J., et al. (2007). Efficient generation of human hepatocytes by the intrahepatic delivery of clonal human mesenchymal stem cells in fetal sheep. Hepatology 46(6), 1935-45. Chen, M. J., et al. (2009). Runx1 is required for the endothelial to haematopoietic cell transition but not thereafter. Nature 457, 887-91.
Choi, K. D., et al. (2012). Identification of the hemogenic endothelial progenitor and its direct precursor in human pluripotent stem cell differentiation cultures. Cell Rep 2, 553-567.
Delort, J. P., and Capecchi, M. R. (1996). TAXI/UAS: a molecular switch to control expression of genes in vivo. Hum. Gene Ther. 7, 809-820.
Ferkowicz, M. J., et al. (2003). CD41 expression defines the onset of primitive and definitive hematopoiesis in the murine embryo. Development 130(18), 4393-403.
Gossen, M., and Bujard, H. (1992). Tight control of gene expression in mammalian cells by tetracycline-responsive promoters. Proc. Natl. Acad. Sci. U.S.A. 89, 5547-5551.
Gossen, M., et al. (1995). Transcriptional activation by tetracyclines in mammalian cells. Science 268, 1766-1769.
Hanna, J., et al. (2007). Treatment of sickle cell anemia mouse model with iPS cells generated from autologous skin. Science 318(5858), 1920-3.
Hexum, M. K., et al. (2011). In vivo evaluation of putative hematopoietic stem cells derived from human pluripotent stem cells. Methods Mol Biol 767, 433-47.
Hofmeister C C, et al. (2007). Ex vivo expansion of umbilical cord blood stem cells for transplantation: growing knowledge from the hematopoietic niche. Bone Marrow Transplant. 39(1):11-23.
Ichikawa, M., et al. (2008). AML1/Runx1 negatively regulates quiescent hematopoietic stem cells in adult hematopoiesis. J Immunol 180, 4402-8.
Jackson, M. et al. (2012). HOXB4 can enhance the differentiation of embryonic stem cells by modulating the hematopoietic niche. Stem Cells. (2):150-60.
Kurokawa, M. (2006). AML1/Runx1 as a versatile regulator of hematopoiesis: regulation of its function and a role in adult hematopoiesis. Int J Hematol 84, 136-42.
Makarov, S., et al. (1994). Hyperinducible human metallothionein promoter with a low level basal activity. Nucl. Acids Res. 2(8):1504-1505.
McKinney-Freeman, S. L., et al. (2009). Surface antigen phenotypes of hematopoietic stem cells from embryos and murine embryonic stem cells. Blood 114(2), 268-78.
Medvedev, S. P., et al. (2008). [OCT4 and NANOG are the key genes in the system of pluripotency maintenance in mammalian cells]. Genetika 44, 1589-608.
Mikkola, H. K., et al. (2003). Expression of CD41 marks the initiation of definitive hematopoiesis in the mouse embryo. Blood 101(2), 508-16.
Miyoshi, H., et al. (1995). Alternative splicing and genomic structure of the AML1 gene involved in acute myeloid leukemia. Nucleic Acids Res 23, 2762-9.
Mullick A, et al. The cumate gene-switch: a system for regulated expression in mammalian cells. BMC Biotechnol. 2006; 6:43.
Ning, H., et al. (2011). Mesenchymal stem cell marker Stro-1 is a 75 kd endothelial antigen. Biochemical and biophysical research communications 413, 353-357.
No, D., et al. (1996). Ecdysone-inducible gene expression in mammalian cells and transgenic mice. Proc. Natl. Acad. Sci. U.S.A. 93, 3346-3351.
Notta, F., et al. (2011). Isolation of single human hematopoietic stem cells capable of long-term multilineage engraftment. Science 333, 218-21.
Okuda, T., et al. (1996). AML1, the target of multiple chromosomal translocations in human leukemia, is essential for normal fetal liver hematopoiesis. Cell 84, 321-30.
Otsuru S. et al. (2012). Transplanted bone marrow mononuclear cells and MSCs impart clinical benefit to children with osteogenesis imperfecta through different mechanisms. Blood. 120(9):1933-41.
Pereira, C. F., et al. (2013). Induction of a hemogenic program in mouse fibroblasts. Cell Stem Cell 13, 205-18.
Rivera, V. M., et al. (1996). A humanized system for pharmacologic control of gene expression. Nat. Med. 2, 1028-1032.
Robin, C., et al. (2011). CD41 is developmentally regulated and differentially expressed on mouse hematopoietic stem cells. Blood 117, 5088-91.
Sanada C, et al. (2013). Mesenchymal stem cells contribute to endogenous FVIII:c production. J. Cellular Physiology. 228(5):1010-16.
Sakamoto, H., et al. (2010). Hematopoiesis from pluripotent stem cell lines. Int J Hematol 91, 384-91.
Shaw, S. W., et al. (2011). Autologous transplantation of amniotic fluid-derived mesenchymal stem cells into sheep fetuses. Cell Transplant 20, 1015-1031.
Shin, D. M., et al. (2013). Very small embryonic-like stem-cell optimization of isolation protocols: an update of molecular signatures and a review of current in vivo applications. Exp Mol Med 45, e56.
Shivdasani, R. A., and Orkin, S. H. (1996). The transcriptional control of hematopoiesis. Blood 87, 4025-39.
Shoshani, O., et al. (2014). Cell isolation induces fate changes of bone marrow mesenchymal cells leading to loss or alternatively to acquisition of new differentiation potentials. Stem Cells. 32(8):2008-20.
Simmons et al. (1987). Host origin of marrow stromal cells following allogeneic bone marrow transplantation. Nature. 328(6129):429-32.
Soland, M. A., et al. (2014). Perivascular stromal cells as a potential reservoir of human cytomegalovirus. Am J Transplant 14, 820-830.
Sturgeon, C. M., et al. (2014). Wnt signaling controls the specification of definitive and primitive hematopoiesis from human pluripotent stem cells. Nature biotechnology 32, 554-561.
Strowig, T., et al. (2011). Transgenic expression of human signal regulatory protein alpha in Rag2−/−gamma(c)−/− mice improves engraftment of human hematopoietic cells in humanized mice. Proc Natl Acad Sci USA 108, 13218-13223.
Szabo, E., et al. (2010). Direct conversion of human fibroblasts to multilineage blood progenitors. Nature 468(7323), 521-6.
Szulc, J., et al. (2006). A versatile tool for conditional gene expression and knockdown. Nat. Methods 3, 109-116.
Takahashi, K., et al. (2007). Induction of pluripotent stem cells from adult human fibroblasts by defined factors. Cell 131, 861-72.
Takahashi, K., and Yamanaka, S. (2006). Induction of pluripotent stem cells from mouse embryonic and adult fibroblast cultures by defined factors. Cell 126, 663-76.
Tanaka, Y., et al. (2012). The transcriptional programme controlled by Runx1 during early embryonic blood development. Dev Biol 366, 404-19.
Vodyanik, M. A., et al. (2010). A mesoderm-derived precursor for mesenchymal stem and endothelial cells. Cell Stem Cell 7, 718-729.
Wang, Y., et al. (1994). A regulatory system for use in gene transfer. Proc. Natl. Acad. Sci. U.S.A. 91, 8180-8184.
Woods, N. B., et al. (2011). Brief report: efficient generation of hematopoietic precursors and progenitors from human pluripotent stem cell lines. Stem Cells 29(7), 1158-64.
Wu, L., et al. (2013). Human developmental chondrogenesis as a basis for engineering chondrocytes from pluripotent stem cells. Stem Cell Reports 1, 575-589.
Ye, L., et al. (2013). Blood cell-derived induced pluripotent stem cells free of reprogramming factors generated by Sendai viral vectors. Stem Cells Transl Med 2(8), 558-66.
Zhou W and Freed CR. (2009). Adenoviral gene delivery can reprogram human fibroblasts to induced pluripotent stem cells. Stem Cells. 27(11):2667-74.
Zou, J., et al. (2011). Site-specific gene correction of a point mutation in human iPS cells derived from an adult patient with sickle cell disease. Blood 118(17), 4599-608.

1. A hematopoietic cell comprising a vector, the vector comprising a nucleic acid sequence encoding a transcription factor that controls hematopoietic cell differentiation.

2. The hematopoietic cell of claim 1, wherein the hematopoietic cell has been reprogrammed from a mesenchymal stromal cell that expresses Stro1 but does not express CD34, CD45, and GlyA.

3. The hematopoietic cell of claim 2, wherein the mesenchymal stromal cell was isolated from bone marrow.

4. The hematopoietic cell of claim 2, wherein the reprogramming of the mesenchymal stromal cell into a hematopoietic cell was caused by expression of the transcription factor.

5. The hematopoietic cell of claim 1, wherein the transcription factor is RUNX1C or OCT4.

6. The hematopoietic cell of claim 1, wherein the vector comprises a regulatable promoter operably linked to the nucleic acid sequence encoding the transcription factor.

7. The hematopoietic cell of claim 6, wherein the regulatable promoter permits gene expression to be turned on by addition of an inducer or turned off by addition of a repressor, wherein no or little expression occurs in the absence of the inducer or in the presence of the repressor.

8. (canceled)

9. (canceled)

10. The hematopoietic cell of claim 1, wherein the hematopoietic cell comprises a vector comprising a nucleic acid sequence encoding RUNX1C and a vector comprising a nucleic acid sequence encoding OCT4.

11. The hematopoietic cell of claim 10, wherein at least one of the vectors comprises a regulatable promoter operably linked to the nucleic acid sequence encoding RUNX1C or OCT4.

12. (canceled)

13. (canceled)

14. The hematopoietic cell of claim 1, wherein the hematopoietic cell is a hematopoietic stem cell (HSC), a hematopoietic progenitor cell (HPC), a myeloid progenitor cell (MPC), a lymphoid progenitor cell (LPC), a lymphocyte, a granulocyte, a macrophage, an erythrocyte, or a platelet.

15. (canceled)

16. (canceled)

17. (canceled)

18. A method of generating a hematopoietic cell according to claim 1, comprising:
(a) transducing an isolated mesenchymal stromal cell with the vector comprising a nucleic acid sequence encoding a transcription factor that controls hematopoietic cell differentiation;
(b) culturing the isolated mesenchymal stromal cell in culture medium after transducing the isolated mesenchymal stromal cell with the vector; and
(c) harvesting a cell population enriched for hematopoietic cells.

19. (canceled)

20. The method of claim 18, wherein the culture medium comprises a compound that alters chromatin modification.

21. The method of claim 20, wherein the compound that alters chromatin modification is a chromatic-modifying enzyme inhibitor.

22. (canceled)

23. (canceled)

24. (canceled)

25. A method of treating a subject having a diminished hematopoietic cell population, the method comprising:
(a) identifying a subject having diminished hematopoietic cells;
(b) providing hematopoietic cells according to claim 1; and
(c) administering the hematopoietic cells to the subject to repopulate the subject with the hematopoietic cell population.

26. The method of claim 25, wherein the hematopoietic cells were generated using mesenchymal stromal cells isolated from the bone marrow of the subject.

27. An isolated mesenchymal stromal cell comprising a vector, the vector having a nucleic acid sequence encoding a transcription factor that controls hematopoietic cell differentiation.

28. The mesenchymal stromal cell of claim 27, wherein the mesenchymal stromal cell expresses Stro1 but does not express CD34, CD45, and GlyA.

29. The mesenchymal stromal cell of claim 27, wherein the mesenchymal stromal cell was isolated from bone marrow.

30. The mesenchymal stromal cell of claim 27, wherein expression of the transcription factor triggers reprogramming of the mesenchymal stromal cell into hematopoietic cell.

31. The mesenchymal stromal cell of claim 27, wherein the transcription factor is RUNX1C or OCT4.

32. The mesenchymal stromal cell of claim 27, wherein the vector comprises a regulatable promoter operably linked to the nucleic acid sequence encoding the transcription factor.

33. The mesenchymal stromal cell of claim 32, wherein the regulatable promoter permits gene expression to be turned on by addition of an inducer or turned off by addition of a repressor, wherein no or little expression occurs in the absence of the inducer or in the presence of the repressor.

34. (canceled)

35. (canceled)

36. The mesenchymal stromal cell of claim 27, wherein the hematopoietic cell comprises a vector comprising a nucleic acid sequence encoding RUNX1C and a vector comprising a nucleic acid sequence encoding OCT4.

37. The mesenchymal stromal cell of claim 36, wherein at least one of the vectors comprises a regulatable promoter operably linked to the nucleic acid sequence encoding RUNX1C or OCT4.

38. (canceled)

39. (canceled)

40. The mesenchymal stromal cell of claim 27, wherein the mesenchymal stromal cell reprograms upon expression of the transcription factor into a hematopoietic stem cell (HSC), a hematopoietic progenitor cell (HPC), a myeloid progenitor cell (MPC), a lymphoid progenitor cell (LPC), a lymphocyte, a granulocyte, a macrophage, a erythrocyte, or a platelet.

41. (canceled)

42. (canceled)

43. (canceled)
